SEQUENCE LISTING (26 sequences)

SEQ ID NO: 1 - protein, 13 aa, Artificial Sequence
  Description of Artificial Sequence Synthetic peptide
CELCCNPACAGCY

SEQ ID NO: 2 - protein, 18 aa, Escherichia coli
  See specification as filed for detailed description of substitutions and preferred embodiments
NTFYCCELCCNPACAGCY

SEQ ID NO: 3 - protein, 14 aa, Artificial Sequence
  Description of Artificial Sequence Synthetic peptide
CCELCCNPACAGCY

SEQ ID NO: 4 - protein, 1073 aa, Homo sapiens
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQILMIAVFTLTGAVVLLLLVALLMLRKYRKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANISQKGDVYSYGIIAQEIILRKETFYTLSCRDRNEKIFRVENSNGMKPFRPDLFLETAEEKELEVYLLVKNCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICKYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 5 - protein, 5 aa, Artificial Sequence
  Description of Artificial Sequence Synthetic peptide
GYYWS

SEQ ID NO: 6 - protein, 16 aa, Artificial Sequence
  Description of Artificial Sequence Synthetic peptide
EINHRGNTNDNPSLKS

SEQ ID NO: 7 - protein, 11 aa, Artificial Sequence
  Description of Artificial Sequence Synthetic peptide
ERGYTYGNFDH

SEQ ID NO: 8 - protein, 11 aa, Artificial Sequence
  Description of Artificial Sequence Synthetic peptide
RASQSVSRNLA

SEQ ID NO: 9 - protein, 7 aa, Artificial Sequence
  Description of Artificial Sequence Synthetic peptide
GASTRAT

SEQ ID NO: 10 - protein, 9 aa, Artificial Sequence
  Description of Artificial Sequence Synthetic peptide
QQYKTWPRT

SEQ ID NO: 11 - protein, 119 aa, Artificial Sequence
  Description of Artificial Sequence Synthetic polypeptide
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 12 - protein, 107 aa, Artificial Sequence
  Description of Artificial Sequence Synthetic polypeptide
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 13 - protein, 468 aa, Artificial Sequence
  Description of Artificial Sequence Synthetic polypeptide
MGWSCIILFLVATATGVHSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 233 aa, Artificial Sequence
  Description of Artificial Sequence Synthetic polypeptide
MGWSCIILFLVATATGVHSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 5 aa, Artificial Sequence
  Description of Artificial Sequence Synthetic peptide
SHRMN

SEQ ID NO: 16 - protein, 16 aa, Artificial Sequence
  Description of Artificial Sequence Synthetic peptide
IITHNSITYYASWAKS

SEQ ID NO: 17 - protein, 10 aa, Artificial Sequence
  Description of Artificial Sequence Synthetic peptide
EDSMGYYFDL

SEQ ID NO: 18 - protein, 11 aa, Artificial Sequence
  Description of Artificial Sequence Synthetic peptide
QASQSISNWLA

SEQ ID NO: 19 - protein, 7 aa, Artificial Sequence
  Description of Artificial Sequence Synthetic peptide
RASTLAS

SEQ ID NO: 20 - protein, 12 aa, Artificial Sequence
  Description of Artificial Sequence Synthetic peptide
QQTYTNNHLDNG

SEQ ID NO: 21 - protein, 5 aa, Artificial Sequence
  Description of Artificial Sequence Synthetic peptide
NYAIS

SEQ ID NO: 22 - protein, 16 aa, Artificial Sequence
  Description of Artificial Sequence Synthetic peptide
YISYGKSIYYASWAKG

SEQ ID NO: 23 - protein, 10 aa, Artificial Sequence
  Description of Artificial Sequence Synthetic peptide
EDSATYSPNL

SEQ ID NO: 24 - protein, 11 aa, Artificial Sequence
  Description of Artificial Sequence Synthetic peptide
QASQSINTYLA

SEQ ID NO: 25 - protein, 7 aa, Artificial Sequence
  Description of Artificial Sequence Synthetic peptide
RASTLAS

SEQ ID NO: 26 - protein, 12 aa, Artificial Sequence
  Description of Artificial Sequence Synthetic peptide
QQGYSYNNLDRA

(19) United States
(12) Patent Application Publication
(10) Pub. No.: US 2016/0303258 A1
(43) Pub. Date: Oct. 20, 2016

(54) COMPOUNDS AND COMPOSITIONS FOR IMAGING GCC-EXPRESSING CELLS

(71) Applicants: MILLENNIUM PHARMACEUTICALS, INC., Cambridge, MA (US); INVICRO LLC, Boston, MA (US); UNIVERSITY OF IOWA RESEARCH FOUNDATION, Iowa City, IA (US); STC.UNM, Albuquerque, NM (US)

(72) Inventors: Donna Cvet, Arlington, MA (US); Swapnil Raut, Woburn, MA (US); Michael K. Schultz, Iowa City, IA (US); Jeffrey P. Norenberg, Albuquerque, NM (US); Tamara Anderson Daniels, Albuquerque, NM (US); John William Hoppin, Boston, MA (US)

(21) Appl. No.: 15/100,883

(22) PCT Filed: Dec. 3, 2014
(86) PCT No.: PCT/US14/68431

§ 371 Date: Jun. 1, 2016

Related U.S. Application Data
(60) Provisional application No. 61/911,323, filed on Dec. 3, 2013.

(51) Int. Cl. A61K 51/04; A61K 51/08; G01N 33/60; A61K 45/06; G01N 33/50; C07K 7/08; C07K 16/40

(52) U.S. Cl. CPC A61K 51/0482; C07K 7/08; A61K 51/08; C07K 16/40; A61K 45/06; G01N 33/5011; G01N 33/60

(57) ABSTRACT

This disclosure provides radiolabeled compounds that bind to guanylyl cyclase C (GCC) and which can bind cancer cells that express GCC. Exemplary compounds comprise a chelating moiety capable of binding a radioactive atom, a peptide capable of binding GCC, and a linker moiety connecting the two. This disclosure also provides methods of detecting and treating cancer using the compounds described herein.

### SEQUENCE LISTING

The instant application contains a Sequence Listing which has been submitted electronically in ASCII format and is hereby incorporated by reference in its entirety. Said ASCII copy, created on Nov. 14, 2014, is named M2051-7039WO_SL.txt and is 23,344 bytes in size.

### BACKGROUND

Colorectal cancer (CRC) is the third most common cancer in men and the second most common in women worldwide. The highest incidence rates are estimated in Australia/New Zealand and Western Europe, the lowest in Africa (except Southern Africa) and South-Central Asia, and are intermediate in Latin America. About 608,000 deaths from CRC are estimated annually worldwide, making it the fourth most common cause of death from cancer. The highest mortality rates are in Central and Eastern Europe, and the lowest are in Middle Africa. Prognosis of CRC is primarily related to stage, histologic and molecular differentiation, lymphovascular invasion, and extent of tumor-free surgical resection margins.

Guanylyl cyclase C (GCC) is a transmembrane cell surface receptor that functions in the maintenance of intestinal fluid and electrolyte homeostasis, and cell proliferation. GCC is expressed in an anatomically privileged location in the brush border membranes on the luminal side of intestinal epithelial cells in healthy individuals. This location limits targeting by intravenously (IV) administered agents. However, on malignant transformation, this privileged location is lost in primary and metastatic intestinal tumors but GCC expression is maintained. Immunohistochemistry (IHC) analysis of a large number of human metastatic cancer tumor tissues demonstrates that approximately 90% of colorectal, 75% of gastric, and 65% of pancreatic tumor tissues express GCC.

There are currently GCC-targeted cancer therapeutics in development, such as MLN0264, that specifically bind and kill cancer cells that express GCC. To predict the efficacy of a GCC-targeted therapeutic, it is desirable to first know whether a tumor expresses GCC. This determination is typically made by IHC. IHC is technically limited because it requires invasive collection of a biopsy sample, and only assays the biopsy sample rather than the whole tumor or tumors.

### SUMMARY

This disclosure provides radiolabeled compounds that bind to guanylyl cyclase C (GCC) and detect cancer cells that express GCC. Typically, in a healthy patient, GCC is only expressed in epithelial cells on the apical side of the intestine and in dopamine neurons in the brain. However, cancers of gastrointestinal origin express GCC and expression is maintained in tumor metastases. Certain non-gastrointestinal cancer cells also express GCC. In general, the compounds comprise a chelating moiety capable of binding a radioactive atom, a peptide capable of binding GCC, and a linker moiety connecting the two. The GCC-binding peptides are related to E. coli enterotoxin, an 18 amino acid sequence (SEQ ID NO: 2) that is a natural ligand for GCC. The chelator can be any moiety capable of stably binding a radioactive atom, for example a macrocycle comprising N or O atoms.

This disclosure also provides methods of labeling the peptide-linker-chelator compound with a radiolabel. In addition, the disclosure provides methods of using the compound for tumor imaging, including determining whether a patient can be treated with a GCC-targeted therapeutic. For instance, when the compound comprises a positron-emitting moiety, it can be detected by in vivo detection methods such as, e.g., PET. The disclosure also provides methods of using the compound to treat diseases associated with aberrant GCC expression, optionally in combination with other therapies, e.g., a GCC-targeted therapy.

This disclosure provides, e.g., compounds that are capable of binding to guanylyl cyclase C (GCC). The compounds may comprise, e.g., a peptide capable of binding to GCC (e.g., a peptide as described herein) and a chelating moiety (e.g., a chelating moiety as described herein). The compound may comprise a linker, e.g., a linker as described herein, that covalently attaches the amino acid to the chelating moiety.

In some aspects, the present disclosure provides a compound comprising: (a) a peptide comprising an amino acid sequence of SEQ ID NO: 1, or an amino acid sequence at least 75% identical to SEQ ID NO:1, wherein the amino acid sequence has an amino terminus and has a free carboxy-terminus, (b) a chelating moiety capable of binding a positron-emitting atom, and (c) a linker moiety that covalently attaches the amino-terminus of the amino acid sequence of the peptide to the chelating moiety, wherein the compound is capable of binding to guanylyl cyclase C (GCC). In some aspects, the present disclosure provides a compound comprising: (a) a peptide comprising an amino acid sequence of SEQ ID NO: 1, wherein the peptide has an amino terminus and has a free carboxy-terminus, (b) a chelating moiety capable of binding a radioactive atom, wherein the chelating moiety comprises a macrocycle, e.g., a macrocycle comprising an O and/or a N, DOTA, NOTA, one or more amines, one or more ethers, one or more carboxylic acids, EDTA, DTPA, TETA, DO3A, PCTA, or desferrioxamine, and (c) a linker moiety that covalently attaches the amino-terminus of the amino acid sequence of the peptide to the chelating moiety, wherein the compound is capable of binding to guanylyl cyclase C (GCC). In some aspects, the present disclosure provides a compound comprising: (a) a peptide comprising an amino acid sequence of SEQ ID NO: 1, wherein the peptide has an amino terminus and has a free carboxy-terminus, (b) a chelating moiety capable of binding a positron-emitting atom, (c) a linker moiety that covalently attaches the amino-terminus of the amino acid sequence of the peptide to the chelating moiety, and (d) a positron-emitting atom, e.g., gallium-68, wherein the compound is capable of binding to guanylyl cyclase C (GCC).

In embodiments, the peptide comprises a GCC-binding portion of the amino acid sequence of SEQ ID NO: 2. In embodiments, the peptide comprises the amino acid sequence of SEQ ID NO: 3. In embodiments, disulfide bonds connect Cys5 to Cys10, Cys6 to Cys14, and Cys9 to Csy17, wherein the amino acids are numbered according to their position in native E. coli enterotoxin. In embodiments, the peptide consists of the amino acid sequence of SEQ ID NO: 2. In embodiments, the peptide consists of the amino acid sequence of SEQ ID NO:1.

In some embodiments, the chelating moiety is capable of binding a radioactive atom. The binding may be direct, e.g., the chelating moiety may make hydrogen bonds or electrostatic interactions with the radioactive atom. The binding may also be indirect, e.g., the chelating moiety binds to a molecule that comprises a radioactive atom. In embodiments, the chelating moiety comprises, or is, a macrocycle. In embodiments, the chelating moiety comprises, or is, DOTA or NOTA. In embodiments, the chelating moiety comprises a macrocycle, e.g., a macrocycle comprising an O and/or a N, DOTA, NOTA, one or more amines, one or more ethers, one or more carboxylic acids, EDTA, DTPA, TETA, DO3A, PCTA, or desferrioxamine.

In embodiments, the linker moiety comprises, or is, an aminopentyl, aminohexyl, or aminoheptyl group. In embodiments, the linker moiety comprises an alkylene, e.g., C1-C10 alkylene such as C1, C2, C3, C4, C5, C6, C7, C8, C9, or C10 alkylene. In embodiments, the linker moiety comprises O or S. In embodiments, the linker moiety comprises urea or thiourea. In embodiments, the linker moiety comprises a benzyl group.

In embodiments, the compound is a 2,2′,2″,2′″-((S)-2-(4-(3-(6-(((3S,6R,9S,15R,20R,23S,26S,29R,32R,37R,40S,45aS)-40-(2-amino-2-oxoethyl)-15-(((S)-1-carboxy-2-(4-hydroxyphenyl)ethyl)carbamoyl)-26-(2-carboxyethyl)-23-isobutyl-3,9-dimethyl-1,4,7,10,13,22,25,28,31,38,41,47-dodecaoxotetracontahydro-1H-37,20-(epiminomethano)-6,29-(methanodithiomethano)pyrrolo[2,1-s][1,2,27,28,5,8,11,14,17,20,23,32,35,38,41]tetrathiaundecaazacyclotritetracontin-32-yl)amino)-6-oxohexyl)thioureido)benzyl)-1,4,7,10-tetraazacyclododecane-1,4,7,10-tetrayl)tetraacetic acid.

In embodiments, the compound has the structure of Formula (VI):

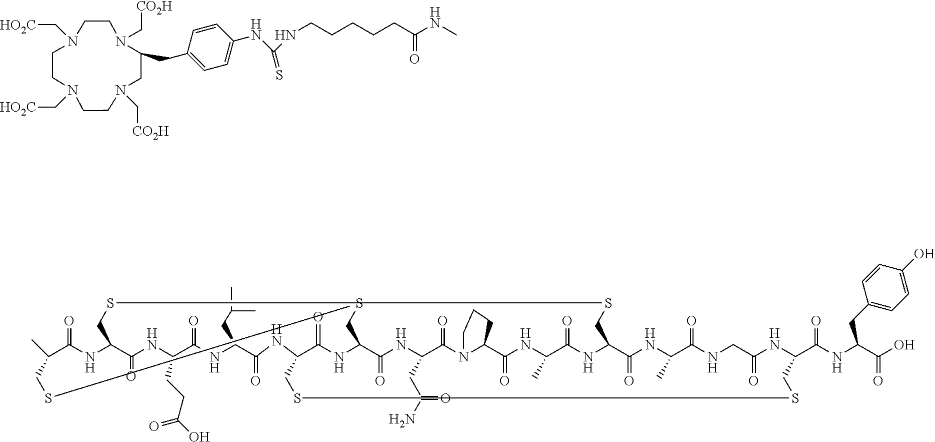 [SMILES: CNC(=O)CCCCCNC(=S)Nc1ccc(C[C@H]2CN(CC(=O)O)CCN(CC(=O)O)CCN(CC(=O)O)CCN2CC(=O)O)cc1 ; CC(C)C[C@@H]1NC(=O)[C@H](CCC(=O)O)NC(=O)[C@@H]2CSSC[C@@H]3NC(=O)[C@H](C)NC(=O)[C@@H]4CCCN4C(=O)[C@H](CC(N)=O)NC(=O)[C@H](CSSC[C@H](C)C(=O)N2)NC(=O)[C@H](CSSC[C@@H](C(=O)N[C@@H](Cc2ccc(O)cc2)C(=O)O)NC(=O)CNC(=O)[C@H](C)NC3=O)NC1=O]

In embodiments, the compound further comprises a radioactive atom bound to the chelating moiety. In embodiments, the radioactive atom is a positron-emitter. In embodiments, the radioactive atom is Gallium-68. In embodiments, the radioactive atom is an alpha particle emitter. In embodiments, the radioactive atom is 213Bi, 90Y, or 177Lu. In embodiments, the radioactive atom is ²¹³Bi or ²²⁵Ac. In embodiments, the radioactive atom is a beta-emitter, e.g., ⁹⁰Y or ¹⁷⁷Lu. In embodiments, the radioactive atom is a gamma-emitter. In embodiments, the radioactive atom is 111In. In embodiments, the compound has a specific activity of about 20 to 40 MBq/nmol, e.g., 25-33 MBq/nmol. In embodiments, the compound has a specific activity of about 29 MBq/nmol.

In embodiments, the compound has the structure of Formula (VIa):

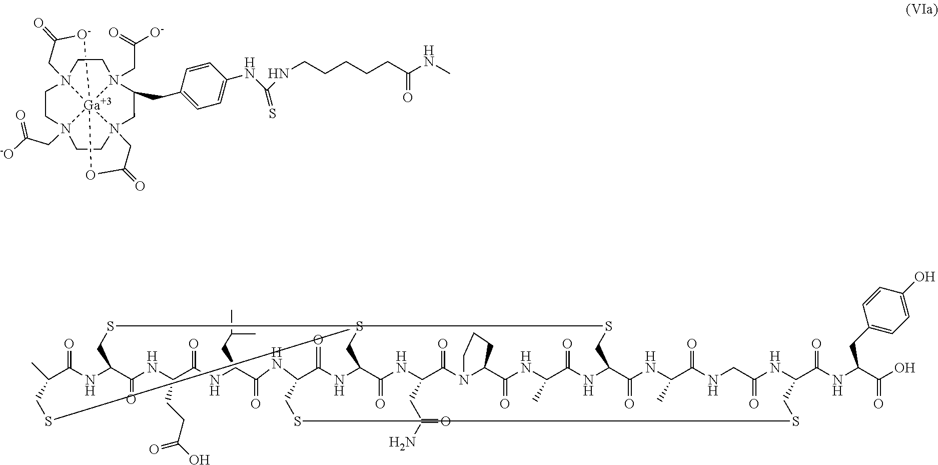

In certain aspects, this disclosure also provides a compound according to Formula II, IV or V. In embodiments, the peptide comprises the amino acid sequence of SEQ ID NO:1. In embodiments, the peptide comprises the amino acid sequence is SEQ ID NO: 3.

In embodiments, the chelating moiety comprises one or more amines, ethers or carboxylic acids. In embodiments, the chelating moiety comprises a plurality of amines. In embodiments, the chelating moiety is a macrocycle, e.g., a macrocycle comprising an O and/or a N. In embodiments, the chelating moiety comprises DOTA, NOTA, EDTA, DTPA, TETA, DO3A, PCTA, or desferrioxamine. In embodiments, the chelating moiety is DOTA.

In embodiments, the linker is an aminopentyl, aminohexyl, or aminoheptyl group. In embodiments, the linker is aminohexyl.

In embodiments, the compound further comprises a radioactive atom bound to the chelating moiety, e.g., the compound of Formula I, III, IVa or Va. In embodiments, the radioactive atom is a positron-emitter. In embodiments, the radioactive atom is Gallium-68. In embodiments, the radioactive atom is an alpha particle emitter. In embodiments, the radioactive atom is 213Bi, 90Y, or 177Lu. In embodiments, the compound has a specific activity of about 25-33 MBq/nmol. In embodiments, the compound has a specific activity of about 29.4 MBq/nmol.

The present disclosure also provides, in some aspects, a composition comprising a plurality of compounds described herein, wherein at least one compound of the plurality is bound to a radioactive atom, e.g., a positron-emitting atom, e.g., Gallium-68, and at least one compound of the plurality is not bound to a radioactive atom.

In embodiments, at least one of the compounds of the plurality has a structure of Formula (I), e.g., a structure of Formula (III), and at least one of the compounds of the plurality has a structure of Formula (II). In embodiments, at least one of the compounds of the plurality has a structure of Formula (Va), and at least one of the compounds of the plurality has a structure of Formula (V). In embodiments, at least one of the compounds of the plurality has a structure of Formula (VIa), and at least one of the compounds of the plurality has a structure of Formula (VI). In embodiments, the composition has a ratio of about 1:100 to 1:10,000 of Gallium-68-bound compounds to unbound compounds. In embodiments, the composition has a ratio of about 1:1,000 to 1:2,000 of Gallium-68-bound molecules to unbound molecules. In embodiments, the composition has a ratio of about 1:1,500 of Gallium-68-bound molecules to unbound molecules.

In certain aspects, the present disclosure comprises composition comprising: (a) a compound as described herein, e.g., an unlabeled GCC-binding compound comprising a chelating moiety that is capable of binding a radioactive atom, e.g., a compound of Formula (VI), and one or more of, e.g., all of, (b) an alcohol, e.g., ethanol, (c) a salt, e.g., a sodium salt, e.g., sodium acetate, and (d) water. In embodiments, the composition has a pH of about 3-6, e.g., about 3-4, 4-5, or 5-6, e.g., about 3.8.

In embodiments, the alcohol, e.g., ethanol is present at about 10-25%, e.g., about 10-15%, 15-20%, or 20-25%.

In embodiments, the composition further comprises one or more of, e.g., all of, a sugar (e.g., sucrose), acetic acid (e.g. glacial acetic acid), a polysorbate (e.g., polysorbate 80), and a surfactant (e.g., a non-ionic surfactant, e.g., Pluronic F-68).

In embodiments, the compound has a concentration of about 0.01-0.04 mg/ml, or at least about 0.01 mg/ml, or at most about 0.04 mg/ml. In embodiments, the compound has a concentration of about 0.01-0.04 μg/ml, or at least about 0.01 μg/ml, or at most about 0.04 μg/ml.

The present disclosure also provides, in certain aspects, a composition comprising a GCC-binding compound as described herein, in aqueous solution, wherein the compound has a concentration of about 0.01-0.04 μg/ml, or at least about 0.01 mg/ml, or at most about 0.04 mg/ml. In embodiments, the concentration is about 0.027 ug/ml.

The present disclosure also provides, in certain aspects, a composition comprising a GCC-binding compound as described herein, e.g., an unlabeled GCC-binding compound, in aqueous solution, wherein the compound has a concentration of at least about 0.027 ug/ml.

The present disclosure also provides, in certain aspects, a container comprising a composition as described herein, e.g., a composition comprising an unlabeled GCC-binding compound and one or more of ethanol, water, and sodium acetate, wherein optionally the pH is between about 3 and about 6. In embodiments, the composition has a volume of about 2 ml.

The present disclosure also provides, in certain aspects, a kit comprising a container as described herein, e.g., a container comprising an unlabeled GCC-binding compound, and a cassette suitable for contacting the compound with radioactive material.

The kit may further comprise written instructions for radiolabeling the compound with the radioactive material.

The present disclosure also provides, in certain aspects, a composition comprising: (a) a compound as described herein, e.g., a GCC-binding compound bound to a radioactive atom, and one or more of, e.g., all of, (b) an alcohol, e.g., ethanol, (c) a salt, e.g., a sodium salt, e.g., sodium chloride, and (d) water.

In some embodiments, the composition comprises: (a) a compound as described herein, e.g., a GCC-binding compound bound to a radioactive atom, (b) ethanol, (c) sodium chloride, and (d) water. In some embodiments, the compound is a compound of Formula (VI). In embodiments, the radioactive atom is Gallium-68.

In embodiments, the alcohol, e.g., ethanol is present at about 5-25%, e.g., about 10-15%, 15-20%, or 20-25%, or at about 8%. In embodiments, the salt, e.g., sodium chloride is present at about 0.3-1.4%, e.g., about 0.3%-0.5%, 0.5%-0.8%, 0.8%-0.9%, 0.9%-1.1%, or 1.1%-1.4%.

In embodiments, the composition has a volume of about 8-11 ml, e.g., about 8-9, 9-10, or 10-11 ml, or about 9.5 ml.

In embodiments, the composition comprises about 5.1-7.6 mCi of radioactivity, or about ≧12.73 mCi of radioactivity.

In embodiments, the composition comprises about 20-70 μg of the compound, e.g., about 20-30, 30-40, 40-50-, 50-60, or 60-70 μg, or about 55.0 μg of the compound.

The present disclosure also provides, in certain aspects, a composition comprising a compound as described herein, e.g., a radioactively labeled GCC-binding compound as described herein, e.g., the compound of Formula (VIa) and a pharmaceutically acceptable excipient.

The present disclosure also provides, in certain aspects, a container comprising a composition as described herein, e.g., a composition as described above. In embodiments, the container is a vial.

In certain aspects, the present disclosure provides a kit comprising a composition or container as described herein. In embodiments, the kit further comprises written instructions for a method of administering the compound to a subject.

In certain aspects, the present disclosure provides a method of assaying for a GCC-expressing cell in a subject, comprising: (a) administering, e.g., intravenously, a compound as described herein, e.g., a radioactively labeled GCC-binding compound, e.g., the compound of Formula (VIa) to the subject, and (b) visualizing the distribution of radioactivity in the subject.

In embodiments, the compound is a compound of Formula (VI) bound to Gallium-68. In embodiments, the radioactivity is visualized by positron emission tomography (PET). In embodiments, the compound administered has about 4.0 to 6.0 (±10%) mCi, e.g., about 4.0-5.0, about 5.0-6.0, or about 5.0 mCi. In embodiments, the compound is administered at a dose of about 20-60 μg, about 30-50 μg, about 35-45 μg, or about 40-46 μg. In embodiments, the compound is administered at a dose of about 43.4 μg.

In embodiments, the subject is a human.

In embodiments, the subject is suffering from a disorder associated with GCC expression, such as cancer. In embodiments, the disorder is selected from solid tumor, a soft tissue tumor, a metastatic lesion, a sarcoma, an adenocarcinoma, or a carcinoma. In embodiments, the cancer is early or late stage cancer, or cancer of any of stages 0, 1, IIA, IIB, IIIA, IIIB, IIIC, and IV. In embodiments, the disorder is selected from colorectal cancer, gastric cancer, esophageal cancer, pancreatic cancer, lung cancer, leiomyosarcoma, rhabdomyosarcoma, and a neuroendocrine tumor, or any metastases thereof. In embodiments, the colorectal cancer is a colorectal adenocarcinoma, colorectal leiomyosarcoma, colorectal lymphoma, colorectal melanoma, or colorectal neuroendocrine tumor. In embodiments, the gastric cancer is a gastric adenocarcinoma, gastric sarcoma, or gastric lymphoma. In embodiments, the esophageal cancer is esophageal squamous cell carcinoma, or esophageal adenocarcinoma. In embodiments, the lung cancer (e.g., non-small cell or small cell lung cancer) is a squamous cell carcinoma or adenocarcinoma. In embodiments, the neuroendocrine tumor is a gastrointestinal or a bronchopulmonary neuroendocrine tumor.

In embodiments, the method further comprises administering an agent that ameliorates bladder toxicity associated with the administered compound. The agent that ameliorates bladder toxicity may increase urinary excretion. The agent that ameliorates bladder toxicity may be, e.g., saline or water.

In embodiments, the method further comprises performing partial volume correction on an image representing the distribution of radioactivity in the patient.

The present disclosure also provides, in certain aspects, a method of determining sensitivity of cancer cells to a GCC-targeted therapeutic agent, comprising assaying for a GCC-expressing cell in the subject according to a method as described herein, wherein binding of the radiolabel to the cancer cell indicates sensitivity to the GCC-targeted therapeutic agent.

The present disclosure also provides, in certain aspects, a method of evaluating whether a subject is a potential candidate for a GCC-targeted therapy, comprising assaying for a GCC-expressing cell in the subject according to a method as describe herein, wherein binding of the radiolabel to the cancer cell indicates that the subject is a potential candidate for a GCC-targeted therapy

The present disclosure also provides, in certain aspects, a use of the compound as described herein, e.g., a radiolabeled GCC-binding compound, e.g., a compound of Formula (VIa) for detecting a GCC-expressing cell in a subject.

The present disclosure also provides, in certain aspects, a use of the compound as described herein, e.g., a radiolabeled GCC-binding compound, e.g., a compound of Formula (VIa) in the manufacture of a composition for detecting a GCC-expressing cell in a subject.

The present disclosure also provides, in certain aspects, a method of treating a subject having a disorder characterized by one or more GCC-expressing cells, comprising: (a) assaying for a GCC-expressing cell in the subject according to the method as described herein, and (b) if the subject has one or more cells that express GCC, administering a GCC-targeted therapeutic agent.

In embodiments, the GCC-targeted therapeutic agent comprises an anti-GCC antibody molecule selected from: (a) an anti-GCC antibody molecule comprising three heavy chain complementarity determining regions (CDR1, CDR2, and CDR3) comprising amino acid sequences provided in Table 3; and three light chain complementarity determining regions (CDR1, CDR2, and CDR3) comprising amino acid sequences provided in Table 3. (b) an anti-GCC antibody molecule that competes for binding or binds to the same epitope as an anti-GCC antibody comprising three heavy chain complementarity determining regions (CDR1, CDR2, and CDR3) comprising amino acid sequences provided in Table 3 and three light chain complementarity determining regions (CDR1, CDR2, and CDR3) comprising amino acid sequences provided in Table 3, and (c) an anti-GCC antibody molecule that is capable of competing for binding or capable of binding to the same epitope as an anti-GCC antibody comprising three heavy chain complementarity determining regions (CDR1, CDR2, and CDR3) comprising amino acid sequences provided in Table 3 and three light chain complementarity determining regions (CDR1, CDR2, and CDR3) comprising amino acid sequences provided in Table 3. In embodiments, the GCC-targeted therapeutic agent comprises an anti-GCC antibody molecule selected from: (a) an anti-GCC antibody molecule comprising a heavy chain CDR1 of SEQ ID NO: 5, a heavy chain CDR2 of SEQ ID NO: 6, a heavy chain CDR3 of SEQ ID NO: 7, a light chain CDR1 of SEQ ID NO: 8, a light chain CDR2 of SEQ ID NO: 9, and a light chain CDR3 of SEQ ID NO: 10, (b) an anti-GCC antibody molecule that is capable of competing for binding with an anti-GCC antibody comprising three heavy chain complementarity determining regions (CDR1, CDR2, and CDR3) comprising a heavy chain CDR1 of SEQ ID NO: 5, a heavy chain CDR2 of SEQ ID NO: 6, a heavy chain CDR3 of SEQ ID NO: 7, a light chain CDR1 of SEQ ID NO: 8, a light chain CDR2 of SEQ ID NO: 9, and a light chain CDR3 of SEQ ID NO: 10, and (c) an anti-GCC antibody molecule that is capable of binding to the same epitope as an anti-GCC antibody comprising a heavy chain CDR1 of SEQ ID NO: 5, a heavy chain CDR2 of SEQ ID NO: 6, a heavy chain CDR3 of SEQ ID NO: 7, a light chain CDR1 of SEQ ID NO: 8, a light chain CDR2 of SEQ ID NO: 9, and a light chain CDR3 of SEQ ID NO: 10.

In embodiments, the GCC-targeted therapeutic comprises a heavy chain that comprises an amino acid sequence of SEQ ID NO: 11, a light chain that comprises an amino acid sequence of SEQ ID NO: 12, or both. In embodiments, the GCC-targeted therapeutic agent is an antibody-drug conjugate. In embodiments, the antibody molecule is conjugated to monomethyl auristatin E (MMAE). In embodiments, the antibody-drug conjugate comprises a protease-cleavable linker. In embodiments, the antibody-drug conjugate comprises Formula I-5 herein, or a pharmaceutically acceptable salt thereof. In embodiments, the antibody-drug conjugate comprises Formula I-5 herein, or a pharmaceutically acceptable salt thereof, and Ab is an anti-GCC antibody molecule, or antigen binding fragment thereof, that comprises a heavy chain CDR1 of SEQ ID NO: 5, a heavy chain CDR2 of SEQ ID NO: 6, a heavy chain CDR3 of SEQ ID NO: 7, a light chain CDR1 of SEQ ID NO: 8, a light chain CDR2 of SEQ ID NO: 9, and a light chain CDR3 of SEQ ID NO: 10.

In embodiments, the GCC-targeted therapeutic agent is MLN0264. In embodiments, the disorder is cancer, e.g., gastric cancer, pancreatic cancer, colorectal cancer, esophageal cancer, cancer of the gastroesophageal junction, or lung cancer.

The present disclosure also provides, in certain aspects, a method of treating a disorder characterized by one or more GCC-expressing cells, comprising administering a therapeutically effective amount of a compound as described herein, e.g., a radiolabeled GCC-binding compound, e.g., a compound of Formula (VIa) to a patient in need thereof.

In embodiments, the disorder is cancer. In embodiments, the disorder is selected from solid tumor, a soft tissue tumor, a metastatic lesion, a sarcoma, an adenocarcinoma, or a carcinoma. In embodiments, the cancer is early or late stage cancer, or cancer of any of stages 0, 1, IIA, IIB, IIIA, IIIB, IIIC, and IV. In embodiments, the disorder is selected from colorectal cancer, gastric cancer, esophageal cancer, pancreatic cancer, lung cancer, leiomyosarcoma, rhabdomyosarcoma, and a neuroendocrine tumor, or any metastases thereof. In embodiments, the colorectal cancer is a colorectal adenocarcinoma, colorectal leiomyosarcoma, colorectal lymphoma, colorectal melanoma, or colorectal neuroendocrine tumor. In embodiments, the gastric cancer is a gastric adenocarcinoma, gastric sarcoma, or gastric lymphoma. In embodiments, the esophageal cancer is esophageal squamous cell carcinoma, or esophageal adenocarcinoma. In embodiments, the lung cancer is a squamous cell carcinoma or adenocarcinoma. In embodiments, the neuroendocrine tumor is a gastrointestinal or a bronchopulmonary neuroendocrine tumor.

In embodiments, the method further comprises administering an additional form of therapy to the patient. In embodiments, the additional form of therapy is radiation therapy. In embodiments, the additional form of therapy is a second therapeutic molecule. In embodiments, the second therapeutic molecule is a DNA-damaging agent. In embodiments, the DNA-damaging agent is selected from a topoisomerase I inhibitor, a topoisomerase II inhibitor, an alkylating agent, an alkylating-like agent, an athracylcine, a DNA intercalator, a DNA minor groove alkylating agent, and an antimetabolite. In embodiments, the DNA damaging agent is a topoisomerase I inhibitor selected from irinotecan, topotecan, and camptothecin. In embodiments, the DNA damaging agent is an alkylating-like agent selected from cisplatin, oxaliplatin, carboplatin, nedaplatin, satraplatin and triplatin. In embodiments, the DNA damaging agent is an antimetabolite selected from fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, and pemetrexed.

In some embodiments, the patient receives a dose of between about 50-100, 100-200, 200-500, 500-1000, 1000-2000, 2000-5000, or 5000-10000 μCi.

In some embodiments, the method further comprises administering a kidney protectant to the patient. In embodiments, the kidney protectant comprises one or more of Clinisol, lysine, lysine/arginine, Gelofusine, or amifostine. In certain embodiments, the kidney protectant is administered when the radioactive composition alone (e.g., the radiolabeled peptide) is administered in a dose that would result in significant nephrotoxicity in the absence of a kidney protectant. In some embodiments, the kidney protectant is administered when the radioactive composition alone (e.g., the radiolabeled peptide) is administered in a dose that would not result in significant nephrotoxicity in the absence of a kidney protectant.

In embodiments, the method results in a reduction in tumor growth fold change, measured from a baseline. The baseline may be, e.g., the tumor size before treatment begins. In embodiments, the method results in a higher probability of survival at a given time point, compared to an expected course of the disorder without treatment.

In certain aspects, the present disclosure also provides a use of a compound as described herein, e.g., a radiolabeled GCC-binding compound, e.g., compound of Formula (VI) comprising a radioactive atom, e.g., an alpha-emitter, for treating a disorder characterized by one or more GCC-expressing cells.

In certain aspects, the present disclosure also provides a use of the compound as described herein, e.g., a radiolabeled GCC-binding compound, e.g., compound of Formula (VI) comprising a radioactive atom, e.g., an alpha-emitter, in the preparation of a medicament for treating a disorder characterized by one or more GCC-expressing cells.

In certain aspects, the present disclosure also provides a method of radiolabeling a compound as described herein, e.g., a GCC-binding compound, e.g., compound of Formula (VI). The method may comprise providing a radiolabel, purifying the radiolabel, and contacting the compound with the purified radiolabel under conditions that allow for binding of the compound to the radiolabel. In embodiments, the method comprises one or more of, e.g., all of: (a) providing an amount of Gallium-68, (b) purifying the amount of Gallium-68, thereby producing purified Gallium-68, and (c) contacting about 45-65 ug of the compound with the purified Gallium-68, in a buffer, for an incubation time of about 3-20 minutes, at a temperature of about 60-100° C. and a pH of about 3.0-4.5; thereby producing a radiolabeled compound with a specific activity of about 25-33 MBq/nmol. In embodiments, the method comprises contacting about 45-65 ug of the compound with the purified Gallium-68, e.g., in a buffer. In embodiments, the method comprises incubating the compound and the purified radiolabel for an incubation time of about 3-20 minutes. In embodiments, the method comprises incubating the compound and the purified radiolabel at a temperature of about 60-100° C. In embodiments, the method comprises incubating the compound and the purified radiolabel at a pH of about 3.0-4.5

In embodiments, the method comprises generating an amount of 68Ga via a 68Ge/68Ga generator. In embodiments, the Gallium-68 is provided as Gallium-68 chloride. In embodiments, purifying the amount of Gallium-68 comprises one or more of eluting the Gallium-68 from the generator with HCl thereby producing an eluate, loading the eluate into a cation column, and eluting the Gallium-68 from the cation column using acetone and HCl.

In embodiments, the HCl used in eluting Gallium-68 from the generator is about 0.1M HCl, e.g., about 0.05-0.15M HCl or about 0.01-0.02M HCl.

In embodiments, the Gallium-68 is eluted from the cation column using one or more of acetone and HCl. The acetone may be, e.g., about 90-100%, 95-99%, or about 98% acetone. The HCl may be about, e.g., 0.02M HCl, 0.01-0.03M HCl, or 0.001-0.05M HCl.

In embodiments, step (c) involves about 20-70 μg of the compound. In embodiments, step (c) involves about 55 μg of the compound. In embodiments, the buffer comprises citrate, acetate, or phosphate. In embodiments, the buffer comprises sodium acetate. In embodiments, the temperature is about 100 degrees. In embodiments, the pH is about 3.75-4. In embodiments, the compound a compound according to Formula (VI). In embodiments, the incubation time is about 6-10 minutes. In embodiments, the method further comprises purifying the radiolabeled compound by buffer exchange. In embodiments, the method further comprises sterile-filtering the radiolabeled compound using a 0.2 μm filter.

In certain aspects, the present disclosure also provides a method of determining a suitable injection volume of a compound as described herein, e.g., a radiolabeled GCC-binding compound, e.g., a compound of Formula (VIa) to administer to a patient at a given time of injection, comprising computing the following formula: [injection dose volume]=[radioactive dose at the time of injection, in mCi]/{([Radioactive count at the time of calibration, in mCi]/[Volume of composition at the time of injection, in ml]×EXP(−6.14E-1 hours−1)*[time between calibration and injection, in hours]}.

In some aspects, the present disclosure provides a compound comprising:

(a) a peptide comprising
(i) an amino acid sequence of SEQ ID NO: 1,
(ii) an amino acid sequence at least 75% identical to SEQ ID NO:1 which comprises Ala at position 15 wherein the amino acids are numbered according to their position in native E. coli STp enterotoxin, or
(iii) an amino acid sequence that is at least 75% identical to SEQ ID NO: 1 wherein the peptide is 18 amino acids or less in length,

wherein the peptide has an amino terminus and has a free carboxy-terminus,

(b) a chelating moiety capable of binding a radioactive atom, e.g., a positron-emitting atom, e.g., a chelating moiety as described herein, wherein the chelating moiety comprises a macrocycle, e.g., a macrocycle comprising an O and/or a N, DOTA, NOTA, plurality of amines, EDTA, DTPA, TETA, DO3A, PCTA, or desferrioxamine, and

(c) a linker moiety that covalently attaches the amino-terminus of the amino acid sequence of the peptide to the chelating moiety, e.g., a linker moiety as described herein.

wherein the compound is capable of binding to guanylyl cyclase C (GCC).

In some embodiments, the compound is a compound of Formula (VI) or (VIa). In some embodiments, the compound is not a compound of Formula (VI) or (VIa).

In some embodiments, the linker moiety comprises an aminopentyl group, aminohexyl group, aminoheptyl group, phenyl group, or NCS moiety. In some embodiments, the linker moiety comprises (i) an aminopentyl group, aminohexyl group, or aminoheptyl group, (ii) a phenyl group, and (iii) a NCS moiety. In some embodiments, the linker moiety comprises (i) an aminohexyl group (ii) a phenyl group, and (iii) a NCS moiety.

In some aspect, the present disclosure provides a compound comprising:

(a) a peptide comprising an amino acid sequence at least 75% identical to SEQ ID NO:1 which comprises Ala at position 15 wherein the amino acids are numbered according to their position in native E. coli STp enterotoxin, wherein the peptide has an amino terminus and has a free carboxy-terminus,

(b) a chelating moiety capable of binding a radioactive atom, e.g., a positron-emitting atom, e.g., a chelating moiety as described herein, e.g., wherein the chelating moiety comprises a macrocycle, e.g., a macrocycle comprising an O and/or a N, DOTA, NOTA, plurality of amines, EDTA, DTPA, TETA, DO3A, PCTA, desferrioxamine, 4 or more N, or 4 or more carboxylic acid groups, or wherein the chelating moiety does not comprise S, and

(c) a linker moiety that covalently attaches the amino-terminus of the amino acid sequence of the peptide to the chelating moiety, e.g., a linker moiety as described herein.

wherein the compound is capable of binding to guanylyl cyclase C (GCC).

In some embodiments, the compound is a compound of Formula (VI) or (VIa). In some embodiments, the compound is not a compound of Formula (VI) or (VIa).

In some embodiments, the linker moiety comprises an aminopentyl group, aminohexyl group, aminoheptyl group, phenyl group, or NCS moiety. In some embodiments, the linker moiety comprises (i) an aminopentyl group, aminohexyl group, or aminoheptyl group, (ii) a phenyl group, and (iii) a NCS moiety. In some embodiments, the linker moiety comprises (i) an aminohexyl group (ii) a phenyl group, and (iii) a NCS moiety.

In some aspects, the present disclosure provides a compound comprising:

(a) a peptide comprising an amino acid sequence that is at least 75% identical to SEQ ID NO: 1 wherein the peptide is 18 amino acids or less in length, wherein the peptide has an amino terminus and has a free carboxy-terminus,

(b) a chelating moiety capable of binding a radioactive atom, e.g., a positron-emitting atom, e.g., a chelating moiety as described herein, e.g., wherein the chelating moiety comprises a macrocycle, e.g., a macrocycle comprising an O and/or a N, DOTA, NOTA, plurality of amines, EDTA, DTPA, TETA, DO3A, PCTA, desferrioxamine, 4 or more N, or 4 or more carboxylic acid groups, or wherein the chelating moiety does not comprise S, and

(c) a linker moiety that covalently attaches the amino-terminus of the amino acid sequence of the peptide to the chelating moiety, e.g., a linker moiety as described herein.

wherein the compound is capable of binding to guanylyl cyclase C (GCC).

In some embodiments, the compound is a compound of Formula (VI) or (VIa). In some embodiments, the compound is not a compound of Formula (VI) or (VIa).

In some embodiments, the linker moiety comprises an aminopentyl group, aminohexyl group, aminoheptyl group, phenyl group, or NCS moiety. In some embodiments, the linker moiety comprises (i) an aminopentyl group, aminohexyl group, or aminoheptyl group, (ii) a phenyl group, and (iii) a NCS moiety. In some embodiments, the linker moiety comprises (i) an aminohexyl group (ii) a phenyl group, and (iii) a NCS moiety.

In some aspects, the present disclosure provides a compound comprising:

(a) a peptide comprising
(i) an amino acid sequence of SEQ ID NO: 1,
(ii) an amino acid sequence at least 75% identical to SEQ ID NO:1 which comprises Ala at position 15 wherein the amino acids are numbered according to their position in native E. coli STp enterotoxin, or
(iii) an amino acid sequence that is at least 75% identical to SEQ ID NO: 1 wherein the peptide is 18 amino acids or less in length,
wherein the peptide has an amino terminus and has a free carboxy-terminus,

(b) a chelating moiety capable of binding a radioactive atom, e.g., a positron-emitting atom, and

(c) a linker moiety that covalently attaches the amino-terminus of the amino acid sequence of the peptide to the chelating moiety, wherein the linker moiety comprises an aminopentyl, aminohexyl, or aminoheptyl group,

wherein the compound is capable of binding to guanylyl cyclase C (GCC).

In some embodiments, the compound is a compound of Formula (VI) or (VIa). In some embodiments, the compound is not a compound of Formula (VI) or (VIa).

In some aspects, the present disclosure provides a compound comprising:

(a) a peptide comprising an amino acid sequence at least 75% identical to SEQ ID NO:1 which comprises Ala at position 15 wherein the amino acids are numbered according to their position in native E. coli STp enterotoxin, or wherein the peptide has an amino terminus and has a free carboxy-terminus,

(b) a chelating moiety capable of binding a radioactive atom, e.g., a positron-emitting atom, and

(c) a linker moiety that covalently attaches the amino-terminus of the amino acid sequence of the peptide to the chelating moiety, e.g., a linker moiety as described herein, e.g., wherein the linker moiety comprises an aminopentyl group, aminohexyl group, aminoheptyl group, phenyl group, or NCS moiety,

wherein the compound is capable of binding to guanylyl cyclase C (GCC).

In some embodiments, the compound is a compound of Formula (VI) or (VIa). In some embodiments, the compound is not a compound of Formula (VI) or (VIa).

In some aspects, the present disclosure provides a compound comprising:

(a) a peptide comprising an amino acid sequence that is at least 75% identical to SEQ ID NO: 1 wherein the peptide is 18 amino acids or less in length, wherein the peptide has an amino terminus and has a free carboxy-terminus,

(b) a chelating moiety capable of binding a radioactive atom, e.g., a positron-emitting atom, and

(c) a linker moiety that covalently attaches the amino-terminus of the amino acid sequence of the peptide to the chelating moiety, e.g., a linker moiety as described herein, e.g., wherein the linker moiety comprises an aminopentyl group, aminohexyl group, aminoheptyl group, phenyl group, or NCS moiety,

wherein the compound is capable of binding to guanylyl cyclase C (GCC).

In some embodiments, the compound is a compound of Formula (VI) or (VIa). In some embodiments, the compound is not a compound of Formula (VI) or (VIa).

In certain aspects, the present disclosure provides a compound comprising:

(a) a peptide comprising an amino acid sequence of SEQ ID NO: 1, wherein the amino acid sequence peptide has an amino terminus and has a free carboxy-terminus,

(b) a chelating moiety capable of binding a radioactive atom, e.g., a positron-emitting atom, and

(c) a linker moiety that covalently attaches the amino-terminus of the amino acid sequence of the peptide to the chelating moiety, and

(d) a positron-emitting atom, e.g., gallium-68,

wherein the compound is capable of binding to guanylyl cyclase C (GCC).

In some embodiments, the compound is a compound of Formula (VI) or (VIa). In some embodiments, the compound is not a compound of Formula (VI) or (VIa).

In some embodiments, the linker moiety comprises an aminopentyl group, aminohexyl group, aminoheptyl group, phenyl group, or NCS moiety. In some embodiments, the linker moiety comprises (i) an aminopentyl group, aminohexyl group, or aminoheptyl group, (ii) a phenyl group, and (iii) a NCS moiety. In some embodiments, the linker moiety comprises (i) an aminohexyl group (ii) a phenyl group, and (iii) a NCS moiety.

In certain aspects, the present disclosure provides a compound comprising:

(a) a peptide comprising an amino acid sequence of SEQ ID NO: 1 or an amino acid sequence at least 70%, 75%, 80%, 85%, 90%, 95%, or more identity to SEQ ID NO: 1, wherein the peptide does not comprise the sequence of native E. coli STp, e.g., the peptide (or the STp-like portion of the peptide) is 17 amino acids or less in length, e.g., the peptide (or the STp-like portion of the peptide) consists of SEQ ID NO: 1 or SEQ ID NO: 3, wherein the amino acid sequence peptide has an amino terminus and has a free carboxy-terminus,

(b) a chelating moiety capable of binding a radioactive atom, e.g., a positron-emitting atom, and

(c) a linker moiety that covalently attaches the amino-terminus of the amino acid sequence of the peptide to the chelating moiety, and

(d) a positron-emitting atom, e.g., gallium-68,

wherein the compound is capable of binding to guanylyl cyclase C (GCC).

In some embodiments, the compound is a compound of Formula (VI) or (VIa). In some embodiments, the compound is not a compound of Formula (VI) or (VIa).

In some embodiments, the linker moiety comprises an aminopentyl group, aminohexyl group, aminoheptyl group, phenyl group, or NCS moiety. In some embodiments, the linker moiety comprises (i) an aminopentyl group, aminohexyl group, or aminoheptyl group, (ii) a phenyl group, and (iii) a NCS moiety. In some embodiments, the linker moiety comprises (i) an aminohexyl group (ii) a phenyl group, and (iii) a NCS moiety.

The disclosure contemplates all combinations of any one or more of the foregoing aspects and/or embodiments, as well as combinations with any one or more of the embodiments set forth in the detailed description and examples.

All publications, patent applications, patents and other references mentioned herein are incorporated by references in their entirety.

Other features, objects, and advantages of the invention(s) disclosed herein will be apparent from the description and drawings, and from the claims.

### BRIEF DESCRIPTION OF THE DRAWINGS

FIG. 1. The ¹¹¹In-MLN6907 precursor can differentiate antigen density in nonclinical proof of concept studies. The left column shows light microscopy images of three cell types, with the highest expresser of GCC on top and the lowest expresser of GCC on the bottom. The second column from the left quantifies the expression level of GCC as determined by IHC. The next column of images shows 3D and 2D gamma camera images detecting Indium-111 when the compound adheres to GCC-producing cells.

FIG. 2 This figure depicts PET detection of [⁶⁸Ga]MLN6907 in a mouse model, with time points at 15, 45, and 60 minutes post-injection.

FIG. 3A-D. This figure shows the increase in solubility of DSP1 achieved by varying the conditions. FIG. 3A shows that increasing the percentage of ethanol increases solubility. FIG. 3B shows that increasing the pH increases solubility. FIG. 3C shows that heat and mixing each increases solubility. FIG. 3D shows the effects of various filters.

FIGS. 4A-D show the effect of different reaction conditions on the efficiency of labeling MLN6907. FIG. 4A shows labeling efficiency when the reaction time is varied, FIG. 4B shows the labeling efficiency when reaction temperature is varied, FIG. 4C shows the labeling efficiency when the reaction pH is varied. FIG. 4D shows the average specific activity at various pHs.

FIG. 5A-C. FIG. 5A shows the percent of the radiolabeled compound retained on various columns. FIG. 5B shows the effect of different eluents on elution efficiency of the radiolabeled compound from a Sep-Pak column. FIG. 5C shows the effects of different columns and eluents on elution efficiency of the radiolabeled compound from a Sep-Pak column.

FIG. 6. An exemplary calculation of a suitable volume of radioactive imaging compound to administer to a patient, based on the radioactive counts.

FIG. 7 shows the distribution of [⁶⁸Ga]MLN6907 in various tissues in a T84 tumor-bearing mouse model. [⁶⁸Ga]MLN6907 DP plus increasing concentrations of MLN6907 drug substance precursor (DSP) were administered as a single intravenous (IV) dose to female CB-17 SCID mice bearing T84 tumor xenografts. At 1 hour postdose, mice were euthanized and tissues were harvested, weighed, and assayed for 68Ga. The uptake of [⁶⁸Ga]MLN6907 DP was determined as percent injected dose per gram of tissue at 0.01-20-μg [⁶⁸Ga]MLN6907 DP±MLN6907 DSP mass injected. Values shown are mean±standard error of the mean (SEM).

FIG. 8 shows the distribution of [⁶⁸Ga]MLN6907 in various tissues in a HEK-293 GCC clone 2 tumor-bearing mouse model. [⁶⁸Ga]MLN6907 DP plus increasing concentrations of MLN6907 drug substance precursor DSP were administered as a single dose to female CB-17 SCID mice bearing HEK-293 GCC Clone 2 tumor xenografts. At 1 hour postdose, mice were euthanized and tissues were harvested, weighed, and assayed for 68Ga. The uptake of [⁶⁸Ga]MLN6907 DP was determined as percent injected dose per gram of tissue at 0.01-20-μg [⁶⁸Ga]MLN6907 DP±MLN6907 DSP mass injected. Values shown are mean±standard error of the mean (SEM).

FIG. 9 shows the distribution of [⁶⁸Ga]MLN6907 in various tissues in a PHTX-21C tumor-bearing mouse model. [⁶⁸Ga]MLN6907 DP plus increasing concentrations of MLN6907 drug substance precursor (DSP) was administered as a single dose to female CB-17 SCID mice bearing PHTX-21C primary xenografts. At 1 hour postdose, mice were euthanized and tissues were harvested, weighed, and assayed for 68Ga. The uptake of [⁶⁸Ga]MLN6907 DP was determined as percent injected dose per gram of tissue at 0.01-20 μg [⁶⁸Ga]MLN6907 DP±MLN6907 DSP mass injected. Values shown are mean±standard error of the mean (SEM).

FIG. 10 shows the distribution of [⁶⁸Ga]MLN6907 in various tissues in a PHTX-17C tumor-bearing mouse model. [⁶⁸Ga]MLN6907 DP plus increasing concentrations of MLN6907 drug substance precursor (DSP) was administered as a single dose to female CB-17 SCID mice bearing PHTX-17C primary xenografts. At 1 hour postdose, mice were euthanized and tissues were harvested, weighed, and assayed for ⁶⁸Ga. The uptake of [⁶⁸Ga]MLN6907 DP was determined as percent injected dose per gram of tissue at 0.01-20 μg-[⁶⁸Ga]MLN6907 DP±MLN6907 DSP mass injected. Values shown are mean±standard error of the mean (SEM).

FIG. 11. Summary of [⁶⁸Ga]MLN6907 DP and MLN6907 DSP biodistribution into tumor tissues.

FIG. 12. Increasing mass of [⁶⁸Ga]MLN6907 DP was administered as a single dose to female CB-17 SCID mice bearing HEK-293 GCC Clone 2 tumor xenografts. At 1 hour postdose, mice were euthanized and tissues were harvested, weighed, and assayed for 68Ga. The uptake of [⁶⁸Ga]MLN6907 DP was determined as percent injected dose per gram of tissue (% ID/g) at 0.005-0.5 μg [⁶⁸Ga]MLN6907 DP mass injected. Values shown are mean±standard error of the mean (SEM).

FIG. 13 depicts the results of a competitive binding study showing the K_{D} of MLN6907 for GCC on the surface of HEK-293 GCC clone 2 cells.

FIG. 14 shows the results of a study detecting the internalization rate of [¹¹¹In]MLN6907. The upper curve depicts the amount of intracellular MLN6907, while the lower curve depicts the amount of surface-bound MLN6907.

FIG. 15. The upper panel shows blood clearance of [⁶⁸Ga]MLN6907 in a T84 tumor bearing mouse model, compared to HEK 293 and HEK 293 GCC clone 2 cells. The y axis is the injected dose (% ID) per gram of tissue. In all three systems tested, blood clearance of the compound is rapid. The lower panel shows the amount of [⁶⁸Ga]MLN6907 uptake into different tumor tissues.

FIG. 16 shows the distribution of [⁶⁸Ga]MLN6907 in various tissues in a mouse model. The graph shows that [⁶⁸Ga]MLN6907 has a consistently low level in the blood and liver, indicating that there is little to no GCC in those tissues. This level is not further lowered by increasing amounts of unlabeled competitor (shown on the x axis), indicating that [⁶⁸Ga]MLN6907 does not bind to GCC in these tissues. In contrast, [⁶⁸Ga]MLN6907 accumulates in a tumor at high levels. The tumor-binding can be competed away by large injected doses of unlabeled peptide, indicating that the binding is specific. Levels of [⁶⁸Ga]MLN6907 in the kidney are high even in the presence of unlabeled competitor, indicating excreted through the kidneys, and that this excretion is not dependent on GCC receptors.

FIG. 17 shows the results of administering MLN6907 to mice bearing tumors with different GCC levels. Bₘₐₓ is the antigen surface density as determined by a competitive binding study using [⁶⁸Ga]MLN6907. The H-score is the staining intensity when GCC is detected by immunohistochemistry. In general, the Bₘₐₓ and H-scores correlate.

### DETAILED DESCRIPTION

### 1. Definitions

As used herein, “DSP” refers to the drug substance precursor, which is a compound described herein without a radiolabel. More specifically, DSP1 refers to a bulk, powder-form DSP, while DSP2 refers to formulation of DSP in solution. Exemplary DSPs include the compounds of formulas (II), (IV), (V) and (VI).

As used herein, “DP” stands for drug product, and refers to a radiolabeled compound described herein. Exemplary DPs include the compounds of formulas (I), (III), (IVa), (Va) and (VIa).

“Linker,” as used herein, is a moiety that connects two or more moieties together (e.g., a peptide that binds GCC and a chelating moiety. Linkers may include more than one functional group. For example, a linker may include a first functional group capable of reacting with a functional group on a moiety such as a peptide and a second functional group capable of reacting with a functional group on a second moiety such as a chelating moiety, e.g., a chelating moiety described herein.

The term “peptide,” as used herein, refers to a peptide comprising two or more amino acids but not more than 100 amino acids, covalently linked together through one or more amide bonds. In embodiments, upon administration of the peptide to a subject, the peptide binds to GCC. A peptide may comprise, e.g., more than three, four, five, six, seven, eight, nine, ten, eleven, twelve, thirteen, fourteen, fifteen, sixteen, seventeen, eighteen amino acids. In some embodiments, a peptide comprises more than 15, e.g., greater than 20, 25, 30, 35, 40, 45, 50, 55, 60, 65, 70, 75, 80, 85, or 90 amino acids. For example, in some embodiments, the peptide is more than 9, 10, 11, 12, 13, or 14 amino acids in length.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “colorectal cancer”, also commonly known as colon cancer or bowel cancer, refers to cancer from uncontrolled cell growth in the colon or rectum (parts of the large intestine), or in the appendix.

The terms “stomach cancer” and “gastric cancer” are used herein interchangeably.

As used herein, “GCC,” also known as “STAR”, “GUC2C”, “GUCY2C” or “ST receptor” protein refers to mammalian GCC, preferably human GCC protein. Human GCC refers to the protein shown in SEQ ID NO: 4 and naturally occurring allelic protein variants thereof. Other variants are known in the art. See, e.g., accession number Ensp0000261170, Ensembl Database, European Bioinformatics Institute and Wellcome Trust Sanger Institute, which has a leucine at residue 281; SEQ ID NO: 14 of published US patent application number 20060035852; or GenBank accession number AAB 19934. Typically, a naturally occurring allelic variant has an amino acid sequence at least 95%, 97% or 99% identical to the GCC sequence of SEQ ID NO: 4. The transcript encodes a protein product of 1073 amino acids, and is described in GenBank accession no.: NM_004963. GCC protein is characterized as a transmembrane cell surface receptor protein, and is believed to play a critical role in the maintenance of intestinal fluid, electrolyte homeostasis and cell proliferation.

Amino acid sequence for human GCC (GenPept Accession No. NP_004954; SEQ ID NO: 4):

| | |
|---|---|
| 1 | mktllldlal wsllfqpgwl sfssqvsqnc hngsyeisvl mmgnsafaep lknledavne |
| 61 | gleivrgrlq naglnvtvna tfmysdglih nsgdcrsstc egldllrkis naqrmgcvli |
| 121 | gpsctystfq myldtelsyp misagsfgls cdyketltrl msparklmyf lvnfwktndl |
| 181 | pfktyswsts yvykngtete dcfwylnale asysyfshel gfkvvlrqdk efqdilmdhn |
| 241 | rksnviimcg gpeflyklkg dravaedivi ilvdlfndqy fednvtapdy mknvlvltls |
| 301 | pgnsllnssf srnlsptkrd falaylngil lfghmlkifl engenittpk fahafrnltf |
| 361 | egydgpvtld dwgdvdstmv llytsvdtkk ykvlltydth vnktypvdms ptftwknskl |
| 421 | pnditgrgpq ilmiavftlt gavvllllva llmlrkyrkd yelrqkkwsh ippenifple |
| 481 | tnetnhvslk idddkrrdti qrlrqckydk krvilkdlkh ndgnftekqk ielnkllqid |
| 541 | yynitkfygt vkldtmifgv ieycergslr evlndtisyp dgtfmdwefk isvlydiakg |
| 601 | msylhsskte vhgrlkstnc vvdsrmvvki tdfgcnsilp pkkdlwtape hlrqanisqk |
| 661 | gdvysygiia qeiilrketf ytlscrdrne kifrvensng mkpfrpdlfl etaeekelev |
| 721 | yllvkncwee dpekrpdfkk iettlakifg lfhdqknesy mdtlirrlql ysrnlehlve |
| 781 | ertqlykaer dradrlnfml lprlvvkslk ekgfvepely eevtiyfsdi vgfttickys |
| 841 | tpmevvdmln diyksfdhiv dhhdvykvet igdaymvasg lpkrngnrha idiakmalei |
| 901 | lsfmgtfele hlpglpiwir igvhsgpcaa gvvgikmpry clfgdtvnta srmestglpl |
| 961 | rihvsgstia ilkrtecqfl yevrgetylk grgnettywl tgmkdqkfnl ptpptvenqq |
| 1021 | rlqaefsdmi anslqkrqaa girsqkprry asykkgtley lqlnttdkes tyf |

Calculations of “homology” or “identity” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 30%, 40%, or 50%, at least 60%, or at least 70%, 80%, 90%, 95%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. The percent homology between two amino acid sequences can be determined using any method known in the art. For example, the Needleman and Wunsch, J. Mol. Biol. 48:444-453 (1970), algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. The percent homology between two nucleotide sequences can also be determined using the GAP program in the GCG software package (Accelerys, Inc. San Diego, Calif.), using an NWSgapdna.CMP matrix and a gap weight of 40, 50, 60, 70, or 80 and a length weight of 1, 2, 3, 4, 5, or 6. An exemplary set of parameters for determination of homology are a Blossum 62 scoring matrix with a gap penalty of 12, a gap extend penalty of 4, and a frameshift gap penalty of 5.

An “anti-GCC antibody molecule” refers to an antibody molecule (i.e., an antibody, antigen-binding fragment of an antibody or antibody analog) which interacts with or recognizes, e.g., binds (e.g., binds specifically) to GCC, e.g., human GCC. Exemplary anti-GCC antibody molecules are summarized in Tables 3 and 4.

“Alkyl”, as used herein, refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. “Alkylene” refers to a double radical, that is, an aliphatic group substituted on two ends. In some embodiments, a straight chain or branched chain alkyl has 30 or fewer carbon atoms in its backbone (e.g., C1-C30 for straight chains, C3-C30 for branched chains), and in other embodiments can have 20 or fewer, or 10 or fewer. Likewise, certain cycloalkyls may have from 3-10 carbon atoms in their ring structure, and in some embodiments may have 5, 6 or 7 carbons in the ring structure. The term “alkenyl”, as used herein, refers to an aliphatic group containing at least one double bond; the term “alkynyl”, as used herein, refers to an aliphatic group containing at least one triple bond.

“Heteroalkylene” refers to an optionally substituted alkylene, which has one or more skeletal chain atoms selected from an atom other than carbon, e.g., oxygen, nitrogen, sulfur, phosphorus or combinations thereof. A numerical range may be given, e.g. C₁-C₆ heteroalkylene, which refers to the number of carbons in the chain, which in this example includes 1 to 6 carbon atoms. For example, a —CH₂OCH₂CH₂— radical refers to a “C₃” heteroalkylene. Connection to the molecule may be through either a heteroatom or a carbon in the heteroalkylene.

### 2. GCC-Targeting Compounds

This application provides GCC-targeting compounds that comprise a peptide, a chelating moiety capable of binding a radioactive atom, and a linker. This section describes each of these components, and compound comprising one or more of these components.

### 2.1 Peptides

This application discloses various GCC-targeting compounds that comprise a peptide as described herein. In some embodiments, the peptide comprises an amino acid sequence of SEQ ID NO: 1.

Peptides included in the compounds described herein are related to E. coli enterotoxin STp, an 18 amino acid sequence (SEQ ID NO: 2) that is a natural ligand for GCC. The Peptide, when included in a compound described herein, has an amino terminus, attached to a linker (L or L′), and a free carboxy-terminus.

In embodiments, the Peptide comprises the amino acid sequence: Cys²-Glu-Leu-Cys³-Cys⁴-Asn-Pro-Ala-Cys⁵-Ala-Gly-Cys⁶-Tyr (SEQ ID NO:1), or an amino acid sequence having at least 70%, 75%, 80%, 85%, 90%, 95%, or more identity with SEQ ID NO:1. Peptides comprising the amino acid sequence of SEQ ID NO:1, or having a percent identity with the amino acid sequence of SEQ ID NO:1, preferably have a disulfide bond between Cys² and Cys⁵ and a disulfide bond between Cys³ and Cys⁶.

In another embodiment, the Peptide comprises the amino acid sequence: Cys¹-Cys²-Glu-Leu-Cys³-Cys⁴-Asn-Pro-Ala-Cys⁵-Ala-Gly-Cys⁶-Tyr (SEQ ID NO:3), or an amino acid sequence having at least 70%, 75%, 80%, 85%, 90%, 95%, or more identity with SEQ ID NO:3. Peptides comprising the amino acid sequence of SEQ ID NO:3, or having a percent identity with the amino acid sequence of SEQ ID NO:3, preferably have a disulfide bond between Cys¹ and Cys⁴, a disulfide bond between Cys² and Cys⁵ and a disulfide bond between Cys³ and Cys⁶.

In another embodiment, the peptide comprises the amino acid sequence of STp1-18 (SEQ ID NO:2) or an amino acid sequence having at least 70%, 75%, 80%, 85%, 90%, 95%, or more identity with SEQ ID NO:2. Peptides comprising the amino acid sequence of SEQ ID NO:2, or having a percent identity with the amino acid sequence of SEQ ID NO:2, preferably have a disulfide bond between Cys⁵ and Cys¹⁰, Cys⁶ and Cys¹⁴ and a disulfide bond between Cys⁹ and Cys¹⁷, wherein the amino acids are numbered according to their position in native E. coli enterotoxin.

In some embodiments, the peptide does not comprise STh or a portion thereof. STh is an E. coli enterotoxin with certain sequence differences to STp. For example STp comprises an Ala at position 15 (numbered according to the native E. coli sequence), but STh comprises a Thr at the homologous position. Accordingly, in some embodiments, the peptide described herein comprises Ala at position 15 wherein the amino acids are numbered according to their position in native E. coli STp. In some embodiments, the peptide described herein does not comprise Thr at position 15 wherein the amino acids are numbered according to their position in native E. coli STp. As another example of a sequence difference, native STp is 18 amino acids long whereas native STh is 19 amino acids long. Accordingly, in some embodiments, the peptide described herein is 18 amino acids or less in length. In some embodiments, the peptide is less than or equal to 18, 17, 16, 15, or 14 amino acids in length.

In some embodiments, the peptide, or the STp-like portion of the peptide, is 13-18, 14-18, 15-18, 16-18, or 17-18 amino acids in length. In some embodiments, the peptide, or the STp-like portion of the peptide, is 13-17, 14-17, 15-17, or 16-17 amino acids in length. In some embodiments, the peptide, or the STp-like portion of the peptide, is 16-18 or 17-18 amino acids in length.

In some embodiments, a peptide, or the STp-like portion of the peptide, comprises more than 15, e.g., greater than 20, 25, 30, 35, 40, 45, 50, 55, 60, 65, 70, 75, 80, 85, or 90 amino acids. For example, in some embodiments, the peptide, or the STp-like portion of the peptide, is more than 9, 10, 11, 12, 13, or 14 amino acids in length.

In some embodiments, the peptide comprises an amino acid sequence that is at least 70%, 75%, 80%, 85%, 90%, 95%, or more identical to SEQ ID NO:1, which comprises Ala at position 15 wherein the amino acids are numbered according to their position in native E. coli STp enterotoxin. In some embodiments, the peptide comprises an amino acid sequence that is at least 70%, 75%, 80%, 85%, 90%, 95%, or more identical to SEQ ID NO:2, which comprises Ala at position 15 wherein the amino acids are numbered according to their position in native E. coli STp enterotoxin. In some embodiments, the peptide comprises an amino acid sequence that is at least 70%, 75%, 80%, 85%, 90%, 95%, or more identical to SEQ ID NO:3, which comprises Ala at position 15 wherein the amino acids are numbered according to their position in native E. coli STp enterotoxin.

In some embodiments, the peptide comprises an amino acid sequence that is at least 70%, 75%, 80%, 85%, 90%, 95%, or more identical to SEQ ID NO: 1 wherein the peptide is 18 amino acids or less in length. In some embodiments, the peptide comprises an amino acid sequence that is at least 70%, 75%, 80%, 85%, 90%, 95%, or more identical to SEQ ID NO: 1 wherein the peptide is 17 amino acids or less in length. In some embodiments, the peptide comprises an amino acid sequence that is at least 70%, 75%, 80%, 85%, 90%, 95%, or more identical to SEQ ID NO: 1 wherein the peptide is 16 amino acids or less in length. In some embodiments, the peptide comprises an amino acid sequence that is at least 70%, 75%, 80%, 85%, 90%, 95%, or more identical to SEQ ID NO: 1 wherein the peptide is 15 amino acids or less in length. In some embodiments, the peptide comprises an amino acid sequence that is at least 70%, 75%, 80%, 85%, 90%, 95%, or more identical to SEQ ID NO: 1 wherein the peptide is 14 amino acids or less in length.

A peptide included in the compound can have the amino acid sequence of SEQ ID NO:1, 2 or 3 with one, two, or three modifications (e.g., substitutions, insertions or deletions). For example, in one embodiment, the Peptide may have conservative or non-essential amino acid substitutions, which do not have a substantial effect on the binding activity of the Peptide. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The compounds described herein can include a Peptide that comprises (or consists of) the amino acid sequence of SEQ ID NO:1; a Peptide that comprises (or consists of) the amino acid sequence of SEQ ID NO:3; or a Peptide that comprises (or consists of) the amino acid sequence of SEQ ID NO:2.

In some embodiments, the peptide comprises an amino acid sequence of SEQ ID NO: 1, a first heterologous amino acid sequence at the N-terminus and/or a second heterologous amino acid sequence at the C-terminus. The heterologous amino acid sequence can be a sequence other than the corresponding sequence in native E. coli STp. For instance, the heterologous amino acid sequence can be an artificial sequence or a human sequence. In some embodiments, the first and/or second heterologous sequence is 1, 2, 3, 4, 5, 6, 7, 8, 9, or 10 amino acids in length. In some embodiments, the first heterologous sequence is at least 1, 2, 3, 4, 5, 6, 7, 8, 9, or 10 amino acids in length. In some embodiments, the first heterologous sequence is at most 1, 2, 3, 4, 5, 6, 7, 8, 9, or 10 amino acids in length. In some embodiments, the second heterologous sequence is at least 1, 2, 3, 4, 5, 6, 7, 8, 9, or 10 amino acids in length. In some embodiments, the second heterologous sequence is at most 1, 2, 3, 4, 5, 6, 7, 8, 9, or 10 amino acids in length.

A preferred GCC-binding Peptide called STp5-18, shown in SEQ ID NO: 3, is 14 amino acids long and comprises residues 5-18 of the full-length enterotoxin. This 14-amino acid fragment has nanomolar affinity for GCC.

In some embodiments, the peptide has one or more of the following properties:
a) the peptide binds to GCC at least as strongly as wild-type STp or STp5-18,
b) the peptide binds to GCC more strongly than wild-type STp,
c) the terminally modified peptide (e.g., modified on the N-terminus, e.g., comprising a chelating moiety and a linker at the N-terminus) binds to GCC at least as strongly as a corresponding peptide lacking a terminal modification, i.e., the peptide tolerates terminal modifications well;
d) the peptide has suitable distribution kinetics, e.g., has one or more of the kinetics parameters set out in Table 6 herein, e.g., has parameters that are within +/1 10%, 20%, 30%, 40%, or 50% of any one or more parameters of Table 6 herein;
e) the peptide comprises two or three disulfide bonds, e.g., wherein disulfide bonds connect two or more of Cys5 to Cys10, Cys6 to Cys14, and Cys9 to Csy17, wherein the amino acids are numbered according to their position in native E. coli enterotoxin, and in some embodiments the disulfide bonds give the peptide improved stability under labeling conditions relative to a peptide of the same sequence that lacks one or more disulfide bonds,
f) the labeled peptide has a high ratio of signal to background, e.g., a ratio that is as high as or higher than the signal:background ratio of full length STp,
g) the peptide has low toxicity at the levels of administration as a diagnostic or therapeutic, e.g., a TD50 (toxic dose in 50% of subjects) that is 10, 50, 100, 500, 1000, 5000, or 10000-fold higher than the ED50 or the dose at which the compound is typically administered as a diagnostic.

### 2.2 Chelating Moieties

In an embodiment, the Chelating moiety comprises one or more amines, ethers or carboxylic acids. In an embodiment, the Chelating moiety comprises a plurality of amines. In an embodiment, the Chelating moiety includes 4 or more N, 4 or more carboxylic acid groups, or a combination thereof.

In an embodiment, the Chelating moiety does not comprise S.

In one embodiment, the Chelating moiety comprises a ring. In embodiments, the ring comprises an O and/or an N. In an embodiment, the Chelating moiety is a ring that includes 3 or more N, 3 or more carboxylic acid groups, or a combination thereof.

In one embodiment, the Chelating moiety is a macrocycle. In some embodiments, the macrocycle comprises an O and/or an N. In an embodiment, the Chelating moiety is a macrocycle that includes 3 or more N, 3 or more carboxylic acid groups, or a combination thereof. In an embodiment, the macrocycle is a 12-membered ring.

Exemplary Chelating moieties include DOTA, NOTA, EDTA, DTPA, TETA, DO3A, PCTA, and desferrioxamine. Additional chelate structures include those described in WO2012/174136. In an embodiment, the Chelating moiety is DOTA.

### 2.3 Linkers

In general, a linker has a size and flexibility such that it allows attachment of a chelating moiety to a peptide so that the chelating moiety does not interfere with the function of the peptide, e.g., does not sterically block the peptide from binding to a receptor such as GCC.

In embodiments, the linker (L) comprises alkylene or heteroalkylene. In some embodiments, L comprises an alkylene or heteroalkylene attached to another alkylene or heteroalkylene through a functional group. Exemplary functional groups include a sulfide, a disulfide, an amide, an ester, a ketone, a urea, a thiourea, a carbamate, or a carbonate. In an embodiment, L is an amide, a urea or a thiourea. In an embodiment, L is a divalent linker described in WO2012/174136, for example, in paragraphs [0031]-[0035], the contents of which are incorporated herein by reference. In one embodiment, L is an aminopentyl, aminohexyl, or aminoheptyl group. In an embodiment, L comprises an aminohexyl group.

In an embodiment, L′ is alkylene, e.g., C₁-C₁₀ alkylene such as C₁, C₂, C₃, C₄, C₅, C₆, C₇, C₈, C₉, or C₁₀ alkylene.

In an embodiment, L comprises an alkanyl moeity, e.g., an unbranched alkanyl moeity, e.g., having at least 4, 5, or 6 carbons.

In some embodiments, the presence of an Ahx (aminohexyl) group in the linker leads to improved stability over the same linker lacking an Ahx group. For instance, the Ahx group can reduce a linker's tendency to self-cleave.

### 2.4 Radioactive Atoms

The radioactive atom can be, e.g., a positron emitter, an alpha emitter, a beta emitter and/or a gamma emitter. Exemplary radioactive atoms include ¹¹C, ¹³N, ¹⁵O, ¹⁸F, ⁶⁴Cu, ⁸²Ru, ⁷⁷Br, ¹²⁴I, ⁹⁹Tc, ⁷⁶Br, ⁷⁷Br, ⁶¹Cu, ⁶⁴Cu, ⁶⁷Ga, ¹¹¹In, ¹⁵³Gd, ¹²³I, ¹²⁵I, ¹³¹I, ²⁰¹Ti, ³²P, ²¹³Bi, ²²⁵Ac, ⁹⁰Y, and ¹⁷⁷Lu. In embodiments, the radioactive atom is ¹⁸F, ⁶⁸Ga, ⁸⁹Zr, or ⁶⁴Cu.

### 2.5 GCC-Targeting Compounds

GCC-targeting compounds useful, e.g., in the methods described herein, include the compounds of Formula (I):

Peptide-L-Chelating moiety-Radioactive Atom Formula (I)

wherein: Peptide is a peptide described herein; L is a divalent linker; the chelating moiety is a chelating moiety capable of binding a radioactive atom, and the radioactive atom is, e.g., a radioactive atom described herein.

In other embodiments, the GCC-targeting compound has the structure of formula (II):

Peptide-L-Chelating moiety Formula (II)

wherein: Peptide is a peptide described herein; L is a divalent linker; and the chelating moiety is a chelating moiety capable of binding a radioactive atom.

In embodiments, L comprises alkylene or heteroalkylene. In some embodiments, L comprises an alkylene or heteroalkylene attached to another alkylene or heteroalkylene through a functional group. Exemplary functional groups include a disulfide, an amide, an ester, a ketone, a urea, a thiourea, a carbamate, or a carbonate.

In embodiments, L is attached to Peptide or Chelating moiety using a functional group. In an embodiment, L is attached to both Peptide and Chelating moiety using a functional group. In embodiments, the functional group is an amide, an ester, a carbamate, a carbonate, a urea, a thiourea, a sulfide, or a di-sulfide. In an embodiment, L is an amide, a urea or a thiourea. In an embodiment, L is attached to Peptide using an amide. In an embodiment, L is attached to Peptide using an amide wherein the nitrogen of the amide is the terminal amine of the peptide. In an embodiment, L is attached to Chelating moiety using a urea or thiourea. In an embodiment, L is attached to Chelating moiety using a thiourea. In an embodiment, L is a divalent linker described in WO2012/174136, for example, in paragraphs [0031]-[0035], the contents of which are incorporated herein by reference. In one embodiment, L is an aminopentyl, aminohexyl, or aminoheptyl group.

In an embodiment, the Chelating moiety comprises one or more amines, ethers or carboxylic acids. In an embodiment, the Chelating moiety comprises a plurality of amines. In one embodiment, the Chelating moiety is a macrocycle, e.g., a macrocycle comprising an O and/or a N. Exemplary Chelating moieties include DOTA, NOTA, EDTA, DTPA, TETA, DO3A, PCTA, and desferrioxamine. Additional chelate structures include those described in WO2012/174136. In an embodiment, the Chelating moiety is DOTA.

The radioactive atom can be, e.g., a positron emitter, an alpha emitter and/or gamma emitter. Exemplary radioactive atoms include ¹¹C, ¹³N, ¹⁵O, ¹⁸F, ⁶⁴Cu, ⁸²Ru, ⁶⁸Ga, ¹²⁴I, ⁹⁹Tc, ⁷⁶Br, ⁷⁷Br, ⁶¹Cu, ⁶⁴Cu, ⁶⁷Ga, ¹¹¹In, ¹⁵³Gd, ¹²³I, ¹²⁵I, ¹³¹I, ²⁰¹Ti, ³²P, ²¹³Bi, ⁹⁰Y and ¹⁷⁷Lu. In embodiments, the radioactive atom is ¹⁸F, ⁶⁸Ga, ⁸⁹Zr, or ⁶⁴Cu.

Compounds, e.g., useful in the methods described herein, can have the structure of Formula (III):

Peptide-L-Chelating moiety-PET Formula (III)

wherein: Peptide is a peptide described herein; L is a linker; Chelating moiety is a chelating moiety described herein; and PET is a positron emitting atom.

In an embodiment, the compound comprises ⁶⁸Ga, e.g., DOTA chelated with ⁶⁸Ga.

In an embodiment, the compound is a compound of Formula (IV) or (IVa) below:

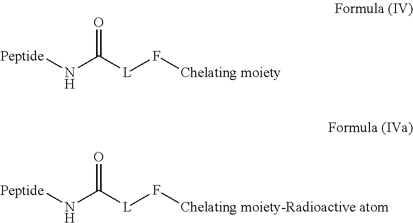

wherein Peptide, chelating moiety and Radioactive atom are as described above; L′ is an alkylene or heteroalkylene, and F is a functional group such as a functional group described above. In embodiments, Radioactive atom is PET, e.g., ¹⁸F, ⁶⁸Ga, ⁸⁹Zr, or ⁶⁴Cu, e.g., ⁶⁸Ga.

In an embodiment, F is a urea or thiourea. In an embodiment, F is a thiourea.

In an embodiment, L′ is alkylene, e.g., C₁-C₁₀ alkylene such as C₁, C₂, C₃, C₄, C₅, C₆, C₇, C₈, C₉, or C₁₀ alkylene.

In an embodiment, the compound is a compound of Formula (V) or (Va) below:

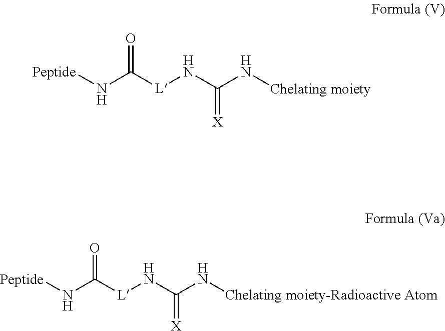

Wherein Peptide, Chelating moiety, Radioactive atom, and L′ are as described above, and X is O or S.

In embodiments, Radioactive atom is PET, e.g., ¹⁸F, ⁶⁸Ga, ⁸⁹Zr, or ⁶⁴Cu, e.g., ⁶⁸Ga.

In embodiments, X is S.

In embodiments, L is alkylene, e.g., C₁-C₁₀ alkylene such as C₁, C₂, C₃, C₄, C₅, C₆, C₇, C₈, C₉, or C₁₀ alkylene.

In embodiments, L is alkylene (e.g., C₅) and X is S.

In an embodiment, a compound described herein, e.g., the compound of Formula (III), (IVa) and/or (Va) has one or more of the following properties: high labeling efficiency, chelated incorporation of ⁶⁸Ga at greater than 60% (decay corrected), high overall yield: final purified compound >40% overall yield (decay corrected) based on starting ⁶⁸Ga ion; high specific activity: >500 Ci/mmol measured by HPLC; high stability: >90% labeled compound remaining up to 6 hours after preparation; high inhibitory potency (IC₅o<10 nM) for GCC; irreversible or slowly-reversible inhibition of target; high saline and ex vivo stability: >90% stable in saline and blood for up to 3 h at room temperature.

### 2.6 DSP Targeting Compounds

A compound described herein can be the drug substance precursor (DSP), which is a compound described herein without a radiolabel. For example, the compound can be a compound of Formula (II), Formula (IV), Formula (V) and/or Formula (VI). Such compounds can be in a composition, e.g., a composition which comprises a plurality of DSPs. In some embodiments, the composition can be a solution. In other embodiments, the composition is a dry powder. Various compositions are described in more detail below.

In one embodiment, the compound is MLN6907 DSP, a DSP according to Formula (VI):

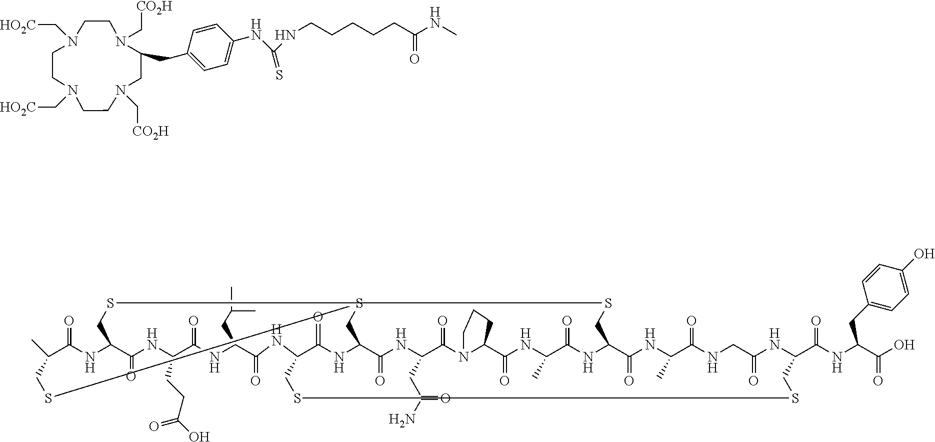 [SMILES: CNC(=O)CCCCCNC(=S)Nc1ccc(C[C@H]2CN(CC(=O)O)CCN(CC(=O)O)CCN(CC(=O)O)CCN2CC(=O)O)cc1 ; CC(C)C[C@@H]1NC(=O)[C@H](CCC(=O)O)NC(=O)[C@@H]2CSSC[C@@H]3NC(=O)[C@H](C)NC(=O)[C@@H]4CCCN4C(=O)[C@H](CC(N)=O)NC(=O)[C@H](CSSC[C@H](C)C(=O)N2)NC(=O)[C@H](CSSC[C@@H](C(=O)N[C@@H](Cc2ccc(O)cc2)C(=O)O)NC(=O)CNC(=O)[C@H](C)NC3=O)NC1=O]

### 2.7 DP Targeting Compounds

A compound described herein can be a drug product (DP), which refers to a radiolabeled compound described herein. For example, the compound can be a compound of Formula (I), Formula (III), Formula (IVa), Formula (Va) and Formula (VIa). In one embodiment, the compound is MLN6907 DP, a DP according to Formula (VIa):

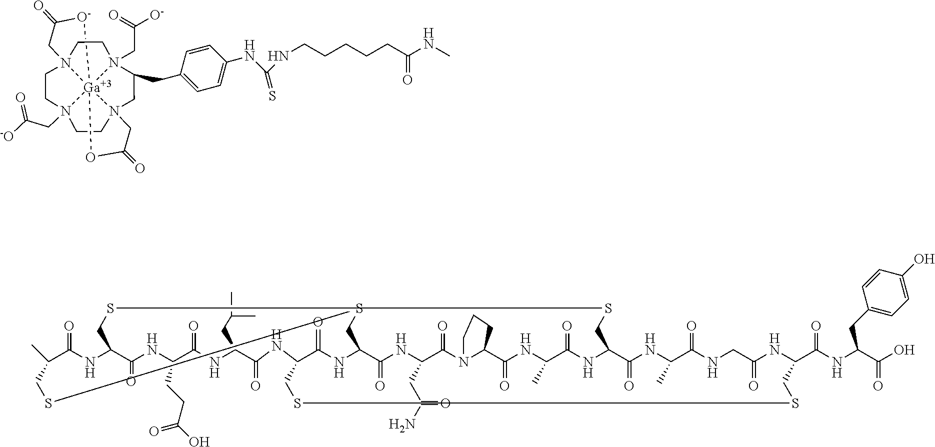

The Gallium-bound compound according to Formula (VIa) has a chemical formula of C₈₅H₁₁₉GaN₂₁O₂₈S₇, a mass of 2174.58, and a molecular weight of 2177.16. The [⁶⁸Ga]MLN6907 drug product consists of a chemically synthesized 14-amino acid truncated form of the porcine Escherichia coli heat-stable enterotoxin, STp(5-18), conjugated via a N′-benz-4-yl-N-(6-hexan)-1-oyl thiourea linker to a DOTA chelate bound to the radionuclide Gallium-68 (⁶⁸Ga). DOTA is a macrocyclic polydentate chelating agent. It is capable of chelating Ga⁺³ via its four nitrogen atoms and two of its O⁻ atoms. Gallium is a metallic radionuclide with a half-life of approximately 68 minutes. It decays by positron emission with an Emax=1,899-keV annihilation radiation. In some embodiments, the linker comprises a para benzyl group, a NCS moiety, and an aminohexyl moiety.

The DP compounds can be in a composition, e.g., a composition which comprises a plurality of DPs. In some embodiments, the composition comprises a plurality of compounds described herein, wherein at least one compound of the plurality is bound to a radioactive atom, e.g., a positron-emitting atom, e.g., Gallium-68, and at least one compound of the plurality is not bound to a radioactive atom. For example, at least one of the compounds of the plurality in the composition has a structure of Formula (I), e.g., a structure of Formula (III), and at least one of the compounds of the plurality in the composition has a structure of Formula (II). As another example, at least one of the compounds of the plurality in the composition has a structure of Formula (Va), and at least one of the compounds of the plurality in the composition has a structure of Formula (V). As another example, at least one of the compounds of the plurality has a structure of Formula (VIa), and at least one of the compounds of the plurality has a structure of Formula (VI).

In embodiments, the composition has a ratio of about 1:100 to 1:10,000 of DP compounds to unbound DSP compounds, e.g., a ratio of about 1:1,000 to 1:2,000 of DP compounds to unbound DSP compounds, e.g., a ratio of about 1:1,500 of DP compounds to DSP compounds.

### 3. Producing the GCC-Targeted Compounds

The compounds described herein can be made using methods known in the art, including solid or solution phase methods.

In one aspect, the disclosure provides solid phase synthetic methods for preparing a compound of the following Formula (Formula VI):

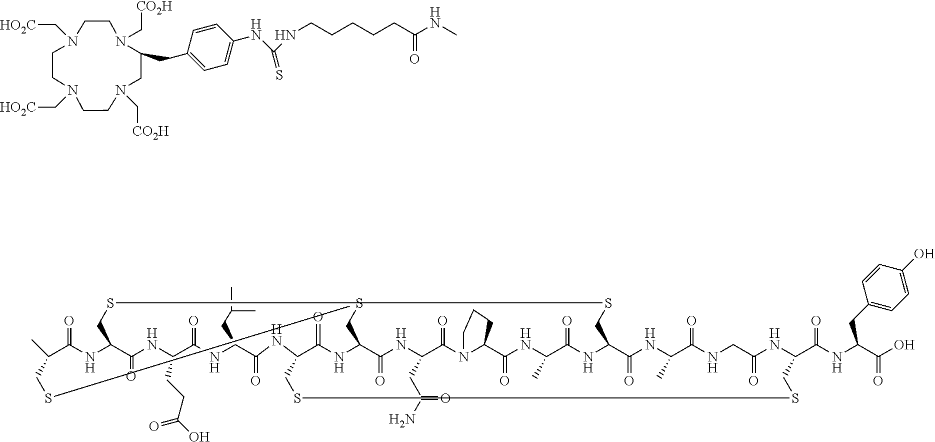 [SMILES: CNC(=O)CCCCCNC(=S)Nc1ccc(C[C@H]2CN(CC(=O)O)CCN(CC(=O)O)CCN(CC(=O)O)CCN2CC(=O)O)cc1 ; CC(C)C[C@@H]1NC(=O)[C@H](CCC(=O)O)NC(=O)[C@@H]2CSSC[C@@H]3NC(=O)[C@H](C)NC(=O)[C@@H]4CCCN4C(=O)[C@H](CC(N)=O)NC(=O)[C@H](CSSC[C@H](C)C(=O)N2)NC(=O)[C@H](CSSC[C@@H](C(=O)N[C@@H](Cc2ccc(O)cc2)C(=O)O)NC(=O)CNC(=O)[C@H](C)NC3=O)NC1=O]

The method includes preparing the peptide using solid phase peptide synthesis (e.g., using DIEA and DMF with a 2-chlorotritylchloride resin), subjecting the peptide to oxidation conditions so as to form the sulfide bonds as shown above, treating the resulting peptide with an activated acid of amino hexanoate (e.g., an acyl halide such as an acid chloride, an acyl azide, an acylimidazole, an anhydride, or an esteror succinamide) to form an amide bond; treating the resulting compound with thionyl chloride, after providing time for the thionyl chloride to react with the amine of the amino hexanoate, adding in protected DOTA (e.g., benzyl protected DOTA) or DOTA-NHS, deprotecting the DOTA to provide a compound of Formula (VI), and treating the compound with ⁶⁸Ga, thereby producing a compound of Formula (VIa). In some embodiments, the step of treating the compound of Formula (VI) with ⁶⁸Ga to provide a compound of Formula (VIa) is preformed in a different location and/or by a different party than the steps that provide a compound of Formula (VI).

In another embodiment, a compound of Formula (VI) can be prepared by treating the peptide (e.g., the peptide prepared as described above, e.g., using solid phase peptide synthesis followed by oxidation) with an activated acid of amino hexanoate as described above to form an amide bond which results in the peptide attached to the amino hexanoate. The amine of the amino hexanoate in the resulting compound is treated with benzyl protected DOTA, which is activated, for example with NCS, to form a thiourea bond between the DOTA and the amine of the amino hexanoate. The resulting compound is deprotected, e.g., removing the benzyl protecting groups (e.g., with Pt/C) to provide a compound of Formula (VI). The compound of Formula (VI) is then treated with ⁶⁸Ga, to provide a loaded compound of Formula (VIa). In some embodiments, the step of treating the compound of Formula (VI) with ⁶⁸Ga to provide a compound of Formula (VIa) is preformed in a different location and/or by a different party than the steps that provide a compound of Formula (VI).

The compound, e.g., a DSP compound described herein, can be assayed by LC-MS. For an MLN6907 DSP1, a m/z peak of 1055.6 Da (mass 2110.2 Da) is expected from the intact peptide conjugated to DOTA via the linker.

### 3.1 Methods of Producing the DSP2 Composition

To produce a soluble formulation of a DSP compound described in the previous section, the following exemplary protocol can be used. The powdered compound can be dissolved in NaOAc at a pH of about 7.0-7.5, e.g., 7.3. Sucrose, ethanol and acetic acid can then be added. The final pH of the mixture can be about 3.5 to 4.0, e.g., 3.8.

In some embodiments, the soluble formulation comprises the GCC-targeting compound and one or more of: a buffer (e.g., NaOAc/Acetic acid), a solubilizer (e.g., ethanol), a lyoprotectant (e.g., sucrose), polysorbate 80, and Pluronic F-68. The GCC-targeting compound can be present at 25-100 ug/mL. The NaOAc and acetic acid can be added as 0.05 and 0.2M solutions. Ethanol can be present at 0-25%. Sucrose can be present at 0-2%. Polysorbate 80 can be present at about 0.01%. Pluronic F-68 can be present at about 0.2 mM.

An exemplary formulation is provided in Table 1 below:

| Ingredients | Quantity |
|---|---|
| MLN6907 DSP1 | 25-30 μg/mL, e.g., 27.5 μg/mL |
| Sodium acetate, trihydrate | 0.01-0.05M, e.g., 0.02M |
| Glacial acetic acid | 0.1-0.2M, e.g., 0.18M |
| Ethanol | 5-15%, e.g., 10% |
| Sucrose | 0.5-2%, e.g., 1% |

### 3.2 Methods of Radiolabeling the GCC-Targeting Compounds

The GCC-targeting compounds described herein can be radiolabeled, e.g., according to the following exemplary protocol. First, a 68Ge/68Ge generator can be used to generate Gallium-68, which can be purified and concentrated. The Gallium-68 is then combined with a DSP2 solution described herein under conditions that allow for labeling to occur. Finally, the reaction product is purified. Each step is discussed in more detail below.

A ⁶⁸Ga generator stores a parent isotope, Germanium-68, waiting for a fraction of the Germanium-68 to decay into Gallium-68, and then purifying the Gallium-68 away from the parent isotope. One suitable ⁶⁸Ga generator is manufactured Eckert & Ziegler. The parent ⁶⁸Ge is primarily produced in a proton accelerator by (p, 2n) reaction on a Ga₂O₃ target at approximately 23 MeV proton energy. The reaction is: Ga-69+p->2n+Ge-68. Other options are ^{nat}Ga(p, xn) processing by ⁶⁹Ga(p, 2n)+⁷¹Ga(p, 4n) reactions at higher proton energies, and ⁶⁶Zn(α, 2n).

A ⁶⁸Ge/⁶⁸Ga generator can be a closed system consisting of a chromatographic column made from radiation-resistant material such as quartz glass and thermoplastic which is packed with sorbent material. The sorbent, in which ⁶⁸Ge is strongly absorbed, is generally a solid support such as metal oxides (Al₂O₃, TiO₂ or SnO₂), organics, and inorganic supports. A stock acidic ⁶⁸Ge solution is applied to the column to immobilize the ⁶⁸Ge parent nuclide on the sorbent. ⁶⁸Ga is continuously produced by decay of its radioactive parent, ⁶⁸Ge, and can be eluted with dilute HCl.

Several options are available for purifying and concentrating the Gallium-68. For instance, an acetone method can be used; this method removes ⁶⁸Ge and other contaminating heavy metals using a cation purification step. It is also possible to fractionate the Gallium-68 to purify and concentrate it. One can also elute the Gallium-68 from the generator using, e.g., HCl, e.g., 0.1M HCl, thereby producing an eluate, load the eluate into a cation column such as an SCX column, and elute the Gallium-68 from the cation column, e.g., using 98% acetone and 0.02M HCl.

The Gallium cleanup reaction can be performed in a single-use cassette. For instance, a semi-closed automated system (Eckert & Ziegler Modular-Lab PharmTracer), utilizing single-use, sterile, disposable cassettes, can be used. These cassettes are assembled under GMP-compliant clean room conditions, sterilized with gamma radiation and double vacuum-packed.

The labeling reaction is carried out under conditions selected to achieve a labeling efficiency high enough to reliably detect the labeled product in a subject's body. Exemplary suitable conditions are given here. The reaction time can be 3-10 minutes, for instance 10 minutes. The temperature can be 60-100° C. or 80-100° C., for instance 100° C. The pH can be 3.0-4.5, for instance 3.8; the pH should be kept low enough to avoid the formation of ⁶⁸Ga oxide and hydroxide species (colloid) which sometimes occurs above pH 4, but high enough to deprotonate the DOTA chelator. The reaction buffer can be selected to achieve the desired pH. Suitable reaction buffers include citrate, acetate, and phosphate buffers, for instance 0.2M NaOAc. The mass of compound added to the reaction can be 20-70 ug, about 30-60 ug, about 40-50 ug, or about 55 ug. Acetone from the reaction vial can be evaporated. The mixture can then cooled, and saline added.

In some embodiments, the labeling reaction is automated, for instance by the Modular-Lab PharmTracer system. The graphical interface of the Modular-Lab PharmTracer can be used to control the synthesis process. Parameters such as temperature, activity, radio-detector readings, flow rates, pressure and valve settings can be controlled and monitored. Reports containing relevant data and information can be created after each run. In some embodiments, the labeling protocol is locked and cannot be changed by the user.

After the labeling reaction is complete, the reaction product can be purified. The purification process can comprise a buffer exchange step that removes any residual acetone. The product can be applied, e.g., to a tC18 SepPak cartridge and then eluted using a solution of ethanol and water, for instance 1.5 mL of 45% ethanol. It can then be diluted with NaCl. As an additional purification step, the product can be sterile-filtered through one or more 0.2 μm filters, such as a PVDF filter. In some embodiments, the product is filtered through two, three, or more sequential filters.

The labeled compound obtained by this process has a specific activity of between 26 and 23 MBq/nmol, such as about 29.5 MBq/nmol, and may have a radiochemical purity of about 99.94-99.98%, such as about 99.97%.

The radiolabeled compound, suitable for administration to patients, can have a pH of about 5.5, e.g., pH 5-6, e.g., pH 45.-6.5, e.g., pH 4-7.

### 4. Pharmaceutical Compositions Comprising GCC-Targeting Compounds

Also included are containers comprising a compound or composition described herein, e.g., a DP compound or composition. In embodiments, a DP composition should have enough of the GCC-targeting compound to administer to a patient, plus an additional amount for assaying. For instance, the container may be a vial with a total volume of about 9.5 ml. In some embodiments, the compound (e.g., [⁶⁸Ga]MLN6907) is manufactured by radiolabeling nominal 55 μg of a DSP compound (e.g., a MLN6907 DSP2 compound) with ⁶⁸Ga chloride. The majority of the vial contents (up to 7.5 mL) can be used for patient dosing; however, approximately 2 mL of the DP can be used for analytical testing or is overage.

In one embodiment, the composition comprises a DP compound and a pharmaceutically acceptable carrier, e.g., an eluent and/or a diluents. An exemplary drug product formulation for the GCC-binding compound is set out in Table 2 below:

| Ingredient | Amount per Vial | Function |
|---|---|---|
| [⁶⁸Ga]MLN6907 DP^{b} | ≧12.73 mCi | Imaging Agent |
| Ethanol | ≦8% | Eluent |
| Sodium Chloride | <0.9% | Diluent |

^{b}The [⁶⁸Ga]MLN6907 DP is manufactured by radiolabeling 55 μg MLN6907 peptide-DOTA conjugate with 68Ga chloride obtained from a 68Ge/68Ga generator and filtered using a 0.2 μm filter. A patient dose may be 4.0 to 6.0 (+10%) mCi of [⁶⁸Ga]MLN6907 DP.
^{c} Saline (0.9% sodium chloride) is used as a diluent for the product. The saline is diluted slightly with the addition of ethanol.

In some embodiments, the percentage of ethanol in the radiolabeled compound suitable for administration to a patient is between 3.3 and 3.8%.

### 5. Kits Comprising GCC-Targeted Compounds

Also within the scope of the invention are kits comprising a compound or composition as described herein. The kit can include one or more other elements including: instructions for use; other reagents, e.g., a label, a therapeutic agent, or an agent useful for chelating, or otherwise coupling, an DSP compound to a label or therapeutic agent, or a radioprotective composition; devices or other materials for preparing the compound or composition for administration; pharmaceutically acceptable carriers; and devices or other materials for administration to a subject. Instructions for use can include instructions for diagnostic applications of the compound or composition to detect GCC, in vitro, e.g., in a sample, e.g., a biopsy or cells from a patient having a cancer, or in vivo. For example, when the GCC-targeted compound is a medical imaging compound, it may be provided with instructions for use in imaging or instructions for labeling the DSP with a detectable radiolabel. The instructions can include guidance for therapeutic application including suggested dosages and/or modes of administration, e.g., in a patient with a cancer (e.g., a cancer of gastrointestinal origin, such as, for example, colon cancer, stomach cancer, esophageal cancer). Other instructions can include instructions on coupling of the compound, e.g., a DSP compound to a radiolabel or a therapeutic agent, or for purification of a labeled compound, e.g., from unreacted conjugation components. As discussed above, the kit can include a label, e.g., any of the labels described herein. As discussed above, the kit can include a therapeutic agent, e.g., a therapeutic agent described herein. In some applications the compound will be reacted with other components, e.g., a radiolabel or therapeutic agent, e.g., a radioisotope, e.g., yttrium or lutetium. In such cases the kit can include one or more of a reaction vessel to carry out the reaction or a separation device, e.g., a chromatographic column, for use in separating the finished product from starting materials or reaction intermediates. In certain embodiments, the GCC-targeting compound is provided together with a cassette useful for preparing the compound for administration to a subject. The cassette may be used for the radiolabeling process, for instance for a step of purifying the radionuclide, e.g., Gallium-68, obtained from a generator, or for a step of contacting the DSP with the radionuclide, e.g., Gallium-68. In some embodiments, the kit comprises a device for administering the GCC-targeted compound, such as a pre-loaded syringe, optionally with radiation shielding. In some embodiments, the kit comprises a device or reagent for assaying the GCC-targeted compound.

The kit can further contain at least one additional reagent, such as a diagnostic or therapeutic agent, e.g., a diagnostic or therapeutic agent as described herein, and/or one or more additional anti-GCC antibody molecules or immunoconjugates, formulated as appropriate, in one or more separate pharmaceutical preparations.

The kit can further contain a radioprotectant. The radiolytic nature of isotopes is known. In order to overcome this radiolysis, radioprotectants may be included, e.g., in the reaction buffer, as long as such radioprotectants are benign, meaning that they do not inhibit or otherwise adversely affect the labeling reaction, e.g., of an isotope, to the compound, e.g., the DSP compound. The formulation buffer of the present invention may include a radioprotectant such as human serum albumin (HSA) or ascorbate. Other radioprotectants are known in the art and can also be used in the formulation buffer, i.e., free radical scavengers (phenol, sulfites, glutathione, cysteine, gentisic acid, nicotinic acid, ascorbyl palmitate, HOP(:O)H₂I glycerol, sodium formaldehyde sulfoxylate, Na₂S₂O, Na₂S₂O₃, and SO₂, etc.).

A provided kit is useful for radiolabeling a peptide with a radioisotope for administration to a patient. The kit includes (i) a vial containing a radiolabeled compound or composition, (ii) a vial containing formulation buffer for stabilizing and administering the radiolabeled compound or composition to a patient, and (iii) instructions for performing the radiolabeling procedure. The kit provides for exposing a compound or composition described herein to the radioisotope or a salt thereof for a sufficient amount of time under amiable conditions, e.g., as recommended in the instructions. A radiolabeled antibody having sufficient purity, specific activity and binding specificity is produced. The radiolabeled compound or composition may be diluted to an appropriate concentration, e.g., in formulation buffer, and administered directly to the patient with or without further purification. The compound or composition, e.g., a DSP compound or composition may be supplied as a dry powder or in solution, e.g., as described herein.

### 6. Dosing of GCC-Targeted Imaging Compounds

The compounds and compositions described herein can be administered to a subject in an amount sufficient, e.g., to provide good resolution of GCC expression, e.g., in the subject. For example, a dose below 100 ug/person is predicted to provide good resolution of GCC expression levels, without saturation. This dose was calculated by pharmacokinetic modeling, as described in more detail in the Examples. However, an even lower dose can produce good image resolution while further improving the safety profile of the compound or composition. Accordingly, in some embodiments, the mass dose administered to a subject is about 30-55 μg, about 35-50 μg, about 40-45 μg, or about 43.4 μg.

The dose should have an amount of radioactivity sufficient to produce a good quality image, while maintaining a good safety profile. In some embodiments, the dose administered to a subject is 2.0-10.0 mCi, 3.0-7.0 mCi, 3.6-6.6 mCi, or 4.0-6.0 mCi.

Because Gallium-68 decays with an approximate 68 minute half-life, the GCC-binding imaging composition should be administered to a subject shortly enough after production of the Gallium-68 that there is still detectable positron emission. A suitable length of time is less than 3 hours, e.g., 1.5 hours, for instance 1 hour after labeling.

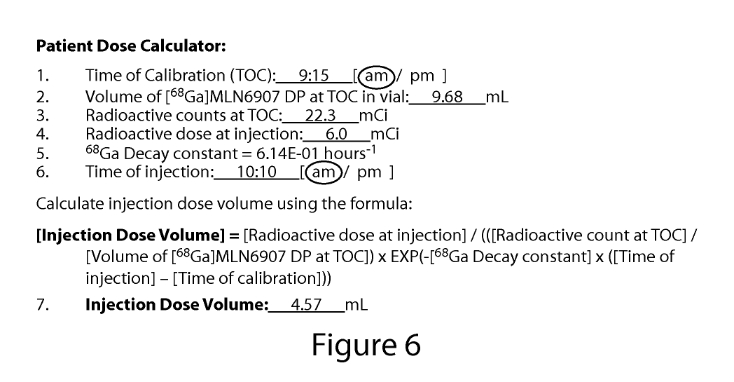

In some embodiments, the subject is administered a volume of the formulation that corresponds to the desired mass dose and desired level of positron emission. The formulation of [68Ga]MLN6907 DP to be administered for imaging can be 0.0058 mg/mL in saline with ≦8% ethanol. A suitable volume dose for a subject can be calculated as shown in FIG. 6. FIG. 6 shows use of the following formula to determine the correct volume of labeled compound to inject into a subject for PET imaging:

[injection dose volume]=[radioactive dose at the time of injection, in mCi]/{([Radioactive count at the time of calibration, in mCi]/[Volume of composition at the time of injection, in ml]×EXP(−6.14E-1 hours−1)*[time between calibration and injection, in hours]}

### 7. Routes of Administration

When administered as an imaging agent, the compounds and compositions described herein will typically be delivered intravenously. Systemic intravenous administration of the compound will have no or substantially no effect on intestinal GCC receptors (GCC+ epithelial tissues are anatomically inaccessible), while having access to extra-intestinal GCC expressing tumor cells irrespective of genotype, including primary or metastatic colorectal tumors, esophageal tumors, stomach tumors, adenocarcinoma at the gastroesophageal junction, tumors of the small intestine, pancreatic tumors, lung tumors (e.g., squamous cell carcinoma, adenosquamous carcinoma, adenocarcinoma), neuroendocrine tumors, neuroectodermal tumors, and soft tissue sarcomas (e.g., leiomyosarcomas and rhabdomyosarcomas). Any other mode of administration that allows the compound to reach the tumor(s) is acceptable. Therefore, in some embodiments, the compound or composition is administered as intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrastemal injection and infusion.

When administered as a therapeutic agent, the compound will typically be delivered intravenously. Other potential routes of administration include intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrastemal injection and infusion.

### 8. Diagnostic Methods

The compounds and compositions comprising the compounds described herein can be used for in vitro, ex vivo and in vivo detection of GCC-expressing cells. For example, the compounds and compositions described herein can be administered to a patient in amounts suitable for in vivo imaging of GCC-expressing cells, e.g., GCC-expressing cancer cells, and, e.g., can distinguish between GCC-expressing cancer cells and normal tissue. Cancerous cells that express GCC include, e.g., cells from a cancer of the gastrointestinal origin (e.g., colorectal cancer, stomach cancer, small intestine cancer, or esophageal cancer), pancreatic cancer, lung cancer (e.g., squamous cell carcinoma, adenosquamous carcinoma, adenocarcinoma), soft-tissue sarcomas such as leiosacroma or rhabdomyosarcoma, gastrointestinal or bronchopulmonary neuroendocrine tumors, or neuroectodermal tumors, or any metastatic lesions thereof.

The compounds and compositions described herein can be used to detect and quantify GCC-expressing cells associated with cancerous disorders such as solid tumors, soft tissue tumors, and metastatic lesions. Examples of solid tumors that express GCC include malignancies, e.g., sarcomas, adenocarcinomas, and carcinomas, of the various organ systems, such as those of gastrointestinal origin including but not limited to primary or metastatic colorectal cancer (e.g., colorectal adenocarcinoma, colorectal leiomyosarcoma, colorectal lymphoma, colorectal melanoma, or a colorectal neuroendocrine tumor), primary or metastatic stomach cancer (e.g., gastric adenocarcinoma, lymphoma, or sarcoma), primary or metastatic esophageal cancer (e.g., a squamous cell carcinoma or adenocarcinoma of the esophagus), primary or metastatic cancer of the gastroesophageal junction, and primary or metastatic small intestine cancer. Several different types of cancer of non-gastrointestinal origin have been shown to express GCC, including but not limited to primary or metastatic pancreatic cancer, primary or metastatic lung cancer (e.g., squamous cell carcinoma or adenocarcinoma), primary or metastatic soft-tissue sarcomas (e.g., leiomyosarcomas and rhabdomyosarcomas), primary or metastatic neuroendocrine tumors (e.g., gastrointestinal or bronchopulmonary neuroendocrine tumors), and primary or metastatic neuroectodermal tumors.

In one embodiment, the disclosure features methods for ex vivo or in vitro detection of GCC-expressing cells, e.g., GCC-expressing cancerous cells, the method comprising contacting a sample, e.g., a biopsy sample, with a compound or composition described herein.

In another embodiment, the disclosure features a method for in vivo detection of GCC-expressing cells, e.g., GCC-expressing cancerous cells, in a patient, the method comprising administering an effective amount of a compound or composition described herein to the patient, and detecting the binding of the compound to GCC-expressing cells in the patient. Also provided is a method comprising administering an effective amount of a compound or composition described herein to the patient, and detecting the location of the compound in the patient's body. This detection can be accomplished via a detectable label that is part of the compound. Also provided is a method of (i) administering to a subject (e.g., a patient having a cancer) a GCC-targeted compound (e.g., MLN6907), conjugated to a detectable label or marker; (ii) exposing the subject to a means for detecting said detectable label or marker to the GCC-expressing tissues or cells.

In some embodiments, the pharmaceutical composition administered to the subject comprises a pharmaceutically acceptable carrier or diluent and a conjugated compound that comprises a GCC-targeted moiety and an active moiety wherein the active moiety is a radioactive agent and the GCC-targeted moiety is E. coli enterotoxin, a fragment thereof, or a derivative thereof.

The compounds and compositions described herein can be used in a method of imaging and detecting of a GCC-associated disorder, e.g., cancer, e.g., a cancer described herein, performed by using gamma imaging, magnetic resonance imaging, magnetic resonance spectroscopy or fluorescence spectroscopy.

Diagnostic techniques described herein can use radioactive tracers which emit positrons at a particular site or sites in the patient's body. When a radionuclide emits a positron, the positron quickly collides with an electron, annihilating both particles and producing a pair of gamma rays. The gamma rays can then be detected, e.g., with a PET scanner. More broadly, when the compound is labeled with a radioactive label, it can be detected by any device capable of detecting the radioactivity. These radioactive atoms are generally short-lived isotopes linked to compounds. They can be given, e.g., by injection, inhalation or orally. In one technique, single photons are detected by a gamma camera which can view organs from many different angles. The camera builds up an image from the points from which radiation is emitted; this image is enhanced by a computer and viewed by a physician on a monitor for indications of abnormal conditions. Exemplary methods include positron emission tomography (PET) and single photon emission computed tomography (SPECT).

PET is a technique that indirectly detects isotopes. The isotopes can be produced in a cyclotron or other radionuclide generator. A positron-emitting radionuclide is introduced, e.g., by injection, and accumulates in the target tissue. As it decays it emits a positron, which combines with a nearby electron resulting in the emission of two identifiable gamma rays in opposite directions. These can be detected using a gamma camera. Radioactive atoms useful for PET imaging, include, e.g., ¹¹C, ¹³N, ¹⁵O, ¹⁸F, ⁶⁴Cu, ⁸²Ru, ⁶⁸Ga and ¹²⁴I. PET imaging is often combined with CT imaging; CT imaging uses X-rays to generate a three-dimensional image of the patient's body. PET techniques are described in more detail in the section below.

SPECT imaging uses a gamma camera to detect gamma rays. Radioactive atoms useful for SPECT imaging include, e.g., ⁹⁹Tc, ⁷⁶Br, ⁷⁷Br, ⁶¹Cu, ⁶⁷Ga, ¹¹¹In, ¹⁵³Gd, ¹²³I, ¹²⁵I, ¹³¹I, ²⁰¹Ti and ³²P. SPECT imaging is often combined with CT imaging.

Other radioactive atoms useful, e.g., in the PET and SPECT methods, are known in the art and described herein.

Examples of labels useful for diagnostic imaging in accordance with the methods described herein are radiolabels such as ³²P, ³H, ¹⁴C, ¹⁸⁸Rh, ⁴³K, ⁵²Fe, ⁵⁷Co, ⁶⁷Cu, ⁶⁷Ga, ⁶⁸Ga, ⁷⁷Br, ⁸¹Rb/^{81M}Kr, ^{87M}Sr, ⁹⁹Tc, ¹¹¹In, ¹¹³M In, ¹²³I, ¹²⁵I, ¹²⁷Cs, ¹²⁹Cs, ¹³¹I, ¹³²I, ¹⁹⁷Hg, ²⁰³Pb and ²⁰⁶Bi, and ²¹³Bi; fluorescent labels such as fluorescein and rhodamine; nuclear magnetic resonance active labels; positron emitting isotopes of oxygen, nitrogen, iron, carbon, or gallium (e.g., ⁶⁸Ga, ¹⁸F) detectable by a single photon emission computed tomography (“SPECT”) detector or positron emission tomography (“PET”) scanner; chemiluminescers such as luciferin; and enzymatic markers such as peroxidase or phosphatase. Short-range radiation emitters, such as isotopes detectable by short-range detector probes, can also be employed. Imaging can also be performed, for example, by radioscintigraphy, nuclear magnetic resonance imaging (MRI) or computed tomography (CT scan). Imaging by CT scan may employ a heavy metal such as iron chelates. MRI scanning may employ chelates of gadolinium or manganese.

The GCC-targeted compound can be labeled with such reagents using any suitable protocol, and numerous protocols are known in the art. One preferred label is Gallium-68, and labeling methods are described, e.g., in Example 3 below. Other labels and methods of labeling are described in, e.g., Magerstadt, M. (1991) Antibody Conjugates And Malignant Disease, CRC Press, Boca Raton, Fla.; and Barchel, S. W. and Rhodes, B. H., (1983) Radioimaging and Radiotherapy, Elsevier, NY, N.Y., each of which is incorporated herein by reference, teach the conjugation of various therapeutic and diagnostic radionuclides to polypeptides. Such reactions may be applied to conjugate radionuclides to GCC-targeted molecules described herein with an appropriate chelating agent and/or linker. See also Wensel and Meares (1983) Radioimmunoimaging and Radioimmunotherapy, Elsevier, N.Y., for techniques relating to the radiolabeling of polypeptides. See also, D. Colcher et al. Meth. Enzymol. 121: 802-816 (1986).

In embodiments, the compound or composition described herein is introduced into a patient in a detectable quantity and after sufficient time has passed for the compound to become associated with GCC-expressing cells, the compound is detected noninvasively. In another embodiment, the compound or composition is introduced into a patient, sufficient time is allowed for the compound to become associated with GCC-expressing cells, and then a sample of tissue from the patient is removed and the compound in the tissue is detected apart from the patient. In another embodiment, a biological sample is removed from a patient and a compound or composition described herein is introduced into the sample. After a sufficient amount of time for the compound to become bound to GCC-expressing cells, the compound is detected.

When a biological sample is removed from the patient, the compounds herein can be detected by immunohistochemistry on the sample, e.g., a biopsy sample. In such embodiments, the peptide and linker may be conjugated to a fluorophore, a dye, or another moiety detectable by visible light.

Exemplary biological samples for methods described herein comprise a cell, cells, tissue or body fluid, such as an inflammatory exudate, blood, serum, bowel fluid, stool sample. In particular embodiments, the biological sample comprises a cancerous cell(s) or tissue. For example, the sample can be a tumor biopsy, e.g., biopsy of a colorectal tumor, a gastric tumor, an esophageal tumor, a small intestine tumor, a lung tumor, a soft-tissue sarcoma, a neuroendocrine tumor, a neuroectodermal tumor, or from a tissue sample from any metastatic site thereof. In other embodiments, the biological sample can be blood or another fluid, where the fluid comprises a cancer cell.

The diagnostic methods described herein can comprise, e.g., responsive to the detected level of GCC, providing a diagnosis, a prognosis, an evaluation of the efficacy of treatment, or the staging of a disorder. A higher level of GCC in the sample or subject, as compared to the control material, indicates the presence of a disorder associated with increased expression of GCC. A higher level of GCC in the sample or subject, as compared to the control material, can also indicate the relative lack of efficacy of a treatment, a relatively poorer prognosis, or a later stage of disease. The level of GCC can also be used to evaluate or select future treatment, e.g., the need for more or less aggressive treatment, or the need to switch from one treatment regimen to another. In some embodiments, the methods further comprise selecting a GCC-targeted therapy, e.g., a GCC-targeted therapy described herein, based, at least in part, on the determined GCC levels, and optionally administering the selected GCC-targeted therapy to the subject.

In certain embodiments, the disclosure provides methods for determining the dose, e.g., radiation dose, that different tissues are exposed to when a subject, e.g., a human subject, is administered a GCC-targeted compound that is conjugated to a radioactive isotope. The method may include: (i) administering a GCC-targeted compound as described herein, e.g., that is labeled with a radioactive isotope, to a subject; (ii) measuring the amount of radioactive isotope located in different tissues, e.g., tumor, or blood, at various time points until some or all of the radioactive isotope has been eliminated from the body of the subject; and (iii) calculating the total dose of radiation received by each tissue analyzed. The measurements can be taken at scheduled time points, e.g., day 1, 2, 3, 5, 7, and 12, following administration (at day 0) of the radioactively labeled molecule to the subject. The concentration of radioisotope present in a given tissue, integrated over time, and multiplied by the specific activity of the radioisotope can be used to calculate the dose that a given tissue receives. Pharmacological information generated using GCC-targeted compounds labeled with one radioactive isotope, e.g., a gamma-emitter, e.g., ¹¹¹In can be used to calculate the expected dose that the same tissue would receive from a different radioactive isotope which cannot be easily measured, e.g., a beta-emitter, e.g., ⁹⁰Y. Additional details on measuring the expected dose are provided below in the Examples.

In some embodiments, the diagnostic method comprises a first detection step using a GCC-binding compound as described herein, e.g., a compound of Formula (VI) or (VIa), and a second detection step using a second GCC-binding compound. The two detection steps need not be performed on the same sample or at the same time. In some embodiments, performing two detection events can improve the reliability or reproducibility of the diagnosis by reducing the likelihood of a false positive or a false negative result. The second GCC-binding compound, in some embodiments, binds to a different region on GCC from the region that is bound by the first GCC-binding compound (e.g., a compound of Formula (VI) or (VIa)). The second GCC-binding compound may be, e.g., a rabbit antibody. The second GCC-binding compound may be, e.g. an anti-GCC molecule having CDRs listed in Table 3. The anti-GCC antibody molecule may comprise a heavy chain CDR1 of SEQ ID NO: 15, a heavy chain CDR2 of SEQ ID NO: 16, a heavy chain CDR3 of SEQ ID NO: 17, a light chain CDR1 of SEQ ID NO: 18, a light chain CDR2 of SEQ ID NO: 19, and a light chain CDR3 of SEQ ID NO: 20. The anti-GCC antibody molecule may be capable of competing for binding with, or be capable of binding to the same epitope as, an anti-GCC antibody comprising three heavy chain complementarity determining regions (CDR1, CDR2, and CDR3) comprising a heavy chain CDR1 of SEQ ID NO: 15, a heavy chain CDR2 of SEQ ID NO: 16, a heavy chain CDR3 of SEQ ID NO: 17, a light chain CDR1 of SEQ ID NO: 18, a light chain CDR2 of SEQ ID NO: 19, and a light chain CDR3 of SEQ ID NO: 20. The anti-GCC antibody molecule may comprise a heavy chain CDR1 of SEQ ID NO: 21, a heavy chain CDR2 of SEQ ID NO: 22, a heavy chain CDR3 of SEQ ID NO: 23, a light chain CDR1 of SEQ ID NO: 24, a light chain CDR2 of SEQ ID NO: 25, and a light chain CDR3 of SEQ ID NO: 26. The anti-GCC antibody molecule may be capable of competing for binding with, or be capable of binding to the same epitope as, an anti-GCC antibody comprising three heavy chain complementarity determining regions (CDR1, CDR2, and CDR3) comprising a heavy chain CDR1 of SEQ ID NO: 21, a heavy chain CDR2 of SEQ ID NO: 22, a heavy chain CDR3 of SEQ ID NO: 23, a light chain CDR1 of SEQ ID NO: 24, a light chain CDR2 of SEQ ID NO: 25, and a light chain CDR3 of SEQ ID NO: 26.

8.1 Positron Emission Tomography

PET signals are quantitative, have high specificity, and have high sensitivity.

PET imaging using the compounds and compositions described herein has technical benefits, e.g., over immunohistochemistry (IHC) based methods. For example, PET imaging is comprehensive allowing for simultaneous imaging of the whole body, whereas IHC is limited to one or a few biopsy samples. Thus, PET imaging can detect previously unknown lesions, while IHC is limited to lesions of which the physician was already aware. In addition, PET imaging does not require taking a biopsy sample and can image lesions that are inaccessible to biopsy. Further, PET imaging can be performed at one or more timepoints, whereas IHC can only assay a given biopsy sample at the timepoint the sample was collected. PET imaging also gathers data in real time, without a lag for processing biopsied samples. Thus, in some embodiments, PET imaging provides a good predictor of a patient's response to treatment with a GCC-targeted treatment. In some embodiments, PET imaging of a ⁶⁸Ga-labeled compound provides a greater signal to background ratio than other GCC-targeted diagnostics.

In some embodiments, two diagnostic methods are performed on the patient. For instance, the first diagnostic method is PET-detection of a compound described herein, and the second diagnostic method is IHC-based.

PET data can be quantified over multiple timepoints using time-activity curves. For example, a user may draw a region of interest (ROI) over a tissue such as a tumor, and generate time-activity curves for each ROI. The time-activity curve is plotted with activity (signal) on the y axis and time on the x-axis. This information can show whether different tumors or even different regions of a single tumor have the same or different properties.

### 9. Diseases to be Treated or Imaged with GCC-Targeted Compounds

The compounds and compositions described herein can be used to image, and/or as part of a treatment for, any disease in which GCC is expressed, and in which a doctor wants to deliver a therapeutic to the GCC expressing tissue. An example of this type of disease is cancer. GCC is expressed, e.g., in colorectal cancer, gastric cancer, pancreatic cancer, esophageal cancer, cancer of the gastroesophageal junction, small intestine cancer, lung cancer, soft tissue sarcomas such as leiomyosarcomas and rhabdomyosarcomas, gastrointestinal and bronchopulmonary neuroendocrine tumors, and neuroectodermal tumors, and metastases derived from GCC-expressing primary tumors. An example of the last category is a liver metastases derived from a primary colorectal tumor. Exemplary GCC expressing disorders are described in more detail below.

Colorectal cancer generally results from uncontrolled growth of the epithelial cells of the colon or rectum. It can be diagnosed by sigmoidoscopy or colonoscopy. The most frequent mutation promoting colon cancer is a mutation in the APC gene, though genes elsewhere in the Wnt pathway also lead to the disease. The most common cell type in colorectal cancer is adenocarcinoma, and lymphoma and squamous cell carcinoma are rarer. CRC can be treated by surgical resection, chemotherapy, and radiation. Common chemotherapeutic agents include capecitabine, fluorouracil, irinotecan, leucovorin, oxaliplatin and UFT. A preferred CRC treatment is an anti-GCC antibody, e.g., an immunoconjugate comprising an anti-GCC antibody, e.g., an immunoconjugate described herein.

Pancreatic cancer is a malignant neoplasm arising from pancreatic cells. An initial diagnosis is often made based on the patient's self-reported symptoms, and the diagnosis is confirmed using an endoscopic needle biopsy or surgical excision of the tissue. The most common cell type is the adenocarcinoma. Treatment options include surgery, radiation, and chemotherapy (for instance with gemcitabine, erlotinib, or oxiplatin). A preferred treatment is an anti-GCC antibody, e.g., an immunoconjugate comprising an anti-GCC antibody, e.g., an immunoconjugate described herein.

Esophageal cancer is malignancy of the esophagus. Biopsy is the primary method of diagnosis. The major cell types involved are squamous cells and adenocarcinoma. Treatment options include surgical resection, radiation, laser therapy, and chemotherapy (for example with cisplatin, carboplatin, or oxaliplatin, 5-FU, epirubicin, or capecitabine). A preferred treatment is an anti-GCC antibody, e.g., an immunoconjugate comprising an anti-GCC antibody, e.g., an immunoconjugate described herein.

There are two main forms of lung cancer, small cell lung cancer and non small-cell lung cancer. There are at least four distinct types of NSCLC, including adenocarcinoma, squamous cell, large cell, and bronchioalveolar carcinoma. Squamous cell (epidermoid) carcinoma of the lung is a microscopic type of cancer most frequently related to smoking. Adenocarcinoma of the lung accounts for over 50% of all lung cancer cases in the U.S. This cancer is more common in women and is still the most frequent type seen in non-smokers. Large cell carcinoma, especially those with neuroendocrine features, is commonly associated with spread of tumors to the brain. When NSCLC enters the blood stream, it can spread to distant sites such as the liver, bones, brain, and other places in the lung. Lung cancer is usually diagnosed by X-ray or CT scan, with a biopsy for confirmation. Lung cancer can be treated with surgery, radiation, or chemotherapy (for instance with itabine, carboplatin, gemcitabine, paclitaxel, vinorelbine, topotecan, irinotecan, docetaxel, pemetrexed, or etoposide). A preferred treatment is an anti-GCC antibody, e.g., an immunoconjugate comprising an anti-GCC antibody, e.g., an immunoconjugate described herein.

### 10. Companion Diagnostic for GCC-Targeted Therapy

Because the compounds and compositions described herein distinguish between GCC-expressing tumors and non-GCC-expressing tumors, the compounds can identify patients likely to respond to a GCC-targeted therapy, e.g., an anti-GCC antibody, e.g., an immunoconjugate comprising an anti-GCC antibody, e.g., MLN0264.

The in vitro and in vivo diagnostic methods described herein are useful to inform whether a patient suffering from a proliferative disease such as cancer, or a gastrointestinal disorder such as inflammatory bowel syndrome, Crohn's Disease or constipation, or should be treated or not with a GCC-targeted therapy, based on the presence or absence, respectively, of GCC expression on the surface of or within the patient's cells or tissue. A patient having one more cells that express GCC on the cell surface or within the cell is a candidate for treatment with a GCC-targeted therapy.

Exemplary diseases/disorders that may be evaluated (e.g., diagnosed) and treated using the companion diagnostic methods described herein include, but are not limited to, proliferative disorders including but not limited to colorectal cancer, stomach cancer, small intestine cancer, esophageal cancer, cancer of the gastroesophageal junction, pancreatic cancer, lung cancer (e.g., squamous cell carcinoma, adenosquamous carcinoma, adenocarcinoma), soft tissue sarcoma such as leiomyosarcoma and rhabdomyosarcoma, gastrointestinal and bronchopulmonary neuroendocrine tumors, and neuroectodermal tumors.

The methods of the invention guide physician's decisions in determining whether to treat a patient with a GCC-targeted therapy. The methods provided herein also allow for the generation of a personalized treatment report, e.g., a personalized cancer treatment report, e.g., with a GCC-targeted therapy described herein.

In one embodiment, GCC-targeted therapy is an immunoconjugate comprising an anti-GCC antibody conjugated to the potent microtubule inhibitor monomethyl auristatin E (MMAE) via a protease cleavable linker. In one embodiment, the immunoconjugate has the following formula I-5:

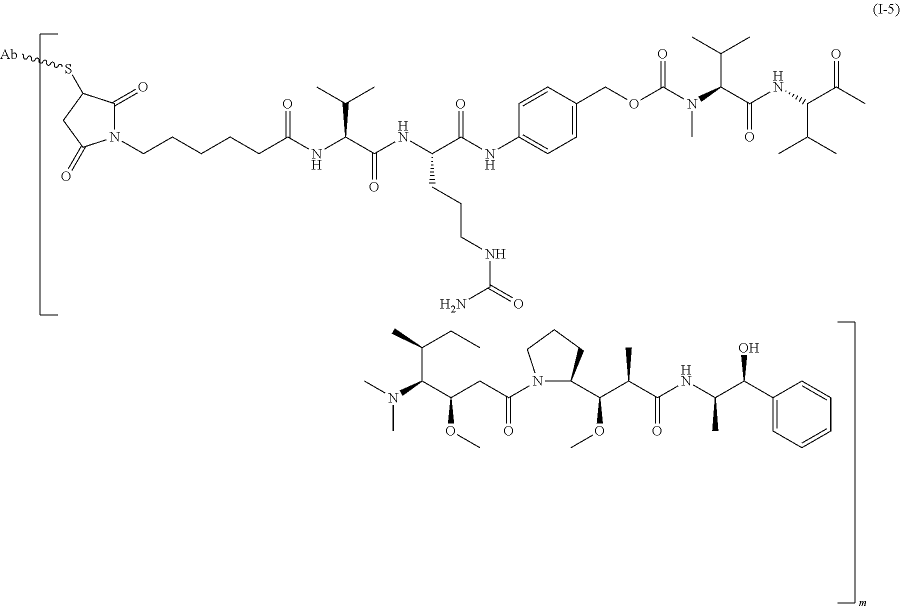

or a pharmaceutically acceptable salt thereof, wherein:
Ab is an anti-GCC antibody molecule, or antigen binding fragment thereof, and

m is an integer from 1-8 (e.g., 3-5, 4).

In certain embodiments, the immunoconjugate of Formula (I-5) comprises an anti-GCC antibody molecule, which includes: three light chain CDRS from Table 3, and three heavy chain CDRs from Table 3. In embodiments, the anti-GCC antibody molecule is anti-GCC human IgG1 monoclonal antibody that includes a light chain variable region (VL) having the three light chain complementarity determining regions (CDR1, CDR2, and CDR3) and a heavy chain variable region (VH) having the three heavy chain complementarity determining regions (CDR1, CDR2, and CDR3) listed in Table 3 below. In embodiments the CDRs are those indicated by SEQ ID NOS: 5-10. In embodiments the CDRs are those indicated by SEQ ID NOS: 15-20. In embodiments the CDRs are those indicated by SEQ ID NOS: 21-26. In one embodiment, the anti-GCC antibody molecule includes a heavy chain variable region and light chain variable region listed in Table 4 below.

TABLE 3
Amino acid sequence of VL CDRs and VH CDRs

5F9

| | | |
|---|---|---|
| VH | SEQ ID | GYYWS |
| CDR1 | NO: 5 | |
| VH | SEQ ID | EINHRGNTNDNPSLKS |
| CDR2 | NO: 6 | |
| VH | SEQ ID | ERGYTYGNFDH |
| CDR3 | NO: 7 | |
| VL | SEQ ID | RASQSVSRNLA |
| CDR1 | NO: 8 | |
| VL | SEQ ID | GASTRAT |
| CDR2 | NO: 9 | |
| VL | SEQ ID | QQYKTWPRT |
| CDR3 | NO: 10 | |

MIL-44-148-2-H2 and MIL-44-148-2-L5

| | | |
|---|---|---|
| VH | SEQ ID | SHRMN |
| CDR1 | NO: 15 | |
| VH | SEQ ID | IITHNSITYYASWAKS |
| CDR2 | NO: 16 | |
| VH | SEQ ID | EDSMGYYFDL |
| CDR3 | NO: 17 | |
| VK | SEQ ID | QASQSISNWLA |
| CDR1 | NO: 18 | |
| VK | SEQ ID | RASTLAS |
| CDR2 | NO: 19 | |
| VK | SEQ ID | QQTYTNNHLDNG |
| CDR3 | NO: 20 | |

MIL-44-67-4 H2 and MIL-44-67-4 L4

| | | |
|---|---|---|
| VH | SEQ ID | NYAIS |
| CDR1 | NO: 21 | |
| VH | SEQ ID | YISYGKSIYYASWAKG |
| CDR2 | NO: 22 | |
| VH | SEQ ID | EDSATYSPNL |
| CDR3 | NO: 23 | |
| VK | SEQ ID | QASQSINTYLA |
| CDR1 | NO: 24 | |
| VK | SEQ ID | RASTLAS |
| CDR2 | NO: 25 | |
| VK | SEQ ID | QQGYSYNNLDRA |
| CDR3 | NO: 26 | |

Briefly, rabbit monoclonal antibodies MIL-44-148-2 and MIL-44-67-4 were generated by traditional immunization technology in rabbits. True rabbit-rabbit hybridomas were generated at Epitomics (Burlingame, Calif.) by fusing isolated B-cells from an immunized rabbit with Epitomics' proprietary fusion partner cell line (see U.S. Pat. Nos. 7,402,409; 7,429,487; 7,462,697; 7,575,896; 7,732,168; and 8,062,867). Specificity of the antibodies against GCC was tested by ELISA and flow cytometry (FCM). Antibodies MIL-44-148-2 and MIL-44-67-4 are described in more detail in PCT/US2013/038542, the contents of which are herein incorporated by reference in their entirety.

TABLE 4
Amino acid sequence of antibody variable regions
of antibody 5F9

| | | |
|---|---|---|
| Heavy | SEQ ID | QVQLQQWGAGLLKPSETLSLTCAVFGGSFSGYYW |
| chain | NO: 11 | SWIRQPPGKGLEWIGEINHRGNTNDNPSLKSRVT |
| | | ISVDTSKNQFALKLSSVTAADTAVYYCARERGYT |
| | | YGNFDHWGQGTLVTVSS |
| Light | SEQ ID | EIVMTQSPATLSVSPGERATLSCRASQSVSRNLA |
| chain | NO: 12 | WYQQKPGQAPRLLIYGASTRATGIPARFSGSGSG |
| | | TEFTLTIGSLQSEDFAVYYCQQYKTWPRTFGQGT |
| | | NVEIK |

In embodiments, the anti-GCC antibody molecule comprises a heavy chain that comprises an amino acid sequence of SEQ ID NO: 11, a light chain that comprises an amino acid sequence of SEQ ID NO: 12, or both.

In embodiments, the anti-GCC antibody molecule includes a heavy chain constant regions and a light chain constant region, e.g., having an amino acid sequence provided below.

A heavy chain constant region amino acid sequence:

(SEQ ID NO: 13)
MGWSCIILFLVATATGVHSQVQLQQWGAGLLKPSETLSLTCAVFGGSFSGY
YWSWIRQPPGKGLEWIGEINHRGNTNDNPSLKSRVTISVDTSKNQFALKLS
SVTAADTAVYYCARERGYTYGNFDHWGQGTLVTVSSASTKGPSVFPLAPSS
KSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQ
KSLSLSPGK

A kappa light chain constant region amino acid sequence:

(SEQ ID NO: 14)
MGWSCIILFLVATATGVHSEIVMTQSPATLSVSPGERATLSCRASQSVSRN
LAWYQQKPGQAPRLLIYGASTRATGIPARFSGSGSGTEFTLTIGSLQSEDF
AVYYCQQYKTWPRTFGQGTNVEIKRTVAAPSVFIFPPSDEQLKSGTASVVC
LLNNFYPREAKVQWKVDNALQSGNSQESVTEQDSKDSTYSLSSTLTLSKAD
YEKHKVYACEVTHQGLSSPVTKSFNRGEC

An exemplary GCC-targeted therapy is MLN0264 characterized by Formula I-5. MLN0264 is an antibody-drug conjugate useful for treating cancer. The antibody is a fully human monoclonal antibody that recognizes GCC. Its variable regions and CDRs are provided above in Tables 3 and 4. The drug is monomethylauristatin E (MMAE), a small molecule microtubule inhibitor that results in cell cycle arrest and apoptosis.

A more detailed description of such GCC-targeted therapeutics, can be found in International Application WO2013/163633, the contents of which is incorporated herein by reference.

### 11. Methods of Treatment

A GCC-targeted therapeutic can be administered at a dose and frequency selected by the treating physician. The GCC-targeted therapeutic can be used to treat any disease that is characterized by unwanted cells expressing GCC. Examples of these diseases are colorectal cancer, gastric cancer, pancreatic cancer, esophageal cancer, lung cancer, and metastases derived from GCC-expressing primary tumors.

In some embodiments, the compounds or compositions described herein can be used to deliver a therapeutic radionuclide to tumor cells. In some embodiments, the radionuclide is an alpha-emitter. Alpha particles travel only for short distances before becoming absorbed, so alpha-emitters will deliver a localized dose of radiation to the tumor without irradiating healthy tissues.

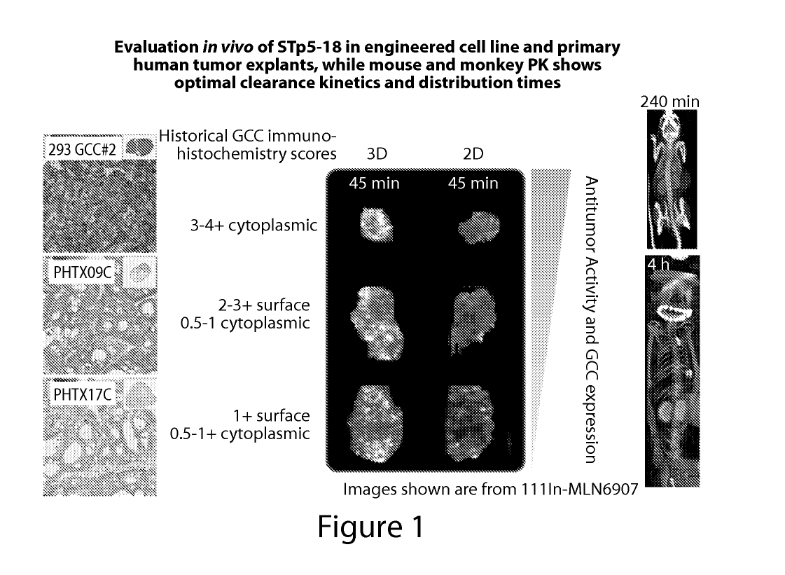

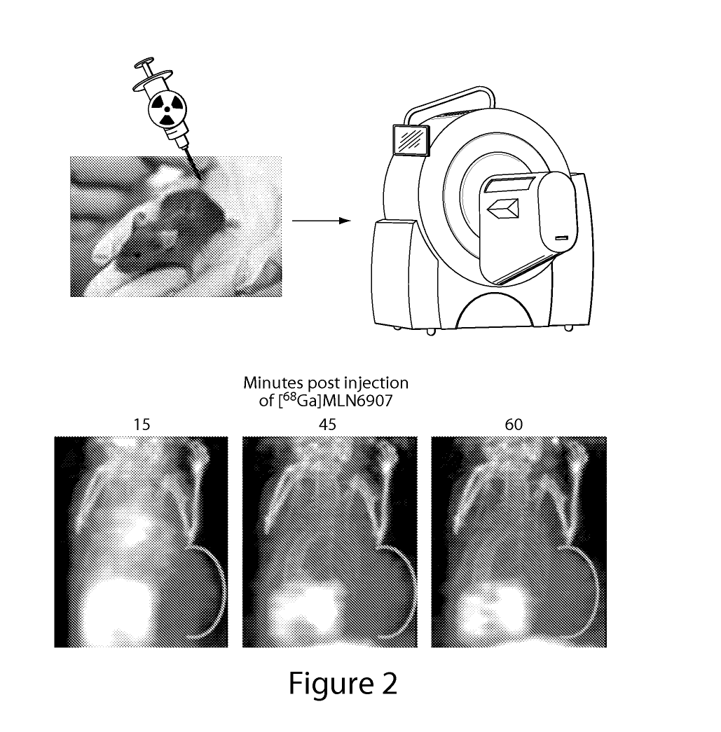

Targeted Alpha Particle Therapy (TAT) is an emerging approach in cancer therapy. Alpha-particles have a high linear energy transfer rate (4-9 MeV) causing high cellular toxicity, within a short range (<0.1 mm) in tissue which helps to mitigate side effects in normal tissues. The high energy transfer results in double strand DNA breaks and lethality. 225^{Ac} has a 10-day half-life and generates 4 alpha emissions with a total energy of 27 MeV, after rapid sequential daughter decay to its stable form, ²⁰⁹Bi. However, after decay, the daughter isotopes can be released from a DOTA chelator either due to the recoil energy or the different chemical properties of the first daughter nuclide ²²¹Fr. The a-emitting daughter nuclides ²²¹Fr (t_{1/2}=4.8 m), ²¹⁷At (t_{1/2}=32 ms) and ²¹³Bi (t_{1/2}=45.6 min), have very short half-lives but are still able to distribute within the body and accumulate in normal tissue. An example is ²¹³Bi which is known to traffic to the kidneys. See McDevitt M R et al. (2002), Cell Death & Differentiation, including FIG. 1. Exemplary alpha emitters suitable for therapeutic use include ²¹³Bi and ²²⁵Ac.

In some embodiments, the radionuclide is a beta-emitter such as ⁹⁰Y or ¹⁷⁷Lu. Beta emitters often have a total energy of 0.1-1 MeV, and can generally travel in the 1-10 mm range when administered to a subject.

In other embodiments, the compounds or compositions described herein can be used to deliver a detectable radionuclide to the tumor cells, and then e.g., a GCC-targeted therapeutic, e.g., a GCC-targeted therapeutic described herein, can be administered to a subject that has a detected GCC-expressing tumor.

In some embodiments, the patient is a metastatic patient who has received at least one, two, three, or more standard of care therapies for the cancer. In other embodiments, the patient has received no other standard of care therapies for the cancer. In some embodiments, the patient does not tolerate certain standard of care therapies like oxaliplatin, irinotecan, or fluoropyrimidine. In preferred embodiments, the patient has one or more cancer cells expressing GCC.

In one embodiment, the invention provides a method of treating cancer by administering an anti-GCC antibody molecule or an immunoconjugate comprising an anti-GCC antibody molecule (e.g., described herein) to a patient in need of such treatment. The method can be used for the treatment of any cancerous disorder which includes at least some cells that express the GCC antigen, e.g., as determined using a compound or composition described herein. As used herein, the term “cancer” is meant to include all types of cancerous growths or oncogenic processes, metastatic tissues or malignantly transformed cells, tissues, or organs, irrespective of histopathologic type or stage of invasiveness. The terms “cancer” and “tumor” may be used interchangeably (e.g., when used in the context of treatment methods, “treatment of a cancer” and “treatment of a tumor” have the same meaning).

In embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting colon. Adenocarcinomas include malignancies such as non-small cell carcinoma of the lung. Metastatic lesions of the aforementioned cancers can also be treated or prevented using the methods and compositions of the invention.

In some embodiments, the GCC-expressing cancer to be treated is a primary or metastatic cancer of gastrointestinal origin, such as colorectal cancer, stomach cancer, small intestine cancer, or esophageal cancer. In some embodiments, the GCC-expressing cancer to be treated is primary or metastatic pancreatic cancer. In some embodiments, the GCC-expressing cancer to be treated is primary or metastatic lung cancer, such as squamous cell carcinoma, adenosquamous carcinoma, or adenocarcinoma. In some embodiments, the GCC-expressing cancer to be treated is a sarcoma, such as leiomyosarcoma or rhabdomyosarcoma. In some embodiments, the GCC-expressing cancer to be treated is a primary or metastasized neuroectodermal tumor, such as aphaechromotcytoma or a paraganglioma. In some embodiments, the GCC-expressing cancer is a primary or a metastasized bronchopulmonary or a gastrointestinal neuroendocrine tumor.

The method can be useful in treating a relevant disorder at any stage or subclassification. For example, method can be used to treat early or late stage colon cancer, or colon cancer of any of stages 0, I, IIA, IIB, IIIA, IIIB, IIIC, and IV.

### 12. Combination Therapies

The methods of treatment described herein can include the use of a GCC-targeted therapy, e.g., a GCC targeted therapy described herein, e.g., MLN0264, in combination with other therapies. For example, the combination therapy can include a composition comprising a GCC-targeted therapy described herein co-formulated with, and/or co-administered with, one or more additional therapeutic agents, e.g., one or more anti-cancer agents, e.g., cytotoxic or cytostatic agents, hormone treatment, vaccines, and/or other immunotherapies. In other embodiments, the GCC-targeted therapy is administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the GCC-targeted therapy, e.g., MLN0264, is used in combination with a chemotherapeutic agent, e.g., a DNA damaging chemotherapeutic agent. Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

In some embodiments, the radiolabeled peptide and/or the radiolabel has high uptake by the kidney. A possible approach to reduce kidney uptake is the coadministration of a protectant which changes the charge of renal tubules and decrease the absorption of peptides and free radiolabel, e.g. ²¹³Bi. In one embodiment, coadministration comprises administering the radiolabeled peptide prior to, concurrently with and/or after administration with the protectant. By using a kidney protectant, the maximum tolerance dose (MTD) could be increased and nephropathy reduced. In some embodiments, the kidney protectant is selected from Clinisol, lysine, lysine/arginine, gelofusine and amifostine, or any combination thereof. In embodiments, Clinisol comprises: lysine, leucine, phenylalanine, valine, histidine, isoleucine, methionine, threonine, tryptophan, alanine, arginine, glycine, proline, glutamic acid, serine, aspartic acid, and tyrosine, and has an osmolarity of about 1357 mOsmol/L. In embodiments, lysine/arginine comprises substantially equal proportions of lysine and arginine. In embodiments, Gelofusine is a solution of succinylated gelatin, e.g., a 4% w/v solution and can be used as an intravenous colloid. In embodiments, Amifostine is 2-(3-aminopropylamino)ethylsulfanyl phosphonic acid.

In some embodiments, the GCC-targeting compound or composition is administered in combination with a treatment that increases urinary excretion. In one embodiment, the agent which increases urinary excretion, is administered prior to, concurrently with and/or after administration with the radiolabeled peptide. In one embodiment, the agent which ameliorates bladder toxicity associated with therapy is saline, e.g., intravenous saline, D5 half normal saline or D5 water.

### EXAMPLES

### Example 1

### Producing a Soluble Formulation for Radiolabeling

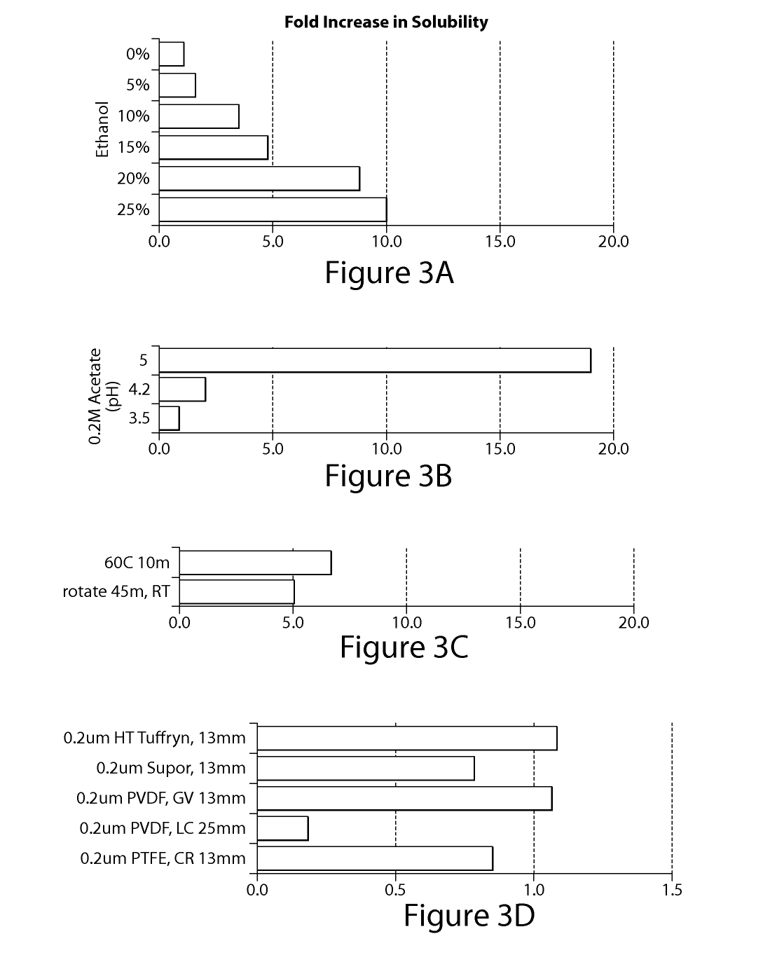

Bulk DSP1 has a low solubility of about 0.1 mg/ml, and an aqueous solution formulation was developed. This aqueous formulation is referred to as DSP2. Solubility could be increased in several ways. First, the addition of ethanol (for instance in the range of 5-25%) increases solubility up to 10-fold. This can be seen in FIG. 3A, where increasing the ethanol concentration increases solubility of DSP1. Ethanol can also enhance labeling efficiency. Second, increasing the pH of the solvent from 3.5 to 5 increased solubility by about 19-fold (to above 1 mg/ml), as seen in FIG. 3B. However, this advantage in solubility is offset by the decrease in labeling efficiency at higher pHs, as shown in the following section. Third, heat and mechanical mixing improved solubility. In particular, FIG. 3C shows that heating for 60° for 10 minutes, or shaking for 45 minutes at room temperature, increases solubility by about 5 to 7-fold. FIG. 3D shows retention of the compound on various filters.

DSP1 can be converted to soluble DSP2 according to the following protocol. The frozen MLN6907 DSP1 is allowed to equilibrate at controlled room temperature (RT). The required amount of DSP1 is weighed and dissolved in 0.02M solution of sodium acetate. To this solution 1% sucrose, 10% ethanol and 0.18M glacial acetic acid is sequentially added. Sufficient water (low metal content) is then added and mixed until a homogenous solution is formed. Samples are taken for in-process testing which includes pH, density, concentration by UV and bioburden testing before sterile filtration.

The formulated solution is then subjected to sterile filtration. Using two 0.2 μm PVDF sterilizing filters in series, the formulated solution of MLN6907 DSP1 is filtered in a sterile bag. The bag is stored at 2-8° C. until aseptic filling. The filters are tested for integrity before and after use. The filtered solution is then dispensed into vials, with 2 mL per vial.

### Example 2

### Providing Gallium-68

In the generator, Gallium-68 is continuously produced through the decay of Germanium-68 by electron capture and is eluted off a 68Ge/68Ga generator with dilute hydrochloric acid (HCl). In brief, a syringe is used to push a dilute HCl solution into the inlet line connected to the generator column. The 68Ga present on the column, which has been produced by the decay of 68Ge during the time since the last elution, becomes dissolved in the HCl, and is eluted off.

### Example 3

### Labeling the Compound

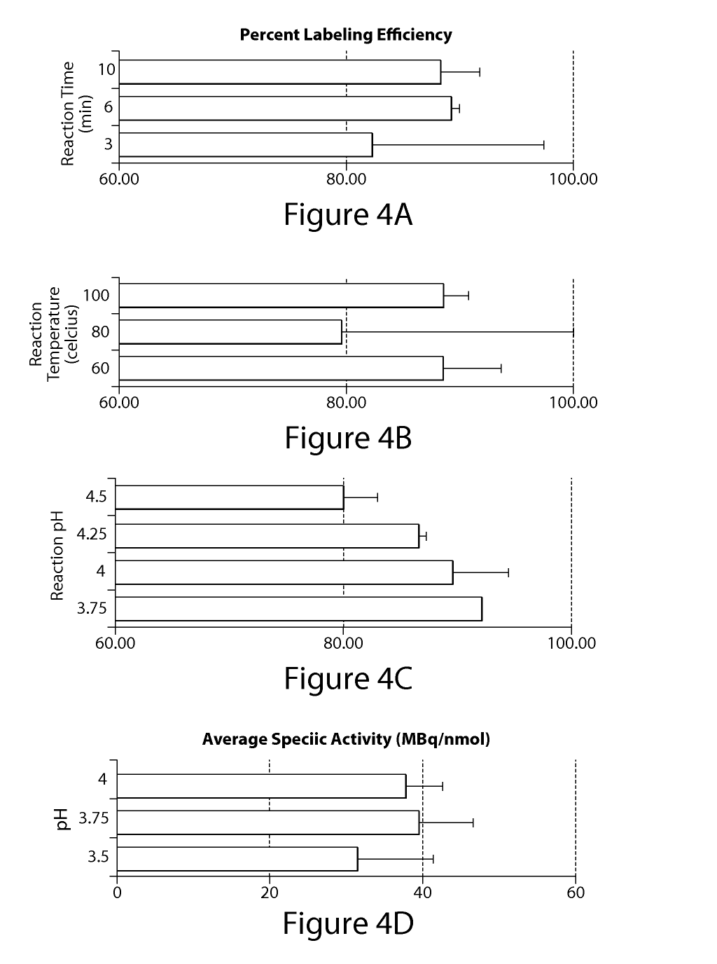

Various parameters were tested to identify suitable conditions for labeling the DSP2 compound with Gallium-68. First, as shown in FIG. 4A, the highest yields were achieved with a reaction time of 6-10 minutes. Second, as shown in FIG. 4B, a reaction temperature of 100° C. achieved the lowest variability in labeling levels. Optimization of pH levels is shown in FIG. 4C. A pH range between 3.75 and 4.0 results in high labeling levels. FIG. 4D shows suitable pH levels for the reaction. The reaction pH was determined based on the labeling efficiency. A suitable buffer was 0.2M sodium acetate, which resulted in a solution pH of 3.8. Unless otherwise specified, the reactions in the FIG. 4A-D experiments were carried out at 100° C. and pH 4 for 10 minutes.

Generally, the unlabeled DSP is provided in stoichiometric excess to the radiolabel, to minimize the amount of free radiolabel. This practice should minimize background attributable to free radiolabel during image collection.

In a clinical setting, the [⁶⁸Ga]MLN6907 DS is manufactured in a semi-closed automated system (Eckert & Ziegler Modular-Lab PharmTracer) utilizing single-use, sterile, disposable cassettes. The cassettes are assembled under GMP-compliant clean room conditions, sterilized with gamma radiation and double vacuum-packed. The [68Ga]MLN6907 DS is sterile filtered (0.2 μm filter) into a sterile vial to generate the drug product.

It was observed that the compound MLN6907 achieved a higher labeling efficiency than a related compound having the same peptide and chelating group as MLN6907, but a different linker.

### Example 4

### Purifying the Radiolabeled Compound

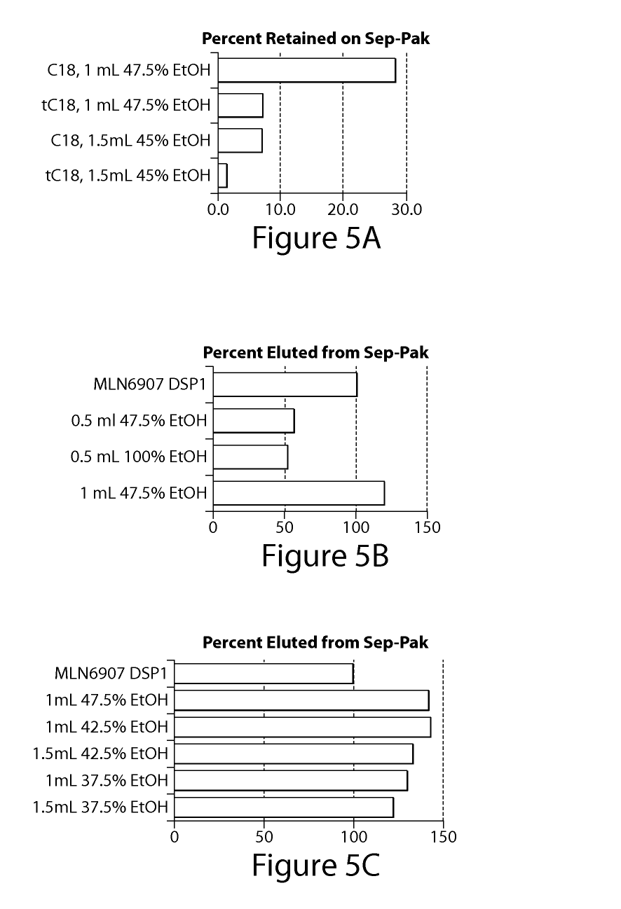

A Sep-Pak column was used to purify the radiolabeled compound. An average of 28% of the labeled material was retained on the Sep-Pak column after elution with 47.5% EtOH and saline wash. To improve purification efficiency, different columns were tested and the elution volume was increased. Specifically, FIG. 5A shows the percent of the radiolabeled compound retained on various columns. Using a tC18 column improved the amount of radiolabeled compound that could be recovered from the column. FIG. 5A also shows that increasing the elution volume of ethanol from 1 mL of 47.5% ethanol to 1.5 mL of 45% ethanol increased the amount of radiolabeled compound that could be recovered from the column. FIG. 5B shows that increasing the elution volume from 0.5 mL to 1 mL increases the percentage of compound that can be eluted from the column. FIG. 5C shows the amount of compound that can be eluted from the column using different columns and concentrations of ethanol.

### Example 5

### Characterizing GCC-Status of Tumors

GCC-targeted imaging and therapeutic agents will only be effective if cell types of interest express GCC. Therefore, the GCC-expressing status of six types of cancer was determined using Transcription-Mediated Amplification (TMA). TMA is a transcription-based amplification system in which reverse transcriptase is used to produce dsDNA from an RNA template, and RNA polymerase is used to make more RNA.

TABLE 5
GCC status of cancer cells

| | | % GCC Positive |
|---|---|---|
| Tumor Type | N Tested | Staining* |
| Colorectal | 298 | 95 |
| Gastric | 154 | 79 |
| Pancreatic | 221 | 63 |
| Esophageal | 138 | 30 |
| Lung Adenocarcinoma | 81 | 44 |
| Lung Squamous | 74 | 10.8 |

*% positive defined by H score >10

### Example 6

### In Vitro Pharmacology

In an in vitro competitive binding assay to evaluate the effects of the ⁶⁸GaCl₃ chelation reaction labeling conditions (mixing with acetone: HCl [98:2] and heating at approximately 100° C. for 10 minutes) on [⁶⁸Ga]MLN6907 DP (drug product) GCC affinity. [111In]MLN6907 was used as a surrogate for [⁶⁸Ga]MLN6907 DP because of the long incubation times necessary to perform assays (¹¹¹In half-life=2.8 days, ⁶⁸Ga half-life=68 minutes). The competition for binding to the ligand (GCC) and concomitant percentage displacement of radiolabeled [¹¹¹In]MLN6907 after the addition of increasing concentrations (8.5×10⁻⁶ to 500 nM) of cold peptide (untreated or treated MLN6907 DSP) was calculated by measuring the final radioactivity in the cells using a gamma counter. Treated and untreated (with and without chelation reaction conditions, respectively) MLN6907 DSP inhibited the binding of Indium-111 [¹¹¹In]MLN6907 with concentration producing 50% inhibition (IC50) of 4.75 and 4.59 nM, respectively, in human embryonic kidney (HEK)-293 GCC Clone 2 cells (GCC-expressing HEK-293 cells). The difference in affinity between untreated and treated MLN6907 DSP was not significant (p>0.05). These results demonstrate that exposure of the DSP to the manufacturing process (heat and acetone) did not impact binding affinity of treated MLN6907 DSP (mock-radiolabeled MLN6907 DP) to GCC under the current labeling conditions.

### Example 7

### In Vivo Pharmacology

Female CB-17 severe combined immunodeficient (SCID) mice bearing cell line or primary tumor derived xenografts were used to assess competitive binding and biodistribution of [⁶⁸Ga]MLN6907 DP in vivo. Four xenograft models that express different levels of GCC were used. Two models were derived from cell lines: HEK-293 GCC Clone 2 (human embryonic kidney) and T84 (colon carcinoma), and two models were primary colon adenocarcinoma human tumor models: PHTX-21C and PHTX-17C. In the mice, tumor cells or fragments and tumors were allowed to grow until reaching a volume of 200 to 700 mm³. GCC expression level, previously determined by IHC using a scoring system of 0-300 (H-score [apical and cytoplasmic]) to indicate GCC concentration in a separate set of tumor samples, as 300 and 270, 0 and 300, and 40 and 50 for T84, HEK-293 GCC Clone 2, and PHTX-17C, respectively. Mice were then administered a single IV dose of [68Ga]MLN6907 DP (0.01, 0.02, 0.15, or 0.2 μg) with increasing concentrations of MLN6907 DSP (0, 1.8, 1.85, or 20 μg). Tumor, blood, liver, and kidneys were collected at 1 hour postdose and assayed for ⁶⁸Ga activity using a gamma counter; uptake and biodistribution of [⁶⁸Ga]MLN6907 DP was determined as the % ID/g. The ratio of tumor to each tissue was determined to demonstrate the signal-to-background of [68Ga]MLN6907 DP.

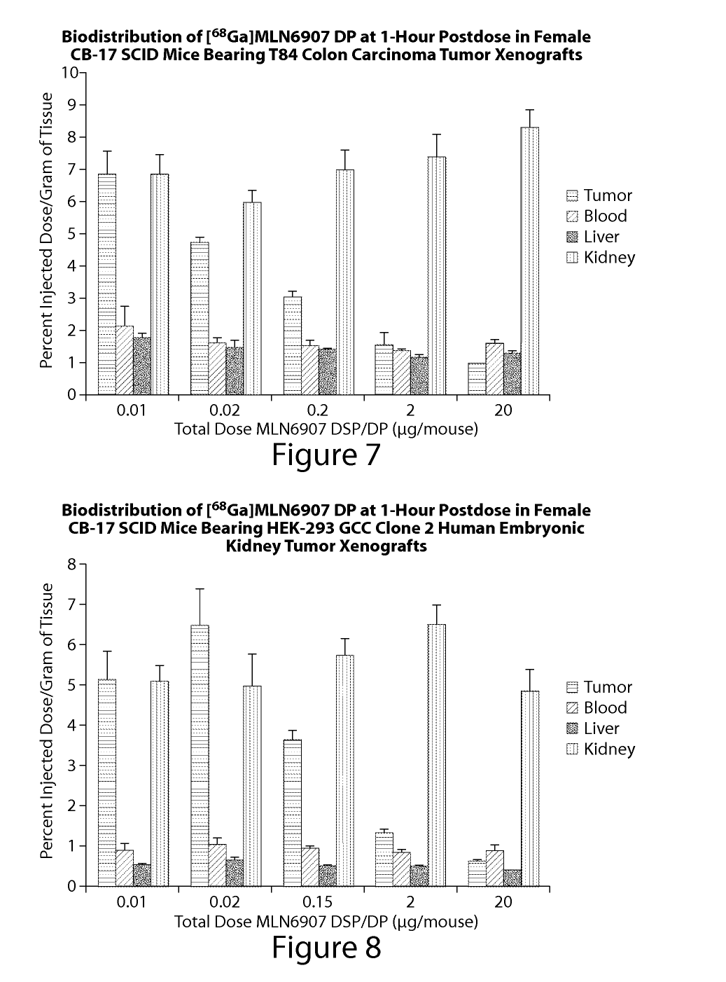

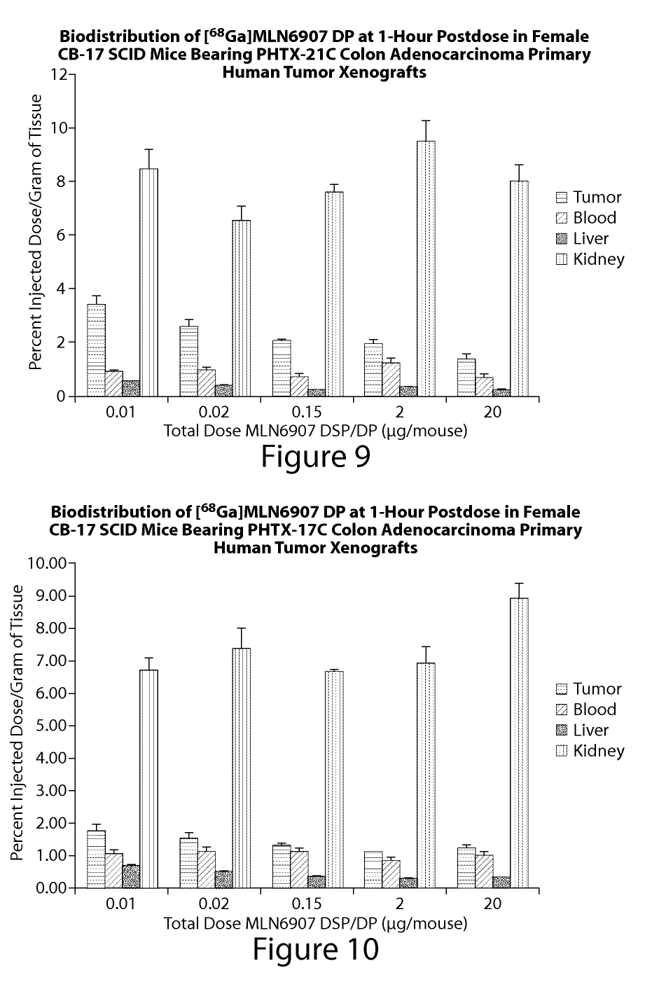

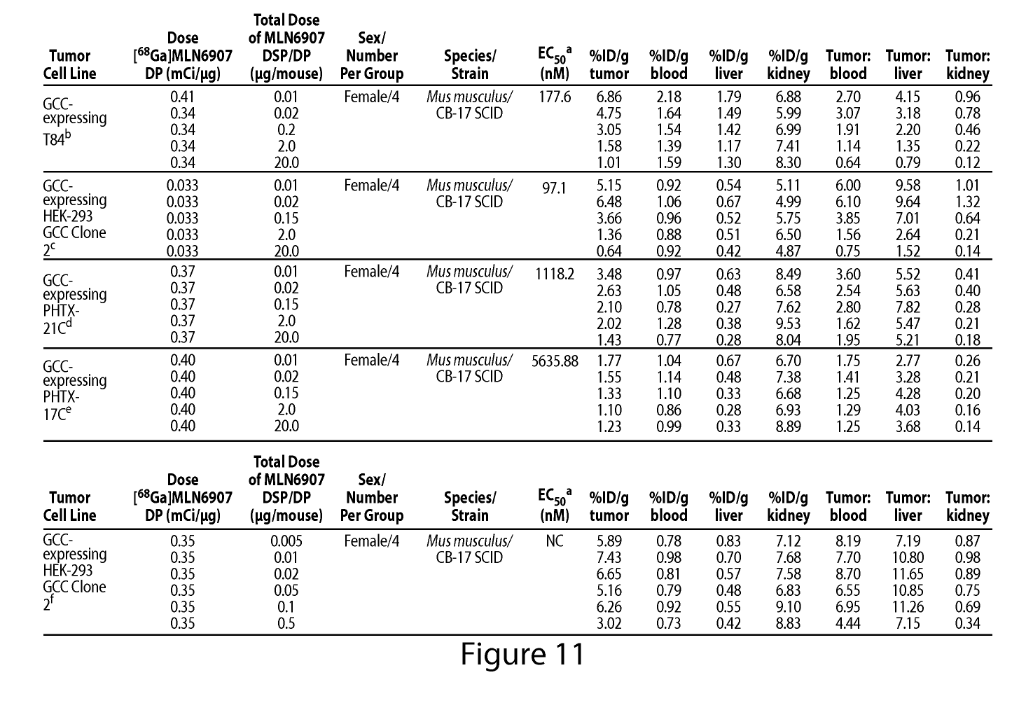

These competitive binding studies demonstrated that decreasing [⁶⁸Ga]MLN6907 DP specific activity (SA) with the addition of increasing concentrations of MLN6907 DSP resulted in a reduction in tumor uptake of [⁶⁸Ga]MLN6907 DP (percent injected dose [% ID] per gram of tissue [% ID/g]) in mice in all models tested FIGS. 7, 8, 9, 10, and 11). The addition of MLN6907 DSP had no major effect on [⁶⁸Ga]MLN6907 DP uptake in non-GCC-expressing tissues (blood, liver, and kidney). Consequently, increasing concentrations of MLN6907 DSP at 1.8, 1.85 or 20 μg were able to displace the binding of [⁶⁸Ga]MLN6907 DP specifically in GCC-expressing tumor tissues, resulting in a measurable reduction in [68Ga]MLN6907 DP uptake (as measured by % ID/g) indicating specific GCC binding in vivo. The concentration producing half-maximal response (EC₅₀) for the in vivo competitive binding assays in these in vivo studies was 97.1 to 5659.5 nM in the tumor models with different GCC expression levels (as determined previously by immunohistochemistry). The in vivo EC₅₀ calculation is based on a semi-mechanistic model and is affected by peptide extravasation, binding affinity, and internalization rate. A simple pharmacokinetic (PK)/pharmacodynamic model projected an optimal [⁶⁸Ga]MLN6907 DP mass dose <100 μg for clinical imaging studies.

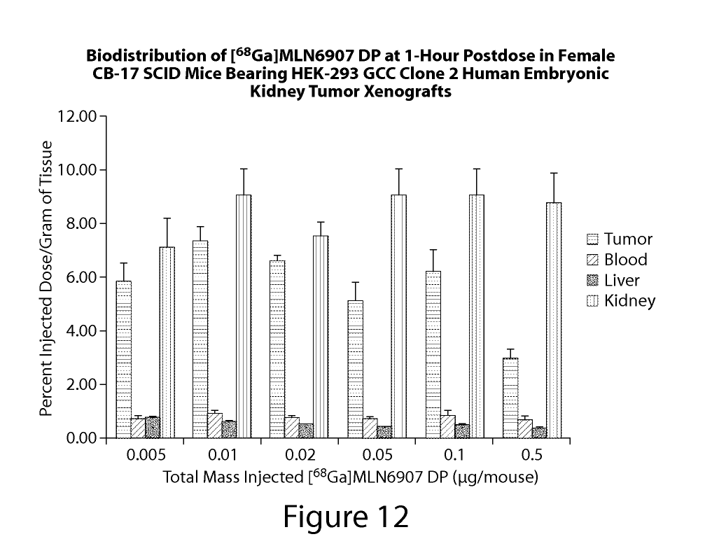

An additional in vivo study was conducted using HEK-293 GCC clone 2 human embryonic kidney tumor xenografts in female CB-17 SCID mice to evaluate the effect of increasing the mass dose of [⁶⁸Ga]MLN6907 DP on tumor uptake. [68Ga]MLN6907 DP was administered to female CB-17 SCID mice bearing HEK-293 GCC Clone 2 HEK tumor xenografts at a mass of 0.005 to 0.50 μg (FIG. 12). Administration of [68Ga]MLN6907 DP at 0.01 μg (SA=0.091 mCi/m) demonstrated maximal uptake of 7.43% ID/g in the tumor compared with 0.98% ID/g, 0.70% ID/g, and 7.68% ID/g in the blood, liver, and kidney, respectively, resulting in tumor-to-tissue ratios of 7.70, 10.80, and 0.98, respectively. Increased [⁶⁸Ga]MLN6907 DP mass resulted in reduced uptake in the tumor at the high mass dose (0.50 μg), indicating that there is competitive binding with high mass injection even when SA is maintained. These results, along with those from the competitive binding studies, demonstrate that high SA of [⁶⁸Ga]MLN6907 DP, which reduced the concentration of unlabeled peptide that can compete with the ⁶⁸Ga-labeled peptide in the final drug product, is more effective than increased mass to facilitate a robust PET image of tumor uptake.

### Example 8

### Pharmacokinetic Modeling Studies

Tumor kinetics and clearance of a GCC-binding compound, such as [68Ga]MLN6907, can be modeled using a mechanistic model. This modeling was performed with [68Ga]MLN6907. The modeling centers on nine parameters which are set out in Table 6 below.

TABLE 6

| Parameters | Description | Results | |
|---|---|---|---|
| MW | Molecular Weight | 2174.6 | daltons |
| P_{cell} | Cell density | 3 × 10⁸ | cells/ml |
| T_{1/2, kresid} | Residualization rate | >24 | hours |
| K_{D} | Affinity | 3.2 | nM |
| T_{1/2, ke} | Half-life of internalization | 56 | minutes |
| PK | In vivo blood PK | 26 | minutes |
| Bₘₐₓ | Antigen surface density | ~14,000 (in vivo) | |
| | (HEK-293 GCC Clone 2) | (~20,000 in vitro) | |
| R | Vascularity | 20-70 | um |
| | (capillary half-distance) | | |
| SA | Radioactivity per unit mass | 26-31 | MBq/nmole |

Some parameters were known at the outset of the study, and others were determined experimentally. In these experiments, Indium-111 was used as a surrogate for Gallium-68 because the longer half-life of Indium was helpful in conducting some longer-duration assays.

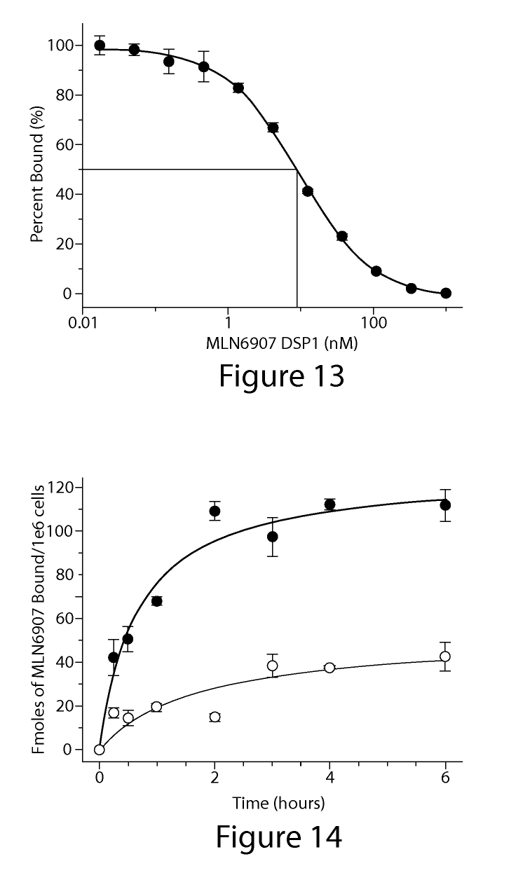

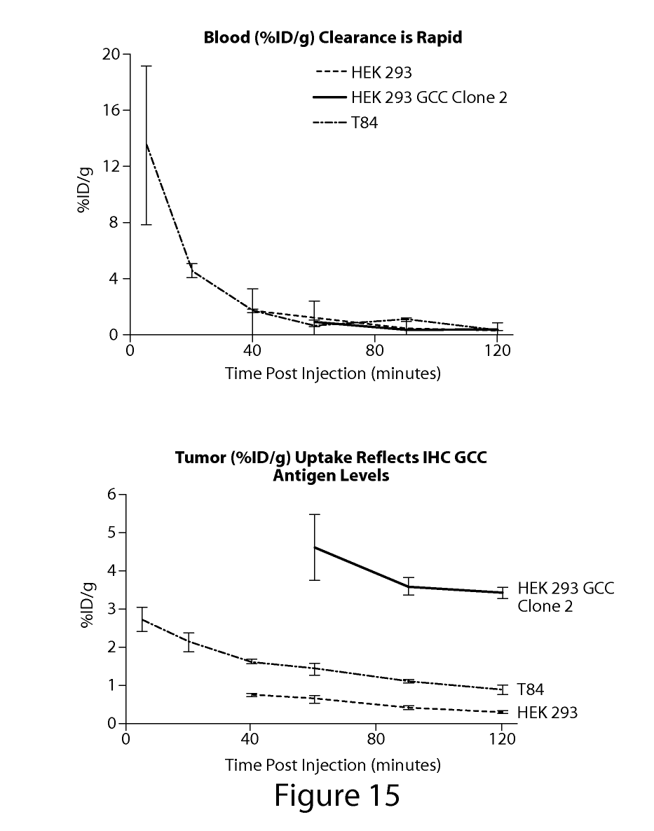

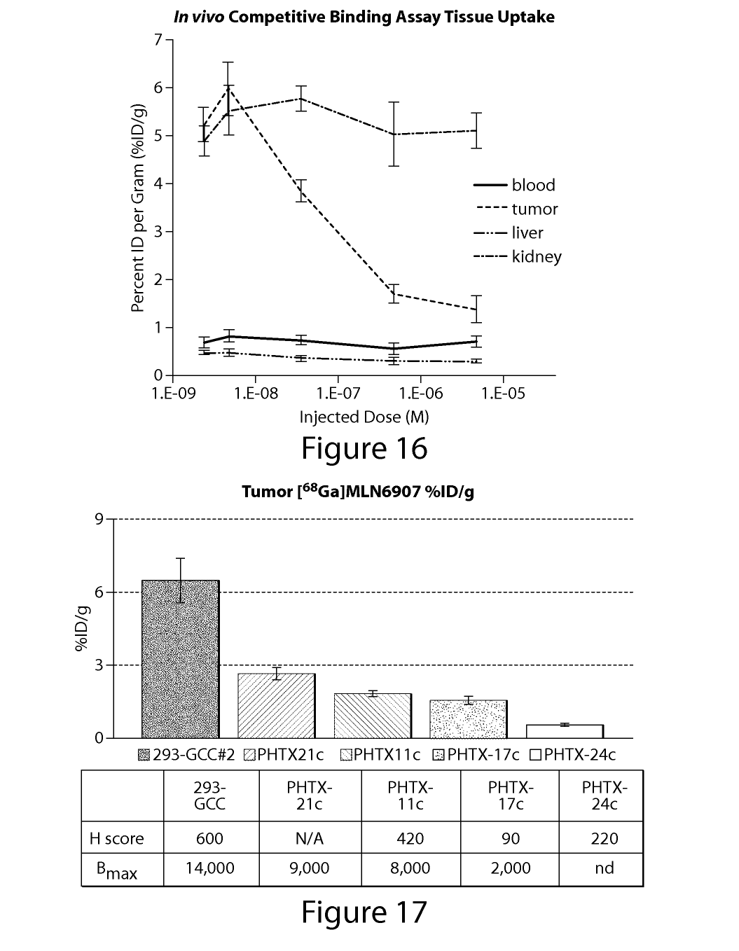

The K_{D} of MLN6907 for cell-surface GCC was calculated using an in vitro competitive binding assay with HEK-293 GCC clone 2 cells (FIG. 13). Antigen surface density of GCC on HEK-293 GCC clone 2 cells was estimated in vitro by Western blotting, and in vivo in the mouse model (FIG. 16). Decreased [68Ga]MLN6907 uptake in the tumor results from GCC receptor competition from added unlabeled peptide. The half-life of compound internalization was determined to be 56 minutes (FIG. 14). Blood pharmacokinetic data from the mouse model is shown in FIG. 15; MLN6907 clears rapidly from blood (t_{1/2}=26 min) through renal excretion and is retained in tumor tissue according to GCC antigen levels. The highest radiation exposure to [68Ga]MLN6907 was in the kidney and bladder indicating that renal excretion was a primary route of elimination. FIG. 17 shows the results of administering MLN6907 to mice bearing tumors with different GCC levels. The tumor uptake of [⁶⁸Ga]MLN6907 differed in tumors with varying levels of GCC expression and phenotype and is reflected in the initial in vivo calculated Bₘₐₓ values.

Using OLINDA/EXM software, the effective dose was estimated to be 0.0131 mSv/MBq (1 mCi=37 MBq) in adult human male and 0.0139 mSv/Mbq in adult human female.

### Example 9

### Safety Studies

MLN6907 DSP had no effect on central nervous system (CNS), cardiovascular (CV), or respiratory safety pharmacology endpoints evaluated in the GLP-compliant, 2-week, repeat-dose toxicology studies in Sprague-Dawley rats and cynomolgus monkeys.

The toxicology studies with MLN6907 DSP were designed to address the target tissues for toxicity, the dose-limiting toxicity, noninvasive methods for monitoring of clinically relevant toxicities, and the reversibility of adverse effects. Both rats and monkeys are pharmacologically responsive to the effects of the STp(5-18) peptide portion of MLN6907.

In GLP-compliant, 2-week, repeat-dose toxicology studies in Sprague-Dawley rats and cynomolgus monkeys in which MLN6907 DSP, at doses up to 0.25 mg/kg, was administered IV on Days 1, 4, 8, 11, and 14, with a 14-day recovery period, there were no findings indicative of toxicity. These studies support that, as a result of anatomic privilege, the normal intestine will not be a target for [68Ga]MLN6907 DP. In both studies, the no-observed-effect level (NOEL) was 0.25 mg/kg.

Toxicokinetics (TK) and immunogenicity of the MLN6907 DSP were evaluated as part of the GLP-compliant, 2-week, repeat-dose toxicology studies. There were no marked sex-related differences in exposure to MLN6907 DSP in Sprague-Dawley rats or cynomolgus monkeys. The maximum concentration (Cmax) and area under the concentration-versus-time curve from 0 to 24 hours (AUC0-24 hr) increased in an approximately dose-proportional manner and there was no marked accumulation of MLN6907 DSP in the plasma. The immunogenicity of the peptide component of MLN6907 was assessed by measuring rat and monkey antipeptide antibodies (rodent antipeptide antibody [RAPA] and primate antipeptide antibody [PAPA]). Briefly, antipeptide antibodies were detecting using FITC-STa and biotinylated-STa combined to capture anti-STa antibodies, then incubated and washed, followed by detection of the bound antibody with Streptavidin-Sulfo tag. There was no immunogenicity response at any dose.

Local tolerance of decayed nonradioactive [⁶⁸Ga]MLN6907 DP and MLN6907 DSP was evaluated using an in vitro hemolysis assay and as part of the GLP-compliant 2-week, repeat-dose toxicology studies, respectively. There was no erythrocyte hemolysis at decayed [⁶⁸Ga]MLN6907 DP concentrations of 3.5 μg/mL, nor were there any injection site findings in the rat and monkey 2-week toxicology studies using dose formulations up to 0.10 mg/mL.

The allowable MLN6907 DSP phase 1 clinical starting mass dose is 282 μg, which was calculated using the human equivalent dose of one-tenth of the NOEL (0.25 mg/kg) defined in the 2-week, GLP-compliant, repeat-dose toxicology study in Sprague-Dawley rats. This dose is greater than 67-fold the proposed human mass dose range of up to 43.4 μg on a mg/kg basis (Table 4-13) and is considered safe on the basis of the absence of toxicity after administration of multiple doses of 0.25 mg/kg in rats and monkeys.

### Example 10

### Human Pharmacokinetic Modeling

A PK/pharmacodynamic modeling and simulation analysis was used to estimate a superior dose range of [⁶⁸Ga]MLN6907 DP required to differentiate variable GCC expression levels in patient tumors. This analysis was performed using both blood and tumor uptake of [⁶⁸Ga]MLN6907 DP in the multiple tumor xenograft models after intravenous administration. This analysis was performed using PK and tumor uptake data from multiple tumor models including T84 (human colon carcinoma), human embryonic kidney (HEK)-293 GCC Clone 2, PHTX-17C (colon adenocarcinoma), and PHTX-21C (colon adenocarcinoma) xenografts. The parameter values for the modeling analysis are described in Example 8 above.

Tumor exposure kinetics were described using a Michaelis-Menten saturable effect compartment to illustrate the saturation of the GCC antigen with the increase of the [⁶⁸Ga]MLN6907 DP dose. The PK/pharmacodynamic exposure model was then coupled with a model of projected human PK and simulations were performed using this human PK/pharmacodynamic mathematical model to identify the relationship between [⁶⁸Ga]MLN6907 DP dose level and predicted tumor exposure. The fitted results demonstrated that the mouse PK/pharmacodynamic relationship can be mathematically well described as a saturable effect compartment model. The results suggest that a single clinical dose of [⁶⁸Ga]MLN6907 DP of <100 μg/person will provide excellent resolution of GCC antigen expression level without saturating GCC receptors.

### Example 11

### Clinical Trial Design

The radiolabeled compounds herein can be tested for safety and efficacy in imaging using the following protocol.

This study is a first-in-human (FIH), single-dose, open-label, phase 1 study designed to assess the safety, PK, distribution, and radiation dosimetry of the compound (such as [⁶⁸Ga]MLN6907) after a single intravenous (IV) administration. Additional exploratory objectives will include a qualitative evaluation of liver tumor localization of the compound (such as [⁶⁸Ga]MLN6907) and determining tumor-to-normal liver background ratios and kinetic parameters associated with [⁶⁸Ga]MLN6907 uptake, retention, and clearance in CRC liver metastases.

Patients with CRC who are scheduled for resection of liver metastases as part of their treatment plan will be eligible for enrollment. The planned surgery as scheduled for clinical management (i.e., not a study variable) should occur greater than 14 days after the day of imaging. The imaging study will include: 1) evaluation of safety, 2) whole body imaging to measure the organ and whole body dosimetry to evaluate radiologic exposure and safety, and 3) dedicated liver and liver metastases imaging for detailed measurement of tumor uptake, retention, and clearance of the compound (for instance [⁶⁸Ga]MLN6907).

The study will consist of a single IV administration of the compound (for instance [⁶⁸Ga]MLN6907), followed by PET imaging and qualitative and quantitative analysis of the PET images. The study will also investigate the relationship between GCC as visualized by [⁶⁸Ga]MLN6907 and as assessed by an analytically validated GCC IHC assay, which will be performed on tissue samples of the surgically resected liver metastases. Up to 20 patients will participate in the study. When available, the patients will consent to provide archival tumor tissue in previously resected tumor specimens (e.g., from the primary colon cancer) will be evaluated for GCC by IHC. Following the accrual of approximately 6 to 8 patients, exposure and kinetics will be assessed to determine if the dose and imaging conditions should be adjusted and the study should continue.

The activity of the compound (for instance [⁶⁸Ga]MLN6907) will be 4.0 to 6.0 (+10%) millicuries (mCi) of ⁶⁸Ga (up to 43.4 μg of peptide) as supported by nonclinical experimental and simulation data. The patients will undergo PET/computed tomography (CT) imaging. After IV administration of the compound such as [⁶⁸Ga]MLN6907 (a single bolus injection over 10 to 20 seconds), the first 6 patients will undergo dynamic whole body scanning for up to 120 minutes after injection. Whole body imaging will start at the skull vertex and end at the upper thighs according the following protocol: 1-minute, 2-minute, 3-minute, 4-minute, and 5-minute scans. Scan framesets will be overlapped by 5 mm. Patients will be encouraged to remain orally hydrated with water and to empty their bladder prior to being positioned on the scanner to ensure that scanning can be completed without voiding. In subsequent patients, dynamic scanning may be conducted over the liver in the region of the liver metastases for up to 120 minutes. With respect to live scans, at the beginning of the single IV injection, scan acquisition will commence according to the following protocol: 10-second images for 3 minutes; 20-second images from 3 to 5 minutes; 1-minute images from 5 to 10 minutes; 2-minute images from 10 to 20 minutes; 5-minute images from 20 to 60 minutes; and 10-minute images from 60 to 120 minutes or until the imaging stops due to patient constraints or other reasons.

Vital signs will be monitored for up to 8 hours after administration of the compound (such as [68Ga]MLN6907). Monitoring of adverse events (AEs), serious and nonserious, will be conducted throughout the study, including at an in-person safety assessment at Day 7. A final safety evaluation follow-up assessment will be performed by telephone approximately 14 days after the administration of the compound (such as [68Ga]MLN6907), before the scheduled liver surgery. Toxicity will be evaluated according to National Cancer Institute Common Terminology Criteria for Adverse Events (NCI CTCAE), version 4.03 (effective date 14 Jun. 2010).

Following the collection of image data on a clinical PET/CT system, data for each scan will be reconstructed on the system workstation to create relevant whole-body and dynamic tomograms. Image data will be transferred using the ACR Image Metrix TRIAD platform. This platform will be installed locally at the imaging center. Imaging data will be processed and evaluated. Images of adequate quality and tumor visibility will be then subjected to quantitative analysis. If the initial qualitative visual analysis of the liver images demonstrates liver tumors that are rated by an expert reader as at least a 3 on a 1 to 5 scale (1=definitely identifiable tumor uptake; 2=probably identifiable tumor uptake; 3=equivocal; 4=poorly identifiable; 5=definitely nonidentifiable tumor uptake; and N/A=not imaged), the images will undergo quantitative analysis and the study will proceed. The images may undergo PET pre-processing. Images may be spatially smoothed using a Guassian kernel of 5 to 20 mm prior to subsequent analysis depending on the inherent count density and noise in the raw images and time-activity curves. The time-activity curves may also be temporally smoothed depending on the noise properties required for subsequent analysis using Logan, curve fitting, and other analyses. Images from different time points may be summed over time to simulate static image acquisition and to generate images for the qualitative analyses. The precise application of spatial and temporal smoothing and the time interval for image summing will be made based on the initial image results. Quantitative analysis will include ROI analysis, SUV determination, time-activity curve (TAC) generation, Logan plots, and tracer kinetic modeling.

After surgical resection, the uptake of the compound (such as [⁶⁸Ga]MLN6907) will be compared with GCC expression in liver metastases, as assessed by a validated IHC analysis of the resected surgical specimens.

This study will also help to determine the time points after injection that result in the highest uptake of the compound (such as [⁶⁸Ga]MLN6907) in metastatic lesions and the best tumor-to-normal liver background ratio by visual and quantitative analysis. In addition, the study will build on the pharmacokinetic data collected to date by directly determining the PK profile of the compound (such as [⁶⁸Ga]MLN6907 and MLN6907 DSP) and quantify the compound (such as [⁶⁸Ga]MLN6907) binding to GCC using tracer kinetic modeling.

The study will also include an assessment of the correlation between GCC detected via PET and via IHC.

Patients will be males or females 18 years or older with a diagnosis of CRC and evidence of respectable liver metastases. Patients will have an Eastern Cooperative Oncology Group (ECOG) performance status of 0 to 1 and will be scheduled for resection of liver metastases as part of their treatment plan no less than 14 days after the administration of [68Ga]MLN6907.

### Example 12

### Image Data Analysis

All activity concentrations including, but not limited to, visible activity, blood activity, bone activity, red marrow activity, and excreted activity will be presented in a by-patient listing with percent of injected dose and injection time by the visible organ as seen on the images, and plotted with time after injection. All the parameters from the radiation dose calculation will be included in a separate by-patient listing.

The number and percentage of the visual rating using a 5-point scale will be tabulated and quantitative measurements of tumor-to-background ratio for each liver tumor will be descriptively summarized. Time to determine the signal-to-background ratio and optimal imaging time point to get the maximum ratio for each tumor lesion will be presented in a by-patient listing.

All images will first be evaluated for overall image quality, including for motion artifact, attenuation artifact, and noise. Activity at the injection site, if within the FOV, will be evaluated for evidence of dose infiltration. High-quality images will then undergo analysis for radiation dosimetry calculation and liver images will be interpreted and rated for tumor uptake. The whole body image acquisition protocol will result in 5 time points, including the liver and liver tumors. For 8 image plane sets from the skull vertex to the upper thighs, the mid-acquisition time points for the liver images will be approximately 4, 16, 36, 64, and 100 minutes after injection. If the protocol is modified after the initial few patients in order to increase the imaging time for the liver, the midliver image time points will be slightly different. For example, if the compound (e.g., [⁶⁸Ga]MLN6907) uptake and clearance kinetics are such that the tumor/normal liver contrast is greatest at the 16- and 36-minute images, then the 2 images can be summed for interpretation and rating. Images will be presented to the expert reader with and without co-registered CT, MRI, or FDG-PET images for review. Images will be presented at a standard contrast and brightness for review on an appropriate image workstation. Individual tumor foci seen on PET images will be graded on a 5-point scale regarding the presence or absence of the compound (such as [⁶⁸Ga]MLN6907) uptake: 1=definitely identifiable tumor uptake; 2=probably identifiable tumor uptake; 3=equivocal; 4=poorly identifiable; 5=definitely nonidentifiable tumor uptake; and N/A=not imaged. Tumor uptake heterogeneity will also be evaluated according the following scale: 4=highly heterogeneous; 3=moderately heterogeneous; 2=slightly heterogeneous; 1=homogenous; N/A=not imaged. PET scan tumor heterogeneity will be used when correlating PET and IHC results.

The dedicated, kinetically acquired liver images will be evaluated qualitatively and quantitatively. Due to the number of time points and images acquired kinetically for the liver and liver tumors, static images will be generated by summing the kinetic images over defined time intervals. The time intervals will be chosen from the quantitative ROI liver tumor and normal liver (or muscle or blood) TACs and liver tumor/normal liver (or muscle or blood) ratios as function of time. Two time intervals will be identified from the quantitative analysis and employed in the qualitative visual analysis for comparison. Signal (e.g., [⁶⁸Ga]MLN6907) images will be displayed and interpreted with and without co-registered CT, MRI, or FDG-PET images in order to compare liver tumor detection with and without a priori anatomic information on liver tumor number, size, and location. Individual tumor foci seen on PET images will be graded on a 5-point scale regarding the presence or absence of compound (e.g., [⁶⁸Ga]MLN6907) uptake: 1=definitely identifiable tumor uptake; 2=probably identifiable tumor uptake; 3=possible or equivocal uptake; 4=poorly identifiable; 5=definitely nonidentifiable tumor uptake; and N/A=not imaged. Tumor uptake heterogeneity will also be evaluated according the following scale: 4=highly heterogeneous; 3=moderately heterogeneous; 2=slightly heterogeneous; 1=homogenous; N/A=not imaged.

Regions of interest (ROIs) will be identified on the primary organs from the whole body imaging and the time-activity curves computed (without radioactive decay correction). The area under the curve will be computed for each organ and for residual activity. Activity after the last time point will be assumed to be further lost by radioactivity decay alone. These data will be entered into the OLINDA/ECM software for calculation of organ and whole body effective dose equivalents in male and female patients.

For kinetic analysis of uptake and kinetics of normal liver and liver metastasis, ROIs will be identified in the center of each liver metastasis and on an appropriate region of adjacent liver tissue away from the liver metastases and the liver edge. ROIs will also be identified on other abnormal foci in the whole body images. An abdominal aorta ROI will also be identified for generation of blood kinetics. Summed images will be used for this purpose for better anatomic resolution, together with CT images if liver lesions are visible without contrast. ROIs will be transferred to each imaging time point and the kinetic curves will be generated.

Furthermore, data will be exported for parameter estimation (e.g., antigen density, vascularity, and other endpoints relevant to compound (e.g., [⁶⁸Ga]MLN6907) delivery and uptake) that may be determined through mechanistic modeling.

Images will be evaluated by an external bioimaging expert. Overall image quality will be graded as adequate or inadequate. For all images assessed as inadequate, the reason will be specified. Individual tumor foci seen on PET and/or CT will be graded on a 5-point scale regarding the presence or absence of adequate compound (e.g., [⁶⁸Ga]MLN6907) uptake: 1=definitely identifiable tumor uptake; 2=probably identifiable tumor uptake; 3=equivocal; 4=poorly identifiable; 5=definitely nonidentifiable tumor uptake; and N/A=not imaged. For all foci (primary and metastases, if identified) interpreted as grade 3 or lower on the unblended reading, an SUV calculation (or tumor/normal liver, tumor/muscle, etc.) will be performed. These parameters will be evaluated as a function of time to determine the signal-to-background ratio and the optimal imaging time point.

To account for differences in liver metastasis size and for the PET-IHC correlation, a PVC will be applied using the phantom data and the measured size of each metastasis. Hot spot recovery and cold spot spill-in will be computed and a corrected value will be used in subsequent analyses. A similar calculation will be performed for the abdominal aorta.

Results, including the tumor/normal liver ratios and model fit data, will be summarized for all patients and mean values tabulated. The optimal time for imaging the tumor/normal ratio will be determined. The tumor/normal liver ratio will be plotted against the full model approach and the relationship evaluated for correlation. A mechanistic model for the purposes of studying transport and kinetics of radiolabeled agents in tumors will be used.

This mechanistic model has been used as a predictive tool for study design as well as a novel means of estimating in vivo tumor parameters (e.g., Bₘₐₓ and vascularity). Visual rating measurements will also be used in separate analyses.

Quantitative liver analysis will involve sphere phantom data. Phantom data will be corrected for attenuation and for radioactivity decay from the time of calibration of the radioactive solution (μCi/ml) used to fill the phantom. The same solution will be imaged in a uniform phantom of at least 5 times the PET scanner full width at half maximum (FWHM) in order to ensure full recovery. ROI analysis will be conducted using: 1) a 3×3 ROI placed in the center of each sphere, and 2) an isocontour ROI defined by the sphere border from the PET CT scan. Max pixel and 3×3 pixel ROIs centered on the max pixel (described below) may be located eccentrically on the tumor, and so would require corresponding placement on spheres; this approach will be reserved for a secondary analysis depending on results for tumor imaging. Larger or smaller ROIs may be used depending on the initial results and the performance characteristics of the specific PET scanner employed. ROIs will be placed on hot and cold spheres. The same ROIs will be placed on the corresponding uniform phantom. The uniform and hot/cold sphere phantoms will be decay corrected to the same time for quantitative comparison. The hot spot recovery coefficient (HSRC) defined by ROI measured activity in each sphere to that obtained in the uniform phantom (HSRC=ROI^{htsp}/ROI^{uni}) and cold spill-in fraction (CSRC=ROI^{cdsp}/ROI^{uni}) will be similarly calculated for each sphere. The data will be plotted for the different sized spheres and a curve will be fit to the HSRC and CSRC values. These curves can then be used to calculate the PVC liver tumor values for any tumor volume. For highly irregular and heterogeneous tumors addition methodology may be employed, such as pixel-by-pixel PVC, or such tumors may be eliminated from analysis.

Several approaches to ROI analysis will be undertaken for purposes of comparison and for regression analysis with the IHC GCC results. The IHC data may be representative of the average GCC throughout each liver tumor or may be more representative of specific sampled subregions of each liver tumor. Therefore, the ROI analysis must encompass the various possibilities. Accordingly, ROI analysis will include: 1) maximum tumor pixel; 2) 3×3 pixels centered on the maximum pixel; 3) 3×3 pixels placed on tumor center; and 4) isocontour defined by co-registered CT or MRI. Other sized ROIs may also be employed depending on the initial results. The ROIs will be converted to body mass SUV (SUV_{bm}) and lean body mass SUV (SUV_{lbm}). For the PVC analysis, a circumferential region will be drawn around each tumor for average background determination for the CSRC calculation. The region will be a 2-pixel thick annular region whose inner border is positioned approximately 1 FWHM (final imaging resolution) from the outer tumor border as shown on the CT or MRI scan. A 3×3 ROI will be placed on the abdominal aorta behind the liver on 3 adjacent slices and an annular ROI will be placed around the aorta. Three 3×3 ROIs will be placed in 3 regions of normal liver at least 2 cm from any tumor, the liver edge, or any large vascular structures. Three 3×3 ROIs will be placed in the muscle of the abdominal wall adjacent to the liver or on another appropriate muscle region as demonstrated on CT image and the 3 ROIs will be averaged for subsequent analysis. Other sized ROIs may also be employed depending on the initial results.

The diameters of individual liver tumors will be measured in the x, y, and z dimensions using the best available anatomic images (CT or MRI) for edge detection and tumor definition. Anatomic images should be acquired within 1 month of the PET scan for size correspondence. The volume of each tumor will be calculated using the formula for the volume of a sphere (V=4/3πr3) and used for PVC and for analysis of the visual rating results as a function of tumor size.

Partial volume correction (PVC) will conducted on summed images from the dedicated liver imaging protocol, for example, for 20 to 30 minutes of data, at a time point where the tumor/normal liver (or muscle/blood) is maximal (to be determined from the quantitative ROI analysis). Dynamic PVC could be undertaken based on the initial kinetic data results; this is a more complex analysis. PVC may also be undertaken for the static liver images from the whole body imaging as justified by the image quality; summed images may also be used, as described elsewhere. PVC of liver tumor compound (e.g., [⁶⁸Ga]MLN6907) uptake will be accomplished using the individual tumor ROI values, the tumor volumes computed from the CT or MRI scans, and the HSRC and CSRC values from the sphere phantom data. The tumor-size appropriate spill-in contribution to tumor uptake will first be subtracted (on a per-pixel basis) using the annular background region for each tumor. Next, final corrected tumor uptake value will be calculated by dividing by the HSRC. The formula for the i^{th} tumor is

$${T}_{i}^{PVC} = \frac{1}{{HSRC}_{i}} [ {T}_{i} - ( {CSRC}_{i} \times {Bkg}_{i} )$$

where Bkgᵢ is the background around the i^{th} tumor. The aorta activity will also undergo PVC for the summed image. For highly irregular and heterogeneous tumors addition methodology may be employed, such as pixel-by-pixel PVC, or such tumors may be eliminated from analysis.

Time-activity analysis will be performed. Time-activity curves (TACs) will be generated by plotting the individual tumor decay-corrected ROI results as a function of time. Separate curves will be generated for the different ROI placement methods described above. The aorta blood and muscle TACs will be generated similarly. Curves for sampled venous blood will be generated. Ratio curves will be generated as a function of time for liver tumor/normal liver, liver tumor/muscle, and liver tumor/blood. When this information is available, aorta ROI values, and venous blood samples, will be used for blood values. Blood values will be used with and without correction for the percentage radiolabeled metabolites as a function of time, when this information is available.

Kinetic modeling will be performed. PET and blood activity will first be converted to nM using the activity concentrations (μCi/ml) and the specific activity calibrated to the time of injection. The plasma input data from the PVC aorta will be corrected for radiolabeled metabolites and protein binding to generate the free plasma compound (e.g., [⁶⁸Ga]MLN6907) input function. Modeling analysis will be performed using Logan plots and by compartmental modeling using 1-tissue and a 2-tissue models. A fixed liver percentage blood volume of 25% and tumor volume from the best available data will be used in all modeling evaluations. If fixed blood volume values are unavailable or unreliable, the tumor blood volume may be floated as an estimated parameter; the magnitude of the required blood volume correction will be determined by comparing blood and tumor activity. The 1-tissue model has 2 rate constants (K₁, k₂) that will be fitted to determine volume of distribution (VT), VT=K₁/k₂. The 2-tissue model has 4 rate constants (K₁, k₂, k₃, k₄) that will fitted to determine VT=K₁/k₂ (1+k₃/k₄). With the Logan method, a bilinear form of the equation will be used, with VT computed using the transformed data from approximately 30 to 120 minutes or to the end of the study. Time intervals of 30 to 60 and 30 to 90 minutes will also be used for comparison and for determination of best linearity. Compartmental modeling will also be performed with stopping times of 60 and 90 minutes; the results will be compared to evaluate the effect of study duration. The F-test and Akaike information criterion will be used for model fit evaluation. Logan analysis will also be performed with muscle as the input and the results will compared to those using blood as the input. Similarly, 1- and 2-tissue fits will be performed for muscle and the K₁ and k₂ values and K₁/k₂ ratios compared to those in the tumors. Modeling will be performed separately for each tumor in which compound (e.g., [⁶⁸Ga]MLN6907) uptake is significant as judged by the qualitative analysis. Analysis will be performed using the plasma input function from the imaged PVC aorta input function. The results will be tabulated and compared across all tumors for variability of Logan slope values and for K₁, k₂, k₃, and k₄. If relative constancy of K₁/k₂ across the tumors is observed, then model fits will be performed with K₁/k₂ fixed and the k₃, k₄, and k₃/k₄ values compared to the fits with all parameters floating. The k3/k4 ratios and Logan slopes will be compared across tumors for a given patient and across all tumors to the tumor/normal liver, tumor/blood, and tumor/muscle ratios over the various time intervals to determine the degree of correlation and value of the simple ratios versus the more complex modeling analysis. The time interval over which the tissue ratios best correspond to the modeling analysis will be noted.

Quality control and phantom studies will be performed. The following example uses ⁶⁸Ga, but it is understood that another radioactive atom could be substituted if the intended imaging agent uses another radioactive atom. In the morning of each imaging day, a uniform cylindrical phantom approximately 20 cm in diameter and 20 cm long will be imaged to determine uniformity and calibrate the well calibrator for blood sample analysis. A volume-calibrated sample from the uniform phantom will be counted in the well counter. The start/stop times for the uniform phantom imaging and the sample counted in the well counter will be noted for decay correction. Additional monthly, weekly, and daily PET scanner quality control procedures will have been completed, reviewed, and passed prior to initiation of compound (e.g., [⁶⁸Ga]MLN6907) imaging. Prior to study initiation and after every 5 patients, a phantom with 6 hollow spheres ranging from 5 to 30 mm diameter will be imaged. Prior to imaging of the sphere phantom, a 20 cm uniform phantom filled with a ⁶⁸Ga solution will be imaged. Immediately after imaging, the individual hollow spheres will be filled with the same ⁶⁸Ga solution used in the uniform phantom. The space outside the hollow spheres will be filled with nonradioactive water. The phantom will be placed in the center of the PET scanner FOV and imaged. After imaging, the hollow spheres will be emptied, cleaned, and filled with nonradioactive water. The space around the spheres will then be filled with the ⁶⁸Ga solution used in the uniform phantom and the phantom will then be imaged. The start/stop times for the uniform phantom, the hot sphere phantom, and the cold sphere phantom will be noted for decay correction. These data will permit calculation of the hot spot and cold spot recovery coefficients for PVC of different sized liver tumors.

Furthermore, data may be exported for parameter estimation (e.g., antigen density, vascularity, and other endpoints relevant to [68Ga]MLN6907 delivery and uptake) that may be determined through mechanistic modeling.

Visual rating measurements will also be used in separate analyses.

PET data output can be correlated with IHC data. To account for differences in liver metastasis size and for the PET-IHC correlation, a PVC will be applied using the phantom data and the measured size of each metastasis. Hot spot recovery and cold spot spill-in will be computed and a corrected value will be used in subsequent analyses. A similar calculation will be performed for the abdominal aorta.

Results, including the tumor/normal liver ratios and model fit data, will be summarized for all patients and mean values tabulated. The optimal time for imaging the tumor/normal ratio will be determined. The tumor/normal liver ratio will be plotted against the full model approach and the relationship evaluated for correlation. A mechanistic model for the purposes of studying transport and kinetics of radiolabeled agents in tumors will be used. This mechanistic model has been used as a predictive tool for study design as well as a novel means of estimating in vivo tumor parameters (e.g., Bₘₐₓ and vascularity).

1. The tumor/normal liver, tumor/muscle, and tumor/blood ratios at an appropriate early and late time interval as determined from the TACs and ratio curves such that the ratio is high at both time intervals. Both the partial volume-corrected (PVC) 3×3 ROIs and contour ROIs will be used.

2. Logan slopes for 30- to 120-minute (or study end) and 30- to 60-minute intervals using the PVC 3×3 and contour ROIs

3. The 2-tissue compartment k₃/k₄ values PVC from the 4-parameter float results and from the results with fixed K₁/k₂ ratios from the muscle data and from the average tumor K₁/k₂, as appropriate.

4. Antigen density (Bₘₐₓ) and vascularity as determined by the mechanistic model.

### Example 13

### Preclinical Imaging Studies

Here we describe a methodology of PET/CT study design for a radiolabeled compound, e.g [68Ga]MLN6907, based on a combination of in vitro, ex vivo, and in vivo preclinical imaging studies and model-based estimation of tumor parameters from simulated clinical PET data. In a series of experiments, the peptide affinity, internalization rate, and clearance were determined in patient-derived CRC xenografts with varied tumor microenvironmental phenotype. In addition to supporting the clinical development of the imaging agent, this data was used in combination with simulated clinical list-mode PET data to evaluate estimation of tumor parameters under several clinically viable acquisition and reconstruction conditions. Specifically, liver CRC metastases of varying tumor diameter, antigen density, and vascularity were simulated in combination with PET imaging acquisition duration and reconstruction with and without partial-volume correction. Tumor, liver, and background time-activity curves (TACs) were generated from the reconstructed data and analyzed using a distributed tumor model to estimate the known tumor antigen density and vascularity. Analysis of the simulation studies revealed: 1) Partial volume correction helps to achieve superior antigen density and vascularity estimation; 2) Parameter estimation was most accurate within a tumor size range of 1-5 cm; 3) Parameter estimation was robust for all tested TAC reconstruction durations (e.g., 2, 3, 5, and 10 min); 4) Parameter estimation was optimal for common clinical acquisition times of 30-90 minutes; 5) Antigen density estimation was less accurate in poorly vascularized tumors.

Partial volume effect (PVE) correction is routinely used in PET imaging to improve quantification accuracy resulting from low spatial resolution and sampling errors (i.e., estimating round objects spatially with rectilinear voxels). Application of a PVE method typically depends upon the available data and software and has most extensively been studied for ¹⁸F-FDG tumor imaging with SUV and/or volume as the primary readout. In this study of [⁶⁸Ga]MLN6907, PVE correction was evaluated in terms of its ability to improve estimation of tumor antigen density and tumor vascularity via application of a mechanistic pharmacokinetic (PK) model to measured time-activity curves (TACs). Specifically, TACs for liver colorectal cancer metastases of varying tumor volume, antigen density, and vascularity were generated. These TACs were generated using a mechanistic PK model that incorporated previously measured values of affinity, internalization rate, and clearance for [⁶⁸Ga]MLN6907. Liver and background TACs were generated based on preclinical imaging evaluation in rats and from the XCAT human digital phantom. Multiple simulated lesions were placed within the liver to complete the digital phantom. Simulated TACs and lesion-bearing phantoms were integrated into a model of a clinical PET scanner (AnyScan PET/CT, Mediso Kft.) to generate four hours of simulated list-mode data. List-mode data were reconstructed over multiple acquisition windows (e.g., 0-60 min, 30-90 min, etc.) and time frame intervals (e.g., 2 min, 3 min, etc.). Lesion TACs were extracted from reconstructions using the known lesion location (CT data will serve this purpose for clinical studies). The geometric transfer matrix (GMT) method of PVE correction was applied to correct for spillover from the liver (spillover from the background ROI was found to be negligible given lesion placement). A 3D Gaussian kernel with full-width at half maximum (FWHM) values ranging from 3-30 mm was used to represent the system point spread function. Resulting corrected and uncorrected TACs were analyzed using the PK model described above to estimate tumor antigen density and vascularity. PVE correction was found to be essential for accurate estimation of tumor vascularity and tumor antigen density, particularly because of the need to correct for high liver signal intensity at early time points. Optimal performance was observed in a range of 15-18 mm FWHM with lower values in this range tending to yield better estimates of vascularity and higher values in this range yielding better estimates of antigen density.

### Example 14

### Assessing the Biodistribution and Tolerability of Bismuth-213 Labeled STp(5-18) Peptide in Naïve and Tumor Bearing Mice

A biodistribution study was first conducted in 24 female mice to observe the ex vivo biodistribution of ²¹³Bi-DOTA-Ahx-STp(5-18). All tumor bearing (GCC) mice were randomized into n=4×3 groups and all naïve mice were randomized into n=4×3 groups. Due to issues with the gamma counter device, some data was lost, reducing the study to 20 animals total (n=12 tumor bearing; n=8 naïve). All mice were administered ²¹³Bi-DOTA-Ahx-STp(5-18) intravenously (IV) and animals were sacrificed at the following time points post injection: 15 minutes, 1 and 3 hours. The following tissues were resected for gamma counting to determine the tracer-derived radioactivity per unit tissue: tumor, blood, heart, kidney, liver, lungs, spleen, pancreas, bladder, small intestine, large intestine, stomach, bone, and muscle.

### Labeling Protocol

First, an Ac-225 generator was washed with 3 mL 0.01 M HCl. Once washed, ²¹³Bi was eluted with 0.8 mL of NaI/HCl 0.1 M/0.1 M. The generator was then washed a second time with 3 mL of 0.01 M HCl and stored with 0.01 M HCl to prepare for subsequent ²¹³Bi elutions. The elution process was started using a flow rate of 0.15 mL per minute using a Cole Parmer pump set at a speed of 4.0. Concurrently the DOTA-Ahx-STp(5-18) compound (0.1 μg/μL 6/23/14 and 1.0 ug/uL, 6/25/14 and 6/27/14) was warmed at 60° C. for 10 minutes. After the incubation period the following components were added to the reaction mixture to a volume of approximately 1 mL (pH 8.5 to 8.8): ²¹³Bi, 2 M Tris buffer, and 20% Ascorbic Acid. The reaction was then heated for 5 minutes at 100° C. Labeling efficiency was confirmed using SG-ITLC with 0.1 M citrate buffer. Incorporation ranged from 96 to 98%. Prior to administration, 5 μL of DTPA was added to the reaction vial and sterile PBS for a total of 100 μL per injection per animal.

### Study Design

The ex vivo biodistribution of ²¹³Bi-DOTA-Ahx-STp(5-18) was observed in 24 female mice. All tumor bearing (GCC) mice were randomized into n=4×3 groups and all naïve mice were randomized into n=4×3 groups. Due to issues with the gamma counter device, some data was lost, reducing the study to 20 animals total (n=12 tumor bearing; n=8 naïve). The animals in group 1 (tumor-bearing) weighed 19.8±1.91 g and the animals in group 2 (control) weighed 18.7±1.61 g at the start of the study. All mice were administered ²¹³Bi-DOTA-Ahx-STp(5-18), IV. A subset of animals from each group was sacrificed at each of the following time points post injection: 15 min., 1, and 3 hours. The following tissues were resected for gamma counting from each carcass: tumor, blood, heart, kidney, liver, lungs, spleen, pancreas, bladder, small intestine, large intestine, stomach, bone, and muscle.

### Biodistribution Study Design Summary

| | | | | Radio- | |
|---|---|---|---|---|---|
| | | | | activity | |
| Group | No. | | Mass Dose | Dose | |
| & | Female | | (μg/ | (μCi/ | Time Points |
| Route | Animals | Tracer | animal) | animal) | (h) |
| 1 IV | 12 | ²¹³Bi- DOTA- | 0.142 ± 0.04 μg | 31.2 ± 8.60 μCi | 0.25 (n = 4) |
| | | Ahx-STp(5-18) | | | 1 (n = 4) |
| | | | | | 3 (n = 4) |
| 2 IV | 8 | ²¹³Bi- DOTA- | 0.191 ± 0.00 μg | 38.4 ± 4.54 μCi | 0.25 (n = 3) |
| | | Ahx-STp(5-18) | | | 1 (n = 3) |
| | | | | | 3 (n = 2) |

Following completion of the biodistribution study, an in vivo tolerability study was conducted in female tumor-bearing GCC-293 mice. Initially, 60 tumor bearing mice were randomized based on tumor size into one of four groups. Each group (n=8 animals per group) was characterized by its drug intervention and/or dose. Animals assigned to group 1 were administered 3 doses of 140 μCi ²¹³Bi-DOTA-Ahx-STp(5-18), intravenously (IV) every other day (Q2D) on days 0, 2 and 4. Animals in group 2 were administered 3 doses of 50 μCi ²¹³Bi-DOTA-Ahx-STp(5-18) IV Q2D on days 0, 2, and 4. Animals in group 3 were administered vehicle (0.1 M HCl/0.1 NaI, Tris, ascorbic acid, ultra-pure water, ethanol, and DTPA in exact proportion to labeled drug brought up in PBS; 100 μL) IV Q2D on days 0, 2, and 4 and served as negative controls. Lastly, animals in group 4 were administered 3 doses of 7.5 mg/kg of a GCC-targeting antibody drug conjugate once weekly for three weeks for a total of 3 doses on days 0, 7 and 14 and served as positive controls. Following dosing, tumor caliper measurements and body weights were recorded three times a week for 21 days or until animals were sacrificed (animals were sacrificed if tumor size exceeded 2000 mm³ or body weight loss exceeded 20%). At the time of sacrifice, select tissues were collected from each animal and fixed in formalin for potential future IHC analysis.

### Tolerability Study Design Summary

| | | | Radio- | |
|---|---|---|---|---|
| | | | activity | |
| Group | No. | | Dose | |
| & | Female | | (μCi/ | |
| Route | Animals | Dose Agent | animal) | Mass Dose |
| 1 IV | 8 | ²¹³Bi-DOTA- | 110.1 ± 11.5 μCi | 0.017 mg/kg |
| | | Ahx-STp(5-18) | (D 0) | (D 0) |
| | | | 120.2 ± 20.8 μCi | 0.020 mg/kg |
| | | | (D 2) | (D 2) |
| | | | 118.1 ± 9.22 μCi | 0.021 mg/kg |
| | | | (D 4) | (D 4) |
| 2 IV | 8 | ²¹³Bi-DOTA- | 43.70 ± 3.78 μCi | 0.010 mg/kg |
| | | Ahx-STp(5-18) | (D 0) | (D 0) |
| | | | 51.40 ± 7.82 μCi | 0.013 mg/kg |
| | | | (D 2) | (D 2) |
| | | | 40.70 ± 13.5 μCi | 0.014 mg/kg |
| | | | (D 4) | (D 4) |
| 3 IV | 8 | Vehicle | NA | NA |
| 4 IV | 8 | GCC-ADC | NA | (3x) 7.5 mg/kg |
| | | | | QW (D 0, D 7, |
| | | | | D 14) |

### Analysis

All resected tissues from the biodistribution study were analyzed by EG&G Wallac 1480 Wizard 3″ gamma counter to determine the ²¹³Bi-DOTA-Ahx-STp(5-18) derived radioactivity. The following tissues were analyzed: tumor, blood, heart, kidneys, liver, lungs, spleen, pancreas, bladder, small intestine, large intestine, stomach, bone, and muscle. Data were recorded in units of counts per minute (CPM) and later converted to units of μCi for presentation in activity concentration units of percent injected dose per gram (% ID/g).

$$μ Ci = \frac{( CPM / Efficiency )}{2.22 * {10}^{6}}$$

Efficiency values were calculated from 10- and 100-fold or 50- and 500-fold stock dilutions counted in triplicate for each day and/or assay time and averaged.

Tumor caliper measurements were recorded two times a week to estimate tumor volumetric changes post dosing for animals in the tolerability study. Tumor caliper measurements were recorded in units of mm and later converted to a unit of volume, mm³, and fold change was calculated using the equations below:

$$Tumor Volume ( {mm}^{3} ) = \frac{( length * {width}^{2} )}{2}$$

$$Fold Change = \frac{{V}_{{t}_{i}}}{{V}_{{t}_{o}}}$$

For experiment 1, data was lost for some naïve animals due to gamma counter malfunction. As a result, the total number of animals on study was reduced to 20 (as opposed to 24).

For experiment 2, any animal whose tumor grew greater than 2000 mm³ or body weight decreased greater than 20% were euthanized in extremis.

### Results of Biodistribution Study

The STp(5-18) peptide was successfully labeled with ²¹³Bi but some peptide hydrolysis was observed over time. This type of radiolysis can be mitigated by the addition of stabilizers (ethanol etc).

Naïve and tumor-bearing mice demonstrated similar tissue uptake of ²¹³Bi-DOTA-Ahx-STp(5-18) over a 3 h time period. Highest uptake was seen in tumor, kidney, and bladder due to high renal excretion as expected. These values are similar with uptake and clearance seen with ⁶⁸Ga-DOTA-Ahx-STp(5-18).

²¹³Bi-DOTA-Ahx-STp(5-18) clears rapidly from blood (t_{1/2}=36-51 min) through renal excretion and is retained in tumor tissue.

Fold uptake of ²¹³Bi-DOTA-Ahx-STp(5-18) in specific tissues compared with tumor are also in par with ⁶⁸Ga-DOTA-Ahx-STp(5-18).

The switch from ⁶⁸Ga to ²¹³Bi as the chelating radiometal did not change the biodistribution in a mouse model. Nephropathy due to high kidney uptake can be mitigated by the co-administration of a kidney protectant.

### Results of Tolerability Study

The High Dose cohort treated with 3 doses of 140 μCi ²¹³Bi-DOTA-Ahx-STp(5-18) was not tolerated well and required euthanasia of all mice over a 14 day period due to body weight loss of >20%. The Low Dose cohort treated with 3 doses of 50 μCi ²¹³Bi-DOTA-Ahx-STp(5-18) had improved tolerability. No issues were observed with a GCC-targeting antibody-drug conjugate used as a positive control.

On average, lower tumor growth was observed up to day 15 for the high and low dose cohorts. Using this approach, statistical significance was seen with the high dose at day 11. The positive control was efficacious with a T/C=0.16 (day 22).

### P-Values for Tumor Growth

| | High Dose Vs. | Low Dose Vs. | Positive Control vs |
|---|---|---|---|
| Day | Negative Control | Negative Control | Negative Control |
| 3 | 0.30 | 0.38 | 0.25 |
| 8 | 0.068 | 0.17 | 0.0022** |
| 11 | 0.016* | 0.20 | 0.0092* |
| 15 | NA | 0.17 | 0.021* |
| 18 | NA | 0.66 | 0.093 |
| 22 | NA | 0.52 | 0.00024** |
| T/C | NA | 1.04 (Day 22) | 0.16 (Day 22) |

Statistical significance calculated by two-tailed t-tests for independent samples.
NA; Not applicable, Animals were euthanized prior to designated time point
*p < 0.05
**p < 0.005

Tumor volume was also calculated as tumor growth fold change from baseline to reduce tumor volume variability as discussed above. Statistical significance was seen for all treatment groups when compared with control. Also, T/C calculations showed significant reduction in tumor growth kinetics for all treatment groups.

### P-Values for Fold Change

| | High Dose Vs. | Low Dose Vs. | Positive Control vs |
|---|---|---|---|
| Day | Negative Control | Negative Control | Negative Control |
| 3 | 0.047* | 0.15 | 0.082 |
| 8 | 0.0025** | 0.75 | 0.000063** |
| 11 | 0.0023** | 0.91 | 0.00087** |
| 15 | NA | 0.00033** | 0.00043** |
| 18 | NA | 0.0010** | 0.00000024** |
| 22 | NA | 0.033* | 0.0000018** |
| T/C | 0.24 (day 11) | 0.95 (day 11) | 0.048 (Day 22) |
| | | 0.52 (Day 22) | |

*p < 0.05
**p < 0.005

Low dose and the positive control demonstrated a higher probability of survival compared with the negative control (Kaplan Meier plot for survival probability).

The high dose treatment (3×140 μCi ²¹³Bi-DOTA-Ahx-STp(5-18)) was not tolerated well, most likely due to dose limiting renal toxicity. Radiation nephropathy results in necrosis, atrophy and sclerosis following exposure of the kidney to ionizing radiation.

### Dosimetry: Imparted Dose

| | AUC | S factor | | |
|---|---|---|---|---|
| Organ | (h) | mGy/MBq-s | mGy/MBq | mGy* |
| Tumor | 0.068 | 0.08 | 20.7 | 322 |
| Kidney | 0.042 | 0.52 | 77.9 | 1220 |
| Blood | 0.162 | 0.06 | 35.8 | 556 |

*3 × 140 uCi doses on days 0, 2, and 4

Further MTD studies could include blood and urine sampling to analyze renal and hematological toxicity. A contemplated approach to reduce kidney uptake is the coadministration of a renal protectant (e.g., Clinisol, L- or D-Lysine, lysine/arginine, Gelofusine and amifostine) which changes the charge of renal tubules and decreases the absorption of peptides and free ²¹³Bi. By using a renal protectant, the maximum tolerance dose (MTD) could be increased and nephropathy reduced.

A statistically significant reduction in tumor growth kinetics was observed when looking at tumor growth fold change from baseline, comparing treated and control cohorts. This was also reflected in the calculated T/C values. Dose escalation of ²¹³Bi-DOTA-Ahx-STp(5-18) resulted in a lower T/C. Lower dose cohorts also demonstrated a higher probability of survival compared with the negative control.

### Example 15

### Assessing Effect of Renal Protectants on ²¹³Bi-DOTA-Ahx-STp(5-18) PK and Biodistribution

This study is a pharmacokinetics/biodistribution study in naïve SCID mice. This study assays the effects of renal protectants (e.g., Clinisol, Lysine, Lysine/arginine, Gelofusine and/or amifostine) on ²¹³Bi-DOTA-Ahx-STp(5-18) pharmacokinetics and biodistribution.

### Dosing Regimen: ²¹³Bi-DOTA-Ahx-STp(5-18)

1. ²¹³Bi-DOTA-Ahx-STp(5-18) control with no renal protectant (IV tail vein) (n=3×4).
2. ²¹³Bi-DOTA-Ahx-STp(5-18) coadministered with Lysine/Arginine (IV tail vein) (n=3×4).
3. ²¹³Bi-DOTA-Ahx-STp(5-18) coadministered with amifostine (IV tail vein) (n=3×4).
4. ²¹³Bi-DOTA-Ahx-STp(5-18) coadministered with Lysine/Arginine and amifostine (IV tail vein) (n=3×4).

Other renal protectants, e.g., Clinisol, Lysine, and Gelfusine, may be tested alone or in combination using a similar dosing regimen as described above.

### Data Collection:

1. Tissue is resected, weighed, and counted at 3 time points for each dosing regimen.
   i. 15 min (n=4)
   ii. 1 h (n=4)
   iii. 3 h (n=4)
2. At time of sacrifice, tissues (blood, heart, kidney, liver, lungs, spleen, pancreas, bladder, small intestine, large intestine, stomach, and bone) are resected, weighed, and counted.

### Example 16

### Tolerability of ²¹³Bi-DOTA-Ahx-STp(5-18) in HEK-293 GCC Tumor Bearing Mice

This study assays maximum tolerated dose (MTD) of ²¹³Bi-DOTA-Ahx-STp(5-18) plus and minus renal protectant (e.g., Clinisol, Lysine, Lysine/arginine, Gelofusine and/or amifostine). The MTD is determined as the highest administered activity that allows 100% survival with no significant body weight loss (>20%). Tumor, blood, heart, kidney, liver, spleen, lung, femur, and small intestine will be resected and fixed for H&E staining to understand the radiation exposure levels and their affect on normal and tumor tissue. Blood urea nitrogen and serum creatinine levels will also be measured to look for potential radiation damage to the kidney.

### Dosing Regimen:

1. Negative control: Unlabeled STp(5-18) (IV tail vein) peptide control (n=4).

2. 10 kBq ²¹³Bi-DOTA-Ahx-STp(5-18)+renal protectant (IV tail vein) (n=4).

3. 20 kBq ²¹³Bi-DOTA-Ahx-STp(5-18)+renal protectant (IV tail vein) (n=4).

4. 40 kBq ²¹³Bi-DOTA-Ahx-STp(5-18)+renal protectant (IV tail vein) (n=4).

5. 60 kBq ²¹³Bi-DOTA-Ahx-STp(5-18)+renal protectant (IV tail vein) (n=4).

6. 120 kBq ²¹³Bi-DOTA-Ahx-STp(5-18)+renal protectant (IV tail vein) (n=4).

7. 60 kBq ²¹³Bi-DOTA-Ahx-STp(5-18) (IV) (n=4).

8. 120 kBq ²¹³Bi-DOTA-Ahx-STp(5-18) (IV) (n=4).

Renal protectants such as Clinisol, lysine/arginine, Lysine, amofistine and Gelofusine, may be tested alone or in combination using a similar dosing regimen as described above.

### Data Collection:

1. Tumor volume and Mouse weight measurements 3× per week for 21 days.
2. At time of sacrifice (animals will be sacrificed if tumor size >2000 mm³ or body weight loss >20%), resect tissues (tumor, blood, heart, kidney, liver, spleen, lung, femur, small intestine), weigh, and formalin fix for IHC (H&E).
   a. Fix kidney and tumor for IHC analysis.
3. Measurement of blood urea nitrogen and serum creatinine levels.

### Example 17

### Efficacy Study of ²¹³Bi-DOTA-Ahx-STp(5-18) in HEK-293 GCC#2 Tumor Bearing Mice

This study assays the efficacy of ²¹³Bi-DOTA-Ahx-STp(5-18) at MTD using different dosing schedules. At the end of the study, tissue will be resected to understand the radiation exposure levels and their affect on normal and tumor tissue. Blood urea nitrogen and serum creatinine levels will also be measured to look for potential radiation damage to the kidney.

### Dosing Regimen:

1. Negative control: Unlabeled STp (5-18)+renal protectant, (IV tail vein) peptide control (n=8).
2. MTD uCi ²¹³Bi-DOTA-Ahx-STp(5-18)+renal protectant, (IV tail vein) peptide control (n=8).
3. ½ MTD uCi ²¹³Bi-DOTA-Ahx-STp(5-18)+renal protectant once a week for 2 weeks (IV tail vein) (n=8).
4. ⅓ MTD uCi ²¹³Bi-DOTA-Ahx-STp(5-18)+renal protectant once a week for 3 weeks (IV tail vein) (n=8).

### Data Collection:

1. Tumor caliper measurements and body weights two times a week (with constant monitoring for any adverse affects) until animals are sacrificed (animals will be sacrificed if tumor size >2000 mm³ or body weight loss >20%).
2. At time of sacrifice, tissues (tumor, heart, kidney, liver, spleen, lung) are collected and fixed in formalin for possible future IHC analysis.

### Example 18

### Assessing the Pharmacokinetics and Ex Vivo Biodistribution of Actinium-225 Labeled STp(5-18) Peptide in Naïve CB-17 SCID Mice

This study assays the pharmacokinetics and ex vivo biodistribution of Actinium-225 labeled STp(5-18) peptide, along with the daughter nuclides, in naïve CB-17 SCID mice.

1 mCi 225Ac was obtained from Oak Ridge National Laboratory in the form of solid actinium nitrate, carrier-free. 50 ug DOTA-Ahx-STp(5-18) was dissolved in 500 uL of 0.2 M sodium acetate buffer containing 20% ethanol at pH 5.6. 1 mCi 225Ac nitrate was reconstituted with 200 uL of 0.2 M HCl. 20 uL (100 uCi) of 225Ac was transferred to the reaction vial and the vial was heated at 100 C for 10 min. Radiochemical purity was >96% by iTLC with 50 mM sodium citrate pH 5.5 mobile phase.

The ²²⁵AC-DOTA-Ahx-STp(5-18) radiolabeled peptide was purified using a SepPak C18 cartridge and eluted with 500 ul of 1:1 isotonic saline/absolute ethanol

The ex vivo biodistribution of ²²⁵Ac-DOTA-Ahx-STp(5-18) was observed in 12 naive CB-17 SCID female mice (Taconic Farms, Inc.). All naïve mice were randomized into n=4×3 groups. All mice were administered 100 uL of formulated 225Ac-DOTA-Ahx-STp(5-18), IV, within 45 minutes of purification (100 uL=444598 CPM counted on the 225Ac window of the gamma counter). A subset of animals from each group was sacrificed at each of the following time points post injection: 0.5, 1, 4, and 24 hours. The following tissues were resected for weight and gamma counting from each carcass: blood, heart, kidney, liver, lungs, spleen, small intestine, large intestine, stomach. The ²²⁵Ac content was determined at least 4 h after collection, to allow for ^{213B}i/²²⁵Ac equilibrium. To analyze daughter activities, the counter was calibrated and windows were set to collect gamma events from ²²¹Fr (g, 218 keV) for actinium quantitation and ²¹³Bi (g, 440 keV). Samples were counted within 20-50 min post-harvest and again after 213Bi/225Ac equilibrium had been reached (>4 h).

### Study Design Summary

| | | | | Radio- | |
|---|---|---|---|---|---|
| | | | | activity | |
| Study | No. | | Mass Dose | Dose | |
| & | Female | | (μg/ | (μCi/ | Time Points |
| Route | Animals | Tracer | animal) | animal) | (h) |
| 1 IV | 12 | ²²⁵Ac- DOTA- | 2 μg | 3.7 μCi | 0.5 (n = 3) |
| | | Ahx-STp(5-18) | | | 1 (n = 3) |
| | | | | | 4 (n = 3) |
| | | | | | 24 (n = 3) |

The gamma count runs were acquired according to the procedure described by Song et al. Specifically, samples were measured at three times immediately following sacrifice (˜30, 45, 60 minutes) in ²²¹Fr and ²¹³Bi windows and again at equilibrium (24 h post-sacrifice).

### Actinium Analysis

Data, including weight, time point, injection times, sacrifice times, subject ID, tissue, and CPM were read in automatically from the equilibrium ²²⁵Ac spreadsheet. The measured standard had an identical amount of radioactivity to the injected dose and so the measured standard CPM was used as the injected dose. Percent injected dose per gram was computed as

$$\% ID / {g}_{tissue} = \frac{{CPM}_{tissue}}{{CPM}_{standard}} * \frac{100}{{wght}_{tissue}}$$

Percent injected dose per gram values were written into a spreadsheet and plotted against time point.

### Francium-221/Bismuth-213 Analysis

Data, including weight, time point, injection times, sacrifice times, subject ID, tissue, and CPM were read in automatically from the ²¹³Bi and ²²¹Fr spreadsheets, including CPM measurements for three time points immediately following sacrifice (˜30, 45, 60 minutes post-injection). CPM estimates from the three time points immediately following sacrifice were fit to a single-exponential using an ordinary least squares fit to the log of the CPM data. Exact times post-sacrifice are computed for each CPM value using the injection time and time point information as reference. The estimated CPM at time of sacrifice (CPM_0) was estimated based on the fits. This CPM_0 was compared to the measured CPM at equilibrium decay-corrected to time-of-sacrifice (CPM_eq) both in terms of absolute difference and ratio. A difference (CPM_0−CPM_eq) greater than zero indicates accumulation of the ²²¹Fr or ²¹³Bi in the tissue.

### Summary of Results

Using the methods described under this study, STp(5-18) peptide was successfully labeled with ²²⁵Ac at a specific activity of 4.2 mCi/nmol.

The half-life of ²²⁵AC-DOTA-Ahx-STp(5-18) in blood was 318 min. As a comparison, ⁶⁸Ga-DOTA-Ahx-STp(5-18) had a half-life of 26 min.

There was high uptake of ²²⁵Ac-DOTA-Ahx-STp(5-18) in the kidney, as expected, and no substantial accumulation in the other tissues measured. Likewise, there was substantially no accumulation of the ²²¹Fr or ²¹³Bi daughter decay products in any of the tissues except kidney.

### Example 19

### Tolerability Study of Actinium-225 Labeled STp(5-18) Peptide in HEK-293 GCC Tumor Bearing Mice

This study assays the maximum tolerated dose (MTD) of ²²⁵Ac-DOTA-Ahx-STp(5-18) plus and minus renal protectant (e.g., Clinisol, Lysine, Lysine/arginine, Gelofusine and/or amifostine). The MTD is determined as the highest administered activity that allows 100% survival with no significant body weight loss (>20%). Tumor, blood, heart, kidney, liver, spleen, lung, femur, and small intestine will be resected and fixed for H&E staining to understand the radiation exposure levels and their affect on normal and tumor tissue. Blood urea nitrogen and serum creatinine levels will also be measured to look for potential radiation damage to the kidney.

### Dosing Regimen:

1. Negative control: Unlabeled STp(5-18) (IV tail vein) peptide control (n=4).

2. 10 kBq ²²⁵Ac-DOTA-Ahx-STp(5-18) (IV tail vein) (n=4).

3. 20 kBq ²²⁵Ac-DOTA-Ahx-STp(5-18) (IV tail vein) (n=4).

4. 40 kBq ²²⁵Ac-DOTA-Ahx-STp(5-18) (IV tail vein) (n=4).

5. 60 kBq ²²⁵Ac-DOTA-Ahx-STp(5-18) (IV tail vein) (n=4).

6. 100 kBq ²²⁵Ac-DOTA-Ahx-STp(5-18) (IV tail vein) (n=4).

7. 40 kBq ²²⁵Ac-DOTA-Ahx-STp(5-18)+lysine/arginine (IV) pretreatment (n=4).

8. 40 kBq ²²⁵Ac-DOTA-Ahx-STp(5-18)+100 mg/kg amifostine (IV) pretreatment (n=4).

Other renal protectants, e.g., Clinisol, Lysine, and Gelofusine, may be tested alone or in combination using a similar dosing regimen as described above.

### Data Collection:

1. Tumor volume and Mouse weight measurements are taken 3× per week for 21 days.
2. At time of sacrifice (animals will be sacrificed if tumor size >2000 mm³ or body weight loss >20%), resect tissues (tumor, blood, heart, kidney, liver, spleen, lung, femur, small intestine), weigh, and formalin fix for IHC (H&E).
   a. Fix kidney and tumor for IHC analysis.
3. Measurement of blood urea nitrogen and serum creatinine levels.

### Example 20

### ²²⁵AC-DOTA-Ahx-STp(5-18) Efficacy Study in HEK-293 GCC#2 Tumor Bearing Mice

This study evaluates the efficacy of ²²⁵AC-DOTA-Ahx-STp(5-18) at MTD using different dosing schedules, and with or without renal protectant (e.g., Clinisol, Lysine, Lysine/arginine, Gelfusine and/or amifostine). At the end of the study tissue will be resected to understand the radiation exposure levels and their affect on normal and tumor tissue. Blood urea nitrogen and serum creatinine levels will also be measured to look for potential radiation damage to the kidney.

### Dosing Regimen:

1. Negative control: Unlabeled STp(5-18) (IV tail vein) peptide control (n=8).
2. MTD nCi ²²⁵AC-DOTA-Ahx-STp(5-18) single dose (IV tail vein) (n=8).
3. MTD nCi ²²⁵AC-DOTA-Ahx-STp(5-18) single dose with renal protectant (IV tail vein) (n=8).
4. ½ MTD×2 nCi ²²⁵AC-DOTA-Ahx-STp(5-18) once a week for 2 weeks (IV tail vein) (n=8).
5. ⅓ MTD nCi ²²⁵Ac-DOTA-Ahx-STp(5-18) once a week for 3 weeks (IV tail vein) (n=8).

### Data Collection:

1. Tumor volume and Mouse weight measurements will be taken 3× per week for 21 days.
2. At time of sacrifice (animals will be sacrificed if tumor size >2000 mm³ or body weight loss >20%), resect tissues (tumor, blood, heart, kidney, liver, spleen, lung, femur, small intestine), weigh, and formalin fix for IHC (H&E).
3. Measurement of blood urea nitrogen and serum creatinine levels.

What is claimed is:

1. A compound comprising:
(a) a peptide comprising an amino acid sequence of SEQ ID NO: 1, wherein the peptide has an amino terminus and has a free carboxy-terminus,
(b) a chelating moiety capable of binding a radioactive atom, wherein the chelating moiety comprises a macrocycle, e.g., a macrocycle comprising an O and/or a N, DOTA, NOTA, one or more amines, one or more ethers, one or more carboxylic acids, EDTA, DTPA, TETA, DO3A, PCTA, or desferrioxamine, and
(c) a linker moiety that covalently attaches the amino-terminus of the amino acid sequence of the peptide to the chelating moiety,
wherein the compound is capable of binding to guanylyl cyclase C (GCC).

2. The compound of claim 1, wherein the peptide comprises a GCC-binding portion of the amino acid sequence of SEQ ID NO: 2.

3. The compound of claim 1 or 2, wherein the peptide comprises the amino acid sequence of SEQ ID NO: 3.

4. The compound of claim 3, wherein disulfide bonds connect Cys5 to Cys10, Cys6 to Cys14, and Cys9 to Cys17, wherein the amino acids are numbered according to their position in native E. coli enterotoxin.

5. The compound of claim 1, wherein the peptide consists of the amino acid sequence of SEQ ID NO: 2.

6. The compound of claim 1, wherein the peptide consists of the amino acid sequence of SEQ ID NO:1.

7. The compound of any of the preceding claims, wherein the chelating moiety comprises a macrocycle.

8. The compound of any of the preceding claims, wherein the chelating moiety is a macrocycle.

9. The compound of any of claims 1-7, wherein the chelating moiety comprises DOTA or NOTA.

10. The compound of any of claims 1-6, wherein the chelating moiety is DOTA or NOTA.

11. The compound of any of the preceding claims, wherein the linker moiety comprises an aminopentyl, aminohexyl, or aminoheptyl group.

12. The compound of any of claims 1-6, wherein the linker moiety is an aminopentyl, aminohexyl, or aminoheptyl group.

13. The compound of any of claims 1-11, wherein the linker moiety comprises an alkylene, e.g., C₁-C₁₀ alkylene such as C₁, C₂, C₃, C₄, C₅, C₆, C₇, C₈, C₉, or C₁₀ alkylene.

14. The compound of any of claims 1-11 and 13, wherein the linker moiety comprises O or S.

15. The compound of any of claims 1-11, 13, and 14, wherein the linker moiety comprises one or more of urea, thiourea, and benzyl.

16. The compound of claim 1, wherein the compound is a 2,2′,2″,2′″-((S)-2-(4-(3-(6-(((3S,6R,9S,15R,20R,23S,26S,29R,32R,37R,40S,45aS)-40-(2-amino-2-oxoethyl)-15-(((S)-1-carboxy-2-(4-hydroxyphenyl)ethyl)carbamoyl)-26-(2-carboxyethyl)-23-isobutyl-3,9-dimethyl-1,4,7,10,13,22,25,28,31,38,41,47-dodecaoxotetracontahydro-1H-37,20-(epiminomethano)-6,29-(methanodithiomethano)pyrrolo[2,1-s][1,2,27,28,5,8,11,14,17,20,23,32,35,38,41]tetrathiaundecaazacyclotritetracontin-32-yl)amino)-6-oxohexyl)thioureido)benzyl)-1,4,7,10-tetraazacyclododecane-1,4,7,10-tetrayl)tetraacetic acid.

17. The compound of claim 1, wherein the compound has the structure of Formula (VI):

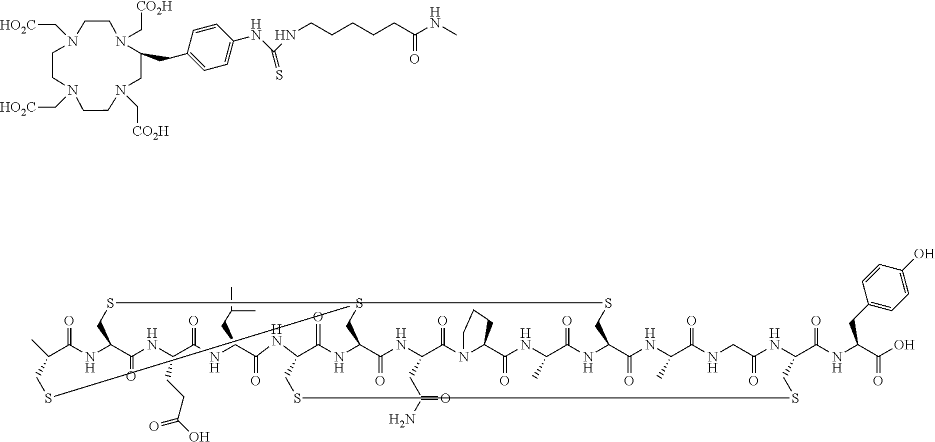 [SMILES: CNC(=O)CCCCCNC(=S)Nc1ccc(C[C@H]2CN(CC(=O)O)CCN(CC(=O)O)CCN(CC(=O)O)CCN2CC(=O)O)cc1 ; CC(C)C[C@@H]1NC(=O)[C@H](CCC(=O)O)NC(=O)[C@@H]2CSSC[C@@H]3NC(=O)[C@H](C)NC(=O)[C@@H]4CCCN4C(=O)[C@H](CC(N)=O)NC(=O)[C@H](CSSC[C@H](C)C(=O)N2)NC(=O)[C@H](CSSC[C@@H](C(=O)N[C@@H](Cc2ccc(O)cc2)C(=O)O)NC(=O)CNC(=O)[C@H](C)NC3=O)NC1=O]

18. The compound of any of the above claims, further comprising a radioactive atom bound to the chelating moiety.

19. The compound of claim 18, wherein the radioactive atom is a positron-emitter.

20. The compound of claim 19, wherein the radioactive atom is Gallium-68.

21. The compound of claim 18, wherein the radioactive atom is an alpha particle emitter, e.g., ²¹³Bi or ²²⁵Ac.

22. The compound of claim 21, wherein the radioactive atom is a beta particle emitter, e.g., ⁹⁰Y or ¹⁷⁷Lu.

23. The compound of claim 18, wherein the radioactive atom is a gamma-emitter.

24. The compound of claim 23, wherein the radioactive atom is ¹¹¹In.

25. The compound of any of claims 18-24, which has a specific activity of about 20 to 40 MBq/nmol, e.g., 25-33 MBq/nmol.

26. The compound of claim 25, which has a specific activity of about 29 MBq/nmol.

27. The compound of claim 18, wherein the compound has the structure of Formula (VIa):

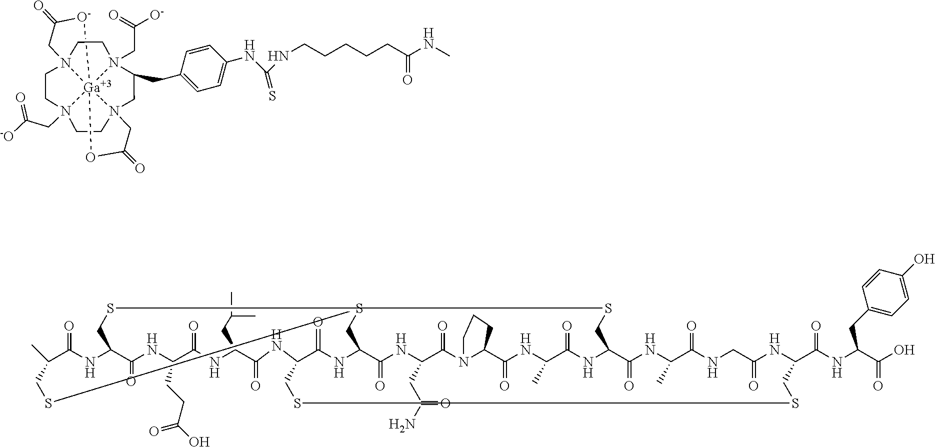

28. A composition comprising a plurality of compounds according to any one of claims 1-17, wherein at least one compound of the plurality is bound to a radioactive atom and at least one compound of the plurality is not bound to a radioactive atom.

29. The composition of claim 28, wherein the radioactive atom is a positron-emitting atom.

30. The composition of claim 29, wherein the positron-emitting atom is Gallium-68.

31. The composition of claim 28, wherein at least one of the compounds of the plurality has a structure of Formula (VIa), and at least one of the compounds of the plurality has a structure of Formula (VI).

32. The composition of any of claims 28-31, which has a ratio of about 1:100 to 1:10,000 of Gallium-68-bound compounds to unbound compounds.

33. The composition of claim 32, which has a ratio of about 1:1,000 to 1:2,000 of Gallium-68-bound molecules to unbound molecules.

34. The composition of claim 33, which has a ratio of about 1:1,500 of Gallium-68-bound molecules to unbound molecules.

35. A compound comprising:
(a) a peptide comprising an amino acid sequence of SEQ ID NO: 1, wherein the peptide has an amino terminus and has a free carboxy-terminus,
(b) a chelating moiety capable of binding a positron-emitting atom,
(c) a linker moiety that covalently attaches the amino-terminus of the amino acid sequence of the peptide to the chelating moiety, and
(d) a positron-emitting atom, e.g., gallium-68,
wherein the compound is capable of binding to guanylyl cyclase C (GCC).

36. The compound of claim 35, wherein the peptide comprises a GCC-binding portion of the amino acid sequence of SEQ ID NO: 2.

37. The compound of claim 35 or 36, wherein the peptide comprises the amino acid sequence of SEQ ID NO: 3.

38. The compound of claim 37, wherein disulfide bonds connect Cys5 to Cys10, Cys6 to Cys14, and Cys9 to Cys17, wherein the amino acids are numbered according to their position in native E. coli enterotoxin.

39. The compound of claim 35, wherein the peptide consists of the amino acid sequence of SEQ ID NO: 2.

40. The compound of claim 35, wherein the peptide consists of the amino acid sequence of SEQ ID NO:1.

41. The compound of any of claims 35-40, wherein the chelating moiety comprises a macrocycle.

42. The compound of any of claims 35-41, wherein the chelating moiety is a macrocycle.

43. The compound of any of claims 35-41, wherein the chelating moiety comprises DOTA or NOTA.

44. The compound of any of claims 35-40, wherein the chelating moiety is DOTA or NOTA.

45. The compound of any of claims 35-41, wherein the chelating moiety comprises a macrocycle, e.g., a macrocycle comprising an O and/or a N, DOTA, NOTA, one or more amines, one or more ethers, one or more carboxylic acids, EDTA, DTPA, TETA, DO3A, PCTA, or desferrioxamine.

46. The compound of any of claims 35-45, wherein the linker moiety comprises an aminopentyl, aminohexyl, or aminoheptyl group.

47. The compound of any of claims 35-40, wherein the linker moiety is an aminopentyl, aminohexyl, or aminoheptyl group.

48. The compound of any of claims 35-46, wherein the linker moiety comprises an alkylene, e.g., C₁-C₁₀ alkylene such as C₁, C₂, C₃, C₄, C₅, C₆, C₇, C₈, C₉, or C₁₀ alkylene.

49. The compound of any of claims 35-46 and 48, wherein the linker moiety comprises O or S.

50. The compound of any of claims 35-46, 48, and 49, wherein the linker moiety comprises urea or thiourea.

51. The compound of any of claims 35-46 and 48-50, wherein the linker moiety comprises a benzyl group.

52. The compound of claim 1, wherein the compound is a 2,2′,2″,2′″-((S)-2-(4-(3-(6-(((3S,6R,9S,15R,20R,23S,26S,29R,32R,37R,40S,45aS)-40-(2-amino-2-oxoethyl)-15-(((S)-1-carboxy-2-(4-hydroxyphenyl)ethyl)carbamoyl)-26-(2-carboxyethyl)-23-isobutyl-3,9-dimethyl-1,4,7,10,13,22,25,28,31,38,41,47-dodecaoxotetracontahydro-1H-37,20-(epiminomethano)-6,29-(methanodithiomethano)pyrrolo[2,1-s][1,2,27,28,5,8,11,14,17,20,23,32,35,38,41]tetrathiaundecaazacyclotritetracontin-32-yl)amino)-6-oxohexyl)thioureido)benzyl)-1,4,7,10-tetraazacyclododecane-1,4,7,10-tetrayl)tetraacetic acid.

53. The compound of any of claims 35-52, wherein the radioactive atom is Gallium-68.

54. The compound of any of claims 35-53, which has a specific activity of about 20 to 40 MBq/nmol, e.g., 25-33 MBq/nmol.

55. The compound of claim 54, which has a specific activity of about 29 MBq/nmol.

56. The compound of claim 35, wherein the compound has the structure of Formula (VIa):

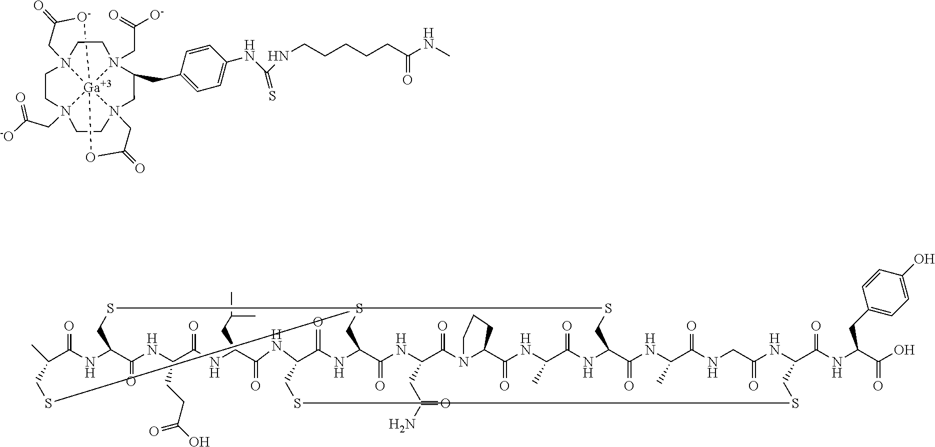

57. A composition comprising:
(a) a compound according to any one of claims 1-17,
(b) ethanol,
(c) sodium acetate, and
(d) water,
wherein the composition has a pH of about 3-6.

58. The composition of claim 57, wherein the compound is the compound of claim 6b.

59. The composition of claim 57 or 58, wherein the ethanol is present at about 10-25%.

60. The composition of claim 59, wherein the ethanol is present at about 20-25%.

61. The composition of any of claims 57-60, which has a pH of about 3.8.

62. The composition of any of claims 57-61, further comprising one or more of sucrose, glacial acetic acid, polysorbate 80, and Pluronic F-68.

63. The composition of any of claims 57-62, wherein the compound has a concentration of about 0.01-0.04 μg/ml.

64. A composition comprising a compound according to any one of claims 1-17, in aqueous solution, wherein the compound has a concentration of about 0.01-0.04 μg/ml.

65. The composition of claim 64, wherein the concentration is about 0.027 μg/ml.

66. A container comprising the composition of any of claims 57-65.

67. The container of claim 66, wherein the composition has a volume of about 2 ml.

68. A kit comprising the container of claim 66 or 67, and a cassette suitable for contacting the compound with radioactive material.

69. The kit of claim 68, further comprising written instructions for radiolabeling the compound with the radioactive material, written instructions for a method of administering the compound to a subject, or both.

70. A composition comprising:
(a) a compound according to any one of claims 18-56,
(b) ethanol,
(c) sodium chloride, and
(d) water.

71. The composition of claim 70, wherein the ethanol is present at about 5-25%.

72. The composition of claim 70, wherein the ethanol is present at about 5-20%.

73. The composition of claim 70, wherein the ethanol is present at about 5-15%.

74. The composition of claim 70, wherein the ethanol is present at about 8%.

75. The composition of any of claims 70-74, wherein the sodium chloride is present at about 0.3-1.4%.

76. The composition of any of claims 70-74, wherein the sodium chloride is present at about 0.4-1.0%.

77. The composition of any of claims 70-74, wherein the sodium chloride is present at about 0.7-1.0%.

78. The composition of any of claims 70-74, wherein the sodium chloride is present at about 0.8%-0.9%.

79. The composition of any of claims 70-78, which has a volume of about 8-11 ml.

80. The composition of any of claims 70-78, which has a volume of about 9-10 ml.

81. The composition of claim 80, which has a volume of about 9.5 ml.

82. The composition of any of claims 70-81, which comprises about 5.1-7.6 mCi of radioactivity.

83. The composition of any of claims 70-81, which comprises about ≧12.73 mCi of radioactivity.

84. The composition of any of claims 70-83, which comprises about 20-70 μg of the compound.

85. The composition of claim 84, which comprises about 55.0 μg of the compound.

86. A composition comprising a compound according to any one of claim 18-27 or 35-56 and a pharmaceutically acceptable excipient.

87. A container comprising the composition of any of claims 57-86.

88. The container of claim 87, which is a vial.

89. A method of assaying for a GCC-expressing cell in a subject, comprising:
(a) administering, e.g., intravenously, the compound of any of claim 18-20, 23-27, or 35-56 or the composition of any of claims 70-86 to the subject, and
(b) visualizing the distribution of radioactivity in the subject.

90. The method of claim 89, wherein the compound is a compound of claim 17 bound to Gallium-68.

91. The method of claim 89 or 90, wherein the radioactivity is visualized by positron emission tomography (PET).

92. The method of any of claims 89-91, wherein the compound administered has about 4.0 to 6.0 (±10%) mCi.

93. The method of any of claims 89-92, wherein the compound is administered at a dose of about 40-46 ug.

94. The method of claim 93, wherein the compound is administered at a dose of about 43.4 ug.

95. The method of any of claims 89-94, wherein the subject is a human.

96. The method of any of claims 89-95, wherein the subject is suffering from a disorder associated with GCC expression.

97. The method of claim 96, wherein the disorder is cancer.

98. The method of claim 97, wherein the disorder is selected from solid tumor, a soft tissue tumor, a metastatic lesion, a sarcoma, an adenocarcinoma, or a carcinoma.

99. The method of claim 97 or 98, wherein the cancer is early or late stage cancer, or cancer of any of stages 0, 1, IIA, IIB, IIIA, IIIB, IIIC, and IV.

100. The method of any of claims 97-99, wherein the disorder is selected from colorectal cancer, gastric cancer, esophageal cancer, cancer of the gastroesophageal junction, pancreatic cancer, lung cancer, small intestine cancer, leiomyosarcoma, rhabdomyosarcoma, and a neuroendocrine tumor, or any metastases thereof.

101. The method of claim 100, wherein the colorectal cancer is a colorectal adenocarcinoma, colorectal leiomyosarcoma, colorectal lymphoma, colorectal melanoma, or colorectal neuroendocrine tumor.

102. The method of claim 100, wherein the gastric cancer is a gastric adenocarcinoma, gastric sarcoma, or gastric lymphoma.

103. The method of claim 100, wherein the esophageal cancer is esophageal squamous cell carcinoma, or esophageal adenocarcinoma.

104. The method of claim 100, wherein the lung cancer (e.g., non-small cell or small cell lung cancer) is a squamous cell carcinoma or adenocarcinoma.

105. The method of claim 100, wherein the neuroendocrine tumor is a gastrointestinal or a bronchopulmonary neuroendocrine tumor.

106. The method of any of claims 89-105, further comprising administering an agent that ameliorates bladder toxicity associated with the administered compound.

107. The method of claim 106, wherein the agent that ameliorates bladder toxicity increases urinary excretion.

108. The method of claim 106, wherein the agent that ameliorates bladder toxicity is saline or water.

109. The method of any of claims 89-108, further comprising performing partial volume correction on an image representing the distribution of radioactivity in the patient.

110. A method of determining sensitivity of cancer cells to a GCC-targeted therapeutic agent, comprising assaying for a GCC-expressing cell in the subject according to the method of any of claims 89-109, wherein binding of the radiolabel to the cancer cell indicates sensitivity to the GCC-targeted therapeutic agent.

111. A method of evaluating whether a subject is a potential candidate for a GCC-targeted therapy, comprising assaying for a GCC-expressing cell in the subject according to the method of any of claims 89-109, wherein binding of the radiolabel to the cancer cell indicates that the subject is a potential candidate for a GCC-targeted therapy

112. Use of the compound of any of claim 18-20, 23-27, or 35-56 or the composition of any of claims 70-86 for detecting a GCC-expressing cell in a subject.

113. Use of the compound of any of claim 18-20, 23-27, or 35-56 or the composition of any of claims 70-86 in the manufacture of a composition for detecting a GCC-expressing cell in a subject.

114. A method of treating a subject having a disorder characterized by one or more GCC-expressing cells, comprising:
(a) assaying for a GCC-expressing cell in the subject according to the method of any of claims 89-109, and
(b) if the subject has one or more cells that express GCC, administering a GCC-targeted therapeutic agent.

115. The method of claim 114, wherein the GCC-targeted therapeutic agent comprises an anti-GCC antibody molecule selected from:
(a) an anti-GCC antibody molecule comprising three heavy chain complementarity determining regions (CDR1, CDR2, and CDR3) comprising amino acid sequences provided in Table 3; and three light chain complementarity determining regions (CDR1, CDR2, and CDR3) comprising amino acid sequences provided in Table 3,
(b) an anti-GCC antibody molecule that is capable of competing for binding with an anti-GCC antibody comprising three heavy chain complementarity determining regions (CDR1, CDR2, and CDR3) comprising amino acid sequences provided in Table 3 and three light chain complementarity determining regions (CDR1, CDR2, and CDR3) comprising amino acid sequences provided in Table 3, and
(c) an anti-GCC antibody molecule that is capable of binding to the same epitope as an anti-GCC antibody comprising three heavy chain complementarity determining regions (CDR1, CDR2, and CDR3) comprising amino acid sequences provided in Table 3 and three light chain complementarity determining regions (CDR1, CDR2, and CDR3) comprising amino acid sequences provided in Table 3.

116. The method of claim 114, wherein the GCC-targeted therapeutic agent comprises an anti-GCC antibody molecule selected from:
(a) an anti-GCC antibody molecule comprising a heavy chain CDR1 of SEQ ID NO: 5, a heavy chain CDR2 of SEQ ID NO: 6, a heavy chain CDR3 of SEQ ID NO: 7, a light chain CDR1 of SEQ ID NO: 8, a light chain CDR2 of SEQ ID NO: 9, and a light chain CDR3 of SEQ ID NO: 10,
(b) an anti-GCC antibody molecule that is capable of competing for binding with an anti-GCC antibody comprising three heavy chain complementarity determining regions (CDR1, CDR2, and CDR3) comprising a heavy chain CDR1 of SEQ ID NO: 5, a heavy chain CDR2 of SEQ ID NO: 6, a heavy chain CDR3 of SEQ ID NO: 7, a light chain CDR1 of SEQ ID NO: 8, a light chain CDR2 of SEQ ID NO: 9, and a light chain CDR3 of SEQ ID NO: 10, and
(c) an anti-GCC antibody molecule that is capable of binding to the same epitope as an anti-GCC antibody comprising a heavy chain CDR1 of SEQ ID NO: 5, a heavy chain CDR2 of SEQ ID NO: 6, a heavy chain CDR3 of SEQ ID NO: 7, a light chain CDR1 of SEQ ID NO: 8, a light chain CDR2 of SEQ ID NO: 9, and a light chain CDR3 of SEQ ID NO: 10.

117. The method of any of claims 114-116, wherein the GCC-targeted therapeutic comprises a heavy chain that comprises an amino acid sequence of SEQ ID NO: 11 and a light chain that comprises an amino acid sequence of SEQ ID NO: 12.

118. The method of any of claims 114-117, wherein the GCC-targeted therapeutic agent is an antibody-drug conjugate.

119. The method of claim 118, wherein the antibody molecule is conjugated to monomethyl auristatin E (MMAE).

120. The method of claim 118, wherein the antibody-drug conjugate comprises a protease-cleavable linker.

121. The method of claim 118, wherein the antibody-drug conjugate comprises the following Formula I-5: 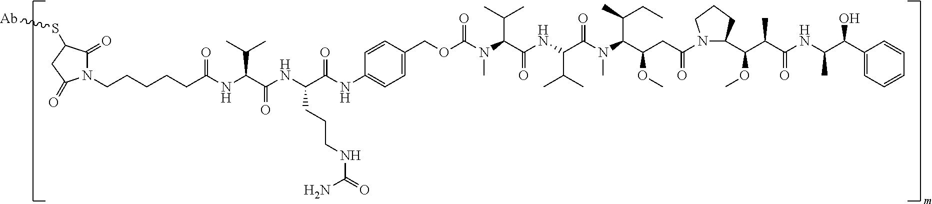
or a pharmaceutically acceptable salt thereof, wherein:
Ab is an anti-GCC antibody molecule, or antigen binding fragment thereof, and m is an integer from 1-8 (e.g., 3-5, 4).

122. The method of claim 114, wherein the GCC-targeted therapeutic agent comprises:
the following Formula I-5: 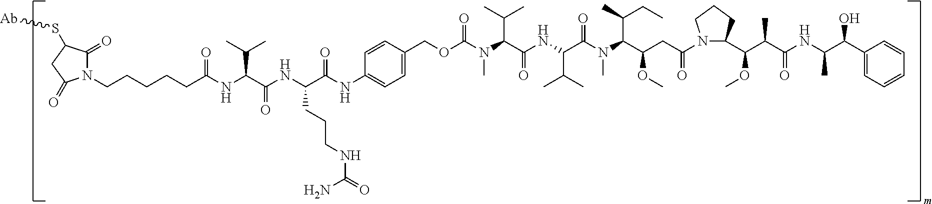
or a pharmaceutically acceptable salt thereof, wherein:
Ab is an anti-GCC antibody molecule, or antigen binding fragment thereof, that comprises a heavy chain CDR1 of SEQ ID NO: 5, a heavy chain CDR2 of SEQ ID NO: 6, a heavy chain CDR3 of SEQ ID NO: 7, a light chain CDR1 of SEQ ID NO: 8, a light chain CDR2 of SEQ ID NO: 9, and a light chain CDR3 of SEQ ID NO: 10, and
m is an integer from 1-8 (e.g., 3-5, 4).

123. The method of claim 114, wherein the GCC-targeted therapeutic agent is MLN0264.

124. The method of any of claims 114-123, wherein the disorder is cancer.

125. The method of claim 124, wherein the disorder is gastric cancer, pancreatic cancer, esophageal cancer, cancer of the gastroesophageal junction, or colorectal cancer.

126. A method of treating a disorder characterized by one or more GCC-expressing cells, comprising administering a therapeutically effective amount of a compound according to any one of claim 21 or 22 to a patient in need thereof.

127. The method of claim 126, wherein the disorder is cancer.

128. The method of claim 127, wherein the disorder is selected from solid tumor, a soft tissue tumor, a metastatic lesion, a sarcoma, an adenocarcinoma, or a carcinoma.

129. The method of claim 127 or 128, wherein the cancer is early or late stage cancer, or cancer of any of stages 0, 1, IIA, IIB, IIIA, IIIB, IIIC, and IV.

130. The method of any of claims 127-129, wherein the disorder is selected from colorectal cancer, gastric cancer, esophageal cancer, cancer of the gastroesophageal junction, small intestine cancer, pancreatic cancer, lung cancer, leiomyosarcoma, rhabdomyosarcoma, and a neuroendocrine tumor, or any metastases thereof.

131. The method of claim 130, wherein the colorectal cancer is a colorectal adenocarcinoma, colorectal leiomyosarcoma, colorectal lymphoma, colorectal melanoma, or colorectal neuroendocrine tumor.

132. The method of claim 130, wherein the gastric cancer is a gastric adenocarcinoma, gastric sarcoma, or gastric lymphoma.

133. The method of claim 130, wherein the esophageal cancer is esophageal squamous cell carcinoma or esophageal adenocarcinoma.

134. The method of claim 130, wherein the lung cancer is a squamous cell carcinoma or adenocarcinoma.

135. The method of claim 130, wherein the neuroendocrine tumor is a gastrointestinal or a bronchopulmonary neuroendocrine tumor.

136. The method of any of claims 126-135, further comprising administering an additional form of therapy to the patient.

137. The method of claim 136, wherein the additional form of therapy is radiation therapy.

138. The method of claim 136, wherein the additional form of therapy is a second therapeutic molecule.

139. The method of claim 138, wherein the second therapeutic molecule is a DNA-damaging agent.

140. The method of claim 139, wherein the DNA-damaging agent is selected from a topoisomerase I inhibitor, a topoisomerase II inhibitor, an alkylating agent, an alkylating-like agent, an athracylcine, a DNA intercalator, a DNA minor groove alkylating agent, and an antimetabolite.

141. The method of claim 140, wherein the DNA damaging agent is a topoisomerase I inhibitor selected from irinotecan, topotecan, and camptothecin.

142. The method of claim 140, wherein the DNA damaging agent is an alkylating-like agent selected from cisplatin, oxaliplatin, carboplatin, nedaplatin, satraplatin and triplatin.

143. The method of claim 140, wherein the DNA damaging agent is an antimetabolite selected from fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, and pemetrexed.

144. The method of any of claims 126-143, wherein the patient receives a dose of between about 50-100, 100-200, 200-500, 500-1000, 1000-2000, 2000-5000, or 5000-10000 uCi.

145. The method of any of claims 126-144, which further comprises administering a kidney protectant to the patient.

146. The method of claim 145, wherein the kidney protectant comprises one or more of Clinisol, lysine, lysine/arginine, Gelofusine, or amifostine.

147. The method of any of claims 126-146, which results in a reduction in tumor growth fold change, measured from a baseline.

148. The method of any of claims 126-147, which results in a higher probability of survival at a given time point, compared to an expected course of the disorder without treatment.

149. Use of a compound of claim 21 or 22 for treating a disorder characterized by one or more GCC-expressing cells.

150. Use of the compound of claim 21 or 22 in the preparation of a medicament for treating a disorder characterized by one or more GCC-expressing cells.

151. A method of radiolabeling a compound of any one of claims 1-17, comprising:
(a) providing an amount of Gallium-68,
(b) purifying the amount of Gallium-68, thereby producing purified Gallium-68, and
(c) contacting about 45-65 μg of the compound of any one of claims 1-17 with the purified Gallium-68, in a buffer, for an incubation time of about 3-20 minutes, at a temperature of about 60-100° C. and a pH of about 3.0-4.5;
thereby producing a radiolabeled compound with a specific activity of about 25-33 MBq/nmol.

152. The method of claim 151, which comprises generating an amount of 68Ga via a 68Ge/68Ga generator.

153. The method of claim 151 or 152, wherein the Gallium-68 is provided as Gallium-68 chloride.

154. The method of any of claims 151-153, wherein purifying the amount of Gallium-68 comprises one or more of eluting the Gallium-68 from the generator with HCl thereby producing an eluate, loading the eluate into a cation column, and eluting the Gallium-68 from the cation column using acetone and HCl.

156. The method of claim 154, wherein the HCl used in eluting Gallium-68 from the generator is about 0.1M HCl.

157. The method of claim 154, wherein the Gallium-68 is eluted from the cation column using 98% acetone and 0.02M HCl.

158. The method of any of claims 151-157, wherein step (c) involves about 20-70 ug of the compound of any one of claims 1-17.

159. The method of claim 158, wherein step (c) involves about 55 ug of the compound of any one of claims 1-6 and 8-14.

160. The method of any of claims 151-159, wherein the buffer comprises citrate, acetate, or phosphate.

161. The method of any of claims 151-160, wherein the buffer comprises sodium acetate.

162. The method of any of claims 151-161, wherein the temperature is about 100 degrees.

163. The method of any of claims 151-162, wherein the pH is about 3.75-4.

164. The method of any of claims 151-163, wherein the compound a compound according to claim 17.

165. The method of any of claims 151-164, wherein the incubation time is about 6-10 minutes.

166. The method of any of claims 151-165, further comprising purifying the radiolabeled compound by buffer exchange.

167. The method of any of claims 151-166, further comprising sterile-filtering the radiolabeled compound using a 0.2 μm filter.

168. A method of determining a suitable injection volume of a Gallium-68-labeled compound of any of claims 1-17 to administer to a patient at a given time of injection, comprising computing the following formula:
[injection dose volume]=[radioactive dose at the time of injection, in mCi]/{([Radioactive count at the time of calibration, in mCi]/[Volume of composition at the time of injection, in ml]×EXP(−6.14E-1 hours−1)*[time between calibration and injection, in hours]}.
